SEQUENCE LISTING (16 sequences)

SEQ ID NO: 1 - DNA, 20 bp, Artificial Sequence
  Synthetic PCR Primers
gcagagctcgtttagtgaac

SEQ ID NO: 2 - DNA, 21 bp, Artificial Sequence
  Synthetic PCR Primers
gaaaatgggtagagatatgct

SEQ ID NO: 3 - DNA, 21 bp, Artificial Sequence
  Synthetic PCR Primers
atgagcttgtccttcctcctc

SEQ ID NO: 4 - DNA, 20 bp, Artificial Sequence
  Synthetic PCR Primers
tcgtttctcagcagctgttg

SEQ ID NO: 5 - DNA, 20 bp, Artificial Sequence
  Synthetic PCR Primers
catctgaactcaaagcgtgg

SEQ ID NO: 6 - protein, 25 aa, Artificial Sequence
  Synthetic construct of Beta 9 - Beta 10 loop in FGF-1
ENHYNTYISKKHAEKHWFVGLKKNG

SEQ ID NO: 7 - protein, 23 aa, Artificial Sequence
  Synthetic construct of Beta 9 - Beta 10 loop in FGF-2
SNNYNTYRSRKYTSWYVALKRTG

SEQ ID NO: 8 - protein, 22 aa, Artificial Sequence
  Synthetic construct of Beta 9 - Beta 10 loop in IL-1 Beta
NNKLEFESAQFPNWYISTSQAE

SEQ ID NO: 9 - DNA, 74 bp, Artificial Sequence
  Synthetic PCR Primers
cgaacgattggaatctaataactacaatacgtaccggtctgcgcagtttcctaactggtatgtggcacttaagc

SEQ ID NO: 10 - DNA, 76 bp, Artificial Sequence
  Synthetic PCR Primers
gtacgcttaagtgccacataccagttaggaaactgcgcagaccggtacgtattgtagttattagattccaatcgtt

SEQ ID NO: 11 - DNA, 32 bp, Artificial Sequence
  Synthetic PCR Primers
cgggatccgcccatggcggggcccgggacggc

SEQ ID NO: 12 - DNA, 28 bp, Artificial Sequence
  Synthetic PCR Primers
cggaattctgtgaaggtggtgatttccc

SEQ ID NO: 13 - DNA, 29 bp, Artificial Sequence
  Synthetic PCR Primers
cggaattcatggctgaaggggaaatcacc

SEQ ID NO: 14 - DNA, 65 bp, Artificial Sequence
  Synthetic PCR Primers
gctctagattaggcgtagtctgggacgtcgtatgggtagctcttagcagacattggaagaaaaag

SEQ ID NO: 15 - protein, 5 aa, Artificial Sequence
  Synthetic Construct
RKYTS

SEQ ID NO: 16 - protein, 5 aa, Artificial Sequence
  Synthetic Construct
AQFPN

(19) United States
(12) Patent Application Publication
(10) Pub. No.: US 2006/0286072 A1
(43) Pub. Date: Dec. 21, 2006

(54) Techniques and compositions for treating cardiovascular disease by in vivo gene delivery

(76) Inventors: Frank J. Giordano, Madison, CT (US); Wolfgang Dillmann, Solana Beach, CA (US); H. Kirk Hammond, La Jolla, CA (US)

Correspondence Address: MORRISON & FOERSTER LLP, 755 PAGE MILL RD, PALO ALTO, CA, 94304-1018, US

(21) Appl. No.: 11/236,221

(22) Filed: Sep. 26, 2005

Related U.S. Application Data
(63) Continuation of application No. 09/847,936, filed on May 3, 2001, now abandoned.
Application No. 09/847,936 is a continuation-in-part of application No. 09/609,080, filed on Jun. 30, 2000, now abandoned.
Application No. 09/609,080 is a continuation-in-part of application No. 09/435,156, filed on Nov. 5, 1999, now abandoned.
Application No. 09/435,156 is a continuation-in-part of application No. 08/722,271, filed on Dec. 29, 1997, now Pat. No. 6,100,242.
Application No. 08/722,271 is a continuation-in-part of application No. 08/485,472, filed on Jun. 7, 1995, now Pat. No. 5,792,453.
Application No. 08/485,472 is a continuation-in-part of application No. 08/396,207, filed on Feb. 28, 1995, now abandoned.
Application No. 09/847,936 is a continuation-in-part of application No. PCT/US00/30345, filed on Nov. 3, 2000.
Application No. 09/847,936 is a continuation-in-part of application No. PCT/US99/02702, filed on Feb. 9, 1999.
Application No. PCT/US99/02702 is a continuation-in-part of application No. 09/021,773, filed on Feb. 11, 1998, now abandoned.
Application No. 09/021,773 is a continuation-in-part of application No. 08/485,472, filed on Jun. 7, 1995, now Pat. No. 5,792,453.
Application No. 09/847,936 is a continuation-in-part of application No. 09/068,102, filed on Apr. 30, 1998, now abandoned.
Application No. 09/068,102 is a continuation of application No. 08/852,779, filed on May 7, 1997, now abandoned.

(51) Int. Cl. A61K 48/00; C12N 15/861

(52) U.S. Cl. 424/93.2; 514/44; 435/456

(57) ABSTRACT

Methods are provided for treating patients with cardiovascular disease, including heart disease and peripheral vascular disease. The preferred methods of the present invention involve in vivo delivery of genes, encoding angiogenic proteins or peptides, to the myocardium or to peripheral ischemic tissue, by introduction of a vector containing the gene into a blood vessel supplying the heart or into a peripheral ischemic tissue.

### CROSS REFERENCE TO RELATED CASES

This application is a continuation-in-part of U.S. application Ser. No. 09/609,080, filed Jun. 30, 2000, which is a continuation-in-part of U.S. application Ser. No. 09/435,156, filed Nov. 5, 1999, which is a continuation-in-part of U.S. application Ser. No. 08/722,271, filed Feb. 27, 1996 (now proceeding to issuance), which is a continuation-in-part of U.S. application Ser. No. 08/485,472, filed Jun. 7, 1995 (now issued as U.S. Pat. No. 5,792,453), which was a continuation-in-part of U.S. application Ser. No. 08/396,207, filed Feb. 28, 1995; and this application is a continuation-in-part of international application PCT/US00/30345, filed Nov. 3, 2000; and this application is a continuation-in-part of international application PCT/US99/02702 filed Feb. 9, 1999, which is a continuation-in-part of U.S. application Ser. No. 09/021,773, filed Feb. 11, 1998, which is a continuation-in-part of U.S. application Ser. No. 08/485,472, filed Jun. 7, 1995 (now issued as U.S. Pat. No. 5,792,453); and this application is a continuation-in-part of U.S. application Ser. No. 09/068,102, filed Apr. 30, 1998, which is a continuation of U.S. application Ser. No. 08/852,779, filed May 6, 1997 and is a continuation-in-part of U.S. application Ser. No. 09/132,167, filed Aug. 10, 1998. All of the above patent applications are incorporated by reference herein.

### STATEMENT REGARDING GOVERNMENT-SPONSORED RESEARCH

Certain of the work described herein was supported in part by grants from the United States Government under Grant Nos. VA-HL0281201, HL1768218 and IP50HL53773.01 awarded by the National Institutes of Health. The U.S. Government may have certain rights in this invention.

### FIELD OF THE INVENTION

The present invention relates to methods and compositions for treating cardiovascular disease, by in vivo gene therapy. More specifically, the present invention relates to techniques and polynucleotide constructs for treating heart disease and/or for treating peripheral vascular disease by in vivo delivery of angiogenic transgenes.

### BACKGROUND OF THE INVENTION

It has been reported by the American Heart Association (1995 Statistical Supplement), that about 60 million adults in the United States suffer from cardiovascular disease. Cardiovascular diseases are responsible for almost a million deaths annually in the United States representing over 40% of all deaths. Each year, in the United States, there are about 350,000 new cases of angina pectoris, a common condition of coronary artery disease characterized by transient periods of myocardial ischemia resulting in chest pain. Similarly, each year, some 400,000 patients are diagnosed with congestive heart failure “CHF”, another manifestation of heart disease that represents the most frequent non-elective cause of hospitalization in the U.S. In 1996, an estimated 725,000 people suffered from peripheral vascular disease, of whom over 100,000 would require a limb amputation.

Myocardial ischemia is an aspect of heart dysfunction that occurs when the heart muscle (the myocardium) does not receive adequate blood supply and is thus deprived of necessary levels of oxygen and nutrients. Myocardial ischemia may result in a variety of heart diseases including, for example, angina, heart attack and/or congestive heart failure. The most common cause of myocardial ischemia is atherosclerosis (also referred to as coronary artery disease or “CAD”), which causes blockages in the coronary arteries, blood vessels that provide blood flow to the heart muscle. Present treatments for myocardial ischemia include pharmacological therapies, coronary artery bypass surgery and percutaneous revascularization using techniques such as balloon angioplasty. Standard pharmacological therapy is predicated on strategies that involve either increasing blood supply to the heart muscle or decreasing the demand of the heart muscle for oxygen and nutrients. For example, increased blood supply to the myocardium can be achieved by agents such as calcium channel blockers or nitroglycerin. These agents are thought to increase the diameter of diseased arteries by causing relaxation of the smooth muscle in the arterial walls. Decreased demand of the heart muscle for oxygen and nutrients can be accomplished either by agents that decrease the hemodynamic load on the heart, such as arterial vasodilators, or those that decrease the contractile response of the heart to a given hemodynamic load, such as beta-adrenergic receptor antagonists. Surgical treatment of ischemic heart disease is generally based on the bypass of diseased arterial segments with strategically placed bypass grafts (usually saphenous vein or internal mammary artery grafts). Percutaneous revascularization is generally based on the use of catheters to reduce the narrowing in diseased coronary arteries. All of these strategies are used to decrease the number of, or to eradicate, ischemic episodes, but all have various limitations, some of which are discussed below.

Many patients with heart disease, including many of those whose severe myocardial ischemia resulted in a heart attack, are diagnosed as having congestive heart failure. Congestive heart failure is defined as abnormal heart function resulting in inadequate cardiac output to meet metabolic needs (Braunwald, E. (ed), In: Heart Disease, W. B. Saunders, Philadelphia, page 426, 1988). An estimated 5 million people in the United States suffer from congestive heart failure. Once symptoms of CHF are moderately severe, the prognosis is worse than most cancers in that only half of such patients are expected to survive for more than 2 years (Braunwald, E. (ed), In: Heart Disease, W. B. Saunders, Philadelphia, page 471-485, 1988). Medical therapy can initially attenuate the symptoms of CHF (e.g., edema, exercise intolerance and breathlessness), and in some cases prolong life. However, the prognosis for this disease, even with medical treatment, remains grim, and the incidence of CHF has been increasing (see, e.g., Baughman, K., Cardiology Clinics 13: 27-34, 1995). Symptoms of CHF include breathlessness, fatigue, weakness, leg swelling and exercise intolerance. On physical examination, patients with heart failure tend to have elevations in heart and respiratory rates, rales (an indication of fluid in the lungs), edema, jugular venous distension, and, in general, enlarged hearts. The most common cause of CHF is atherosclerosis which, as discussed above, causes blockages in the coronary arteries that supply blood to the heart muscle. Thus, congestive heart failure is most commonly associated with coronary artery disease that is so severe in scope or abruptness that it results in the development of chronic or acute heart failure. In such patients, extensive and/or abrupt occlusion of one or more coronary arteries precludes adequate blood flow to the myocardium, resulting in severe ischemia and, in some cases, myocardial infarction or death of heart muscle. The consequent myocardial necrosis tends to be followed by progressive chronic heart failure or an acute low output state—both of which are associated with high mortality.

Most patients with congestive heart failure tend to develop enlarged, poorly contracting hearts, a condition referred to as “dilated cardiomyopathy” (or DCM, as used herein). DCM is a condition of the heart typically diagnosed by the finding of a dilated, hypocontractile left and/or right ventricle. Again, in the majority of cases, the congestive heart failure associated with a dilated heart is the result of coronary artery disease, often so severe that it has caused one or more myocardial infarcts. In a significant minority of cases, however, DCM can occur in the absence of characteristics of coronary artery disease (e.g., atherosclerosis). In a number of cases in which the dilated cardiomyopathy is not associated with CAD, the cause of DCM is known or suspected. Examples include familial cardiomyopathy (such as that associated with progressive muscular dystrophy, myotonic muscular dystrophy, Freidrich's ataxia, and hereditary dilated cardiomyopathy), infections resulting in myocardial inflammation (such as infections by various viruses, bacteria and other parasites), noninfectious inflammations (such as those due to autoimmune diseases, peripartum cardiomyopathy, hypersensitivity reactions or transplantation rejections), metabolic disturbances causing myocarditis (including nutritional, endocrinologic and electrolyte abnormalities) and exposure to toxic agents causing myocarditis (including alcohol, as well as certain chemotherapeutic drugs and catecholamines). In the majority of non-CAD DCM cases, however, the cause of disease remains unknown and the condition is thus referred to as “idiopathic dilated cardiomyopathy” (or “IDCM”). Despite the potential differences in underlying causation, most patients with severe CHF have enlarged, thin-walled hearts (i.e., DCM) and most of those patients exhibit myocardial ischemia (even though some of them may not have apparent atherosclerosis). Furthermore, patients with DCM can experience angina pectoris even though they may not have severe coronary artery disease.

The occurrence of CHF poses several major therapeutic concerns, including progressive myocardial injury, hemodynamic inefficiencies associated with the dilated heart, the threat of systemic emboli, and the risk of ventricular arrhythmias. Traditional revascularization is not an option for treatment of non-CAD DCM, because occlusive coronary disease is not the primary problem. Even for those patients for which the cause of DCM is known or suspected, the damage is typically not readily reversible. For example, in the case of adriamycin-induced cardiotoxicity, the cardiomyopathy is generally irreversible and results in death in over 60% of afflicted patients. For some patients with DCM, the cause itself is unknown. As a result, there are no generally applied treatments for DCM. Physicians have traditionally focused on alleviating the symptoms presented in a patient exhibiting DCM (e.g., by relieving fluid retention with diuretics, and/or reducing the demand of the heart muscle for oxygen and nutrients with angiotensin converting enzyme inhibitors). As a result, approximately 50% of the patients exhibiting DCM die within two years of diagnosis, often from sudden cardiac arrest associated with ventricular arrhythmias. “Ventricular remodeling” is an aspect of heart disease that often occurs after myocardial infarction and often results in further decrease in ventricular function. In many cases, after a myocardial infarct heals, continued ischemia in the border region between the healed infarct and normal tissue and other factors lead to a dilation and/or remodeling of the remaining heart tissue. This dilating or remodeling, while initially adaptive, often leads further impairment of ventricular function. Dilation of the whole heart occurs in about 50% of patients who have such infarcts, and remodeling usually develops within a few months after a myocardial infarction although it can occur as early as 1-2 weeks after the infarct. Poor left ventricular function is the best single predictor of adverse outcome following myocardial infarction. Thus, preventing ventricular remodeling after myocardial infarction would be beneficial. One approach to try to prevent ventricular remodeling is to treat patients who have suffered a myocardial infarction with angiotensin converting enzyme (“ACE”inhibitors (see, e.g., McDonald, K. M., Trans. Assoc. Am. Physicians 103:229-235, 1990; Cohn, J. Clin. Cardiol. 18 (Suppl. IV) IV-4-IV-12, 1995). However, these agents are only somewhat effective at preventing deleterious ventricular remodeling and new therapies are needed.

Present treatments for CHF include pharmacological therapies, coronary revascularization procedures and heart transplantation. Pharmacological therapies for CHF have been directed toward increasing the force of contraction of the heart (by using inotropic agents such as digitalis and beta-adrenergic receptor agonists), reducing fluid accumulation in the lungs and elsewhere (by using diuretics), and reducing the work of the heart (by using agents that decrease systemic vascular resistance such as angiotensin converting enzyme inhibitors). Beta-adrenergic receptor antagonists have also been tested. While such pharmacological agents can improve symptoms, and potentially prolong life, the prognosis in most cases remains dismal.

Some patients with heart failure due to associated coronary artery disease can benefit, at least temporarily, by revascularization procedures such as coronary artery bypass surgery and angioplasty. Such procedures are of potential benefit when the heart muscle is not dead but may be dysfunctional because of inadequate blood flow. If normal coronary blood flow is restored, previously dysfunctional myocardium may contract more normally, and heart function may improve. However, if the patient has an inadequate microvascular bed (e.g., as may be found in more severe CHF patients), revascularization will rarely restore cardiac function to normal or near-normal levels, even though mild improvements are sometimes noted. In addition, the incidence of failed bypass grafts and restenosis following angioplasty poses further risks to patients treated by such methods. Heart transplantation can be a suitable option for CHF patients who have no other confounding diseases and are relatively young, but this is an option for only a small number of such patients, and only at great expense. In sum, it can be seen that CHF has a very poor prognosis and responds poorly to current therapies.

Further complicating the physiological conditions associated with CHF are various natural adaptations that tend to occur in patients with dysfunctional hearts. Although these natural responses can initially improve heart function, they often result in other problems that can exacerbate the disease, confound treatment, and have adverse effects on survival. There are three such adaptive responses commonly observed in CHF patients: (i) volume retention induced by changes in sodium reabsorption, which expands plasma volume and initially improves cardiac output; (ii) cardiac enlargement (from dilation and hypertrophy) which can increase stroke volume while maintaining a relatively normal wall tension; and (iii) increased norepinephrine release from adrenergic nerve terminals impinging on the heart which, by interacting with cardiac beta-adrenergic receptors, tends to increase heart rate and force of contraction, thereby increasing cardiac output. However, each of these three natural adaptations tends ultimately to fail for various reasons. In particular, fluid retention tends to result in edema and retained fluid in the lungs that impairs breathing. Heart enlargement can lead to deleterious left ventricular remodeling with subsequent severe dilation and increased wall tension, thus exacerbating CHF. Finally, long-term exposure of the heart to norepinephrine tends to make the heart unresponsive to adrenergic stimulation and is linked with poor prognosis.

Diseases of the peripheral vasculature, like heart disease, often result from restricted blood flow to the tissue (e.g. skeletal muscle) which (like cardiac disease) becomes ischemic, particularly when metabolic needs increase (such as with exercise). Thus, atherosclerosis present in a peripheral vessel may cause ischemia in the tissue supplied by the affected vessel. This problem, known as peripheral arterial occlusive disease (PAOD), most frequently affects in the lower limbs of patients. As with other forms of cardiovascular disease, this condition or at least some of its symptoms, may be treated by using drugs, such as aspirin or other agents that reduce blood viscosity, or by surgical intervention, such as arterial grafting, surgical removal of fatty plaque deposits or by endovascular treatments, such as angioplasty. While symptoms may be improved, the effectiveness of such treatments is typically inadequate, for reasons similar to those referred to above.

Recently, investigations into treatments for cardiovascular disease have turned to therapeutics related to angiogenesis. Angiogenesis refers generally to the development and differentiation of blood vessels. A number of proteins, typically referred to as “angiogenic proteins,” are known to promote angiogenesis. Such angiogenic proteins include members of the fibroblast growth factor (FGF) family, the vascular endothelial growth factor (VEGF) family, the platelet-derived growth factor (PDGF) family, the insulin-like growth factor (IGF) family, and others (as described in more detail below and in the art). For example, the FGF and VEGF family members have been recognized as regulators of angiogenesis during growth and development. Their role in promoting angiogenesis in adult animals has recently been examined (as discussed below). The angiogenic activity of the FGF and VEGF families has bee examined. For example, it has been shown that acidic FGF (“aFGF”) protein, within a collagen-coated matrix, when placed in the peritoneal cavity of adult rats, resulted in a well vascularized and normally perfused structure (Thompson et al., Proc. Natl. Acad. Sci. USA, 86: 7928-7932, 1989). Injection of basic FGF (“bFGF”) protein into adult canine coronary arteries during coronary occlusion reportedly led to decreased myocardial dysfunction, smaller myocardial infarctions, and increased vascularity in the bed at risk (Yanagisawa-Miwa et al., Science, 257: 1401-1403, 1992). Similar results have been reported in animal models of myocardial ischemia using bFGF protein (Harada et al., J. Clin. Invest., 94: 623-630, 1994; Unger et al., Am. J. Physiol., 266: H1588-H-1595, 1994). An increase in collateral blood flow was shown in dogs treated with VEGF protein (Banai et al. Circulation 89: 2183-2189, 1994).

However, difficulties associated with the potential use of such protein infusions to promote cardiac angiogenesis include: achieving proper localization for a sufficient period of time, and ensuring that the protein is and remains in the proper form and concentration needed for uptake and the promotion of an angiogenic effect within cells of the myocardium. A protein concentration which is high initially (e.g., following bolus infusion) but then drops rapidly (with clearance by the body) can be both toxic and ineffective. Another difficulty is the need for repeated infusion or injection of the protein.

Some publications postulated on the use of gene transfer for the treatment or prevention of disease, including certain heart diseases. See, for example, French, “Gene Transfer and Cardiovascular Disorders,” Herz 18:222-229, 1993; Williams, “Prospects for Gene Therapy of Ischemic Heart Disease,” American Journal of Medical Sciences 306:129-136, 1993; Schneider and French, “The Advent of Adenovirus: Gene Therapy for Cardiovascular Disease,” Circulation 88:1937-1942, 1993; and Mazur et al., “Coronary Restenosis and Gene Therapy,” Molecular and Cellular Pharmacology, 21:104-111, 1994. Additionally, some groups have suggested in vivo gene transfer into the myocardium using plasmids, retrovirus, adenovirus and other vectors (see e.g., Barr et al., Supplement II, Circulation, 84(4): Abstract 1673, 1991; Barr et al., Gene Ther., 1: 51-58, 1994; French et al., Circulation, 90(5): 2402-2413, 1994; French et al., Circulation, 90(5): 2414-2424, 1994; French et al., Circulation, 90: 1517 Abstract No. 2785, 1994; Leiden, et al., WO94/11506 (26 May 1994); Guzman et al., Circ. Res., 73(6): 1202-1207, 1993; Kass-Eisler et al., Proc. Natl. Acad. Sci. USA, 90: 11498-11502, 1993; Mühlahauser et al., Hum. Gene Ther., 6: 1457-1465, 1995; Mühlahauser et al. Circ. Res., 77(6): 1077-1086, 1995; and Rowland et al., Am. Thorac. Surg., 60(3): 721-728, 1995.

In general, however, these reports provided little more than suggestions or wishes for potential therapies. Of those providing animal data, most did not employ disease models suitably related to actual in vivo conditions. Moreover, the attempted in vivo methods generally suffered from one or more of the following deficiencies: inadequate transduction efficiency and transgene expression; marked immune response to the vectors used, including inflammation and tissue necrosis; and importantly, a relative inability to target transduction and transgene expression to the organ of interest (e.g., gene transfer targeted to the heart resulted in the transgene also being delivered to non-cardiac sites such as liver, kidneys, lungs, brain and testes of the test animals). By way of example, the insertion of a transgene into a rapidly dividing cell population will result in substantially reduced duration of transgene expression. Examples of such cells include endothelial cells, which make up the inner layer of all blood vessels, and fibroblasts, which are dispersed throughout the heart. Targeting the transgene so that only the desired cells will receive and express the transgene, and so that the transgene will not be systemically distributed, are also critically important considerations. If this is not accomplished, systemic expression of the transgene and problems attendant thereto will result. For example, inflammatory infiltrates have been documented after adenovirus-mediated gene transfer in liver (Yang et al. Proc. Natl. Acad. Sci. U.S.A., 91: 4407, 1994). Additionally, inflammatory infiltrates were documented in the heart after direct intramyocardial injection through a needle inserted into the myocardial wall (French et al., Circulation, 90(5): 2414-2424, 1994).

A method for treating certain forms of congestive heart failure associated with beta-adrenergic signaling has recently been demonstrated by Hammond et al. in PCT publication WO 98/10085, published 12 Mar. 1998. That method involves the delivery of genes encoding elements of the beta-adrenergic signaling pathway to the heart of a patient with heart disease associated with a reduction in beta-adrenergic signaling.

### SUMMARY OF THE INVENTION

The present invention relates to methods and compositions for treating cardiovascular disease comprising delivering a transgene encoding an angiogenic protein or peptide to affected tissue by introducing a vector comprising the transgene into said tissue wherein the transgene is expressed and disease symptoms ameliorated. For example, contractile function and/or blood flow in the heart can be increased by introduction of a transgene-containing vector into at least one coronary artery of a patient, wherein the transgene is delivered to the myocardium and therein expressed. Methods are also provided for use in peripheral vascular diseases such as peripheral arterial occlusive disease (PAOD). As described and illustrated herein, these methods are thus useful for treating heart disease, peripheral vascular disease and similar disorders.

The present invention provides a method for increasing blood flow in an ischemic tissue of a patient, comprising delivering an angiogenic protein or peptide to an ischemic region of said tissue by introducing a vector comprising the transgene to the tissue, whereby the transgene is expressed in the tissue, and blood flow in the tissue is increased. In one aspect, the vector, comprising a transgene encoding an angiogenic protein or peptide, is introduced into ischemic skeletal muscle, wherein the angiogenic protein or peptide is expressed and causes an increase in blood flow and a decrease in ischemia in the tissue. In an alternative embodiment, the vector is introduced into a blood vessel supplying blood to the ischemic tissue (e.g. by introduction into a coronary artery supplying the myocardium or into a peripheral artery, such as a femoral artery, supplying skeletal muscle). The vectors employed in the invention can be a plasmid or preferably a viral vector, for example a replication-deficient adenovirus. Various aspects and therapeutic applications of the present invention are described and illustrated below.

In one aspect, the present invention provides a method for increasing contractile function in the heart of a patient, comprising delivering a transgene encoding an angiogenic protein or peptide to the myocardium of the patient by introducing a vector comprising the transgene to the myocardium (preferably by delivery to one or more coronary arteries), wherein the transgene is delivered to the myocardium and expressed, and contractile function in the heart is increased. The transgene may be introduced by, for example, intracoronary injection into one or more coronary arteries or saphenous vein or internal mammary artery grafts supplying blood to the myocardium. The transgene preferably encodes at least one angiogenic protein or peptide. The vectors employed in the invention can be a plasmid or preferably a viral vector, including, by way of illustration, a replication-deficient adenovirus. By injecting the viral vector stock (preferably containing relatively few or no wild-type virus), deeply (at least about 1 cm) into the lumen of one or both coronary arteries or grafts (preferably into both right and left coronary arteries or grafts), and preferably in an amount of 10⁷-10¹³ viral particles as determined by optical densitometry (more preferably 10⁹-10¹¹ viral particles), it is possible to locally transfect a desired number of cells, especially cardiac myocytes, in the affected myocardium with angiogenic protein- or peptide-encoding genes, thereby maximizing therapeutic efficacy of gene transfer, and minimizing undesirable angiogenesis at extracardiac sites and the possibility of an inflammatory response to viral proteins. If a cardiomyocyte-specific promoter is used expression can be further limited to the cardiac myocytes so as to further reduce the potentially harmful effects of angiogenesis in non-cardiac tissues such as the retina.

Kits and compositions that can be used in accordance with the therapeutic techniques are also provided.

### BRIEF DESCRIPTION OF THE DRAWINGS

FIG. 1 graphically presents percent wall thickening during pacing in a porcine model of congestive heart failure. Percent wall thickening was assessed sequentially in the interventricular septum and lateral wall before pacing (day 0) and every 7 days as heart failure progressed (as described in Example 1). Symbols represent mean values; error bars denote one standard deviation (1 SD). Two-way ANOVA (repeated measures) showed that percent wall thickening was affected by duration of pacing (P<0.001) and by region (P=0.001). Furthermore, the pattern of change in wall thickening was different between the two regions (P<0.0001). Mean values for percent wall thickening at each time point were tested for differences between the two regions post hoc by the Tukey method; P values for these analyses are shown beneath the error bars.

FIGS. 2A and 2B graphically present subendocardial blood flow during pacing in a porcine model of congestive heart failure, as described in Example 1.

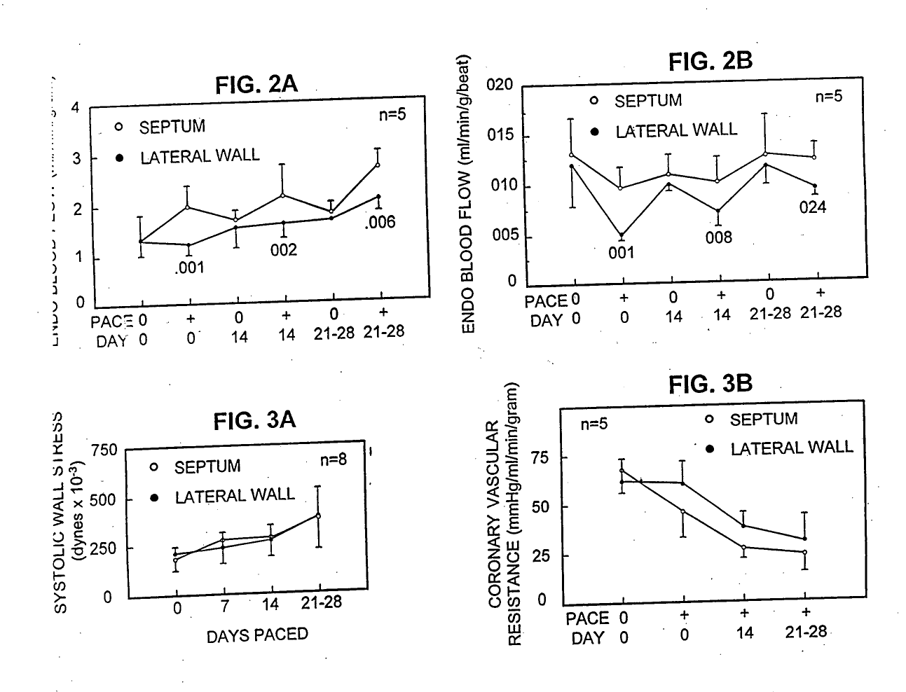

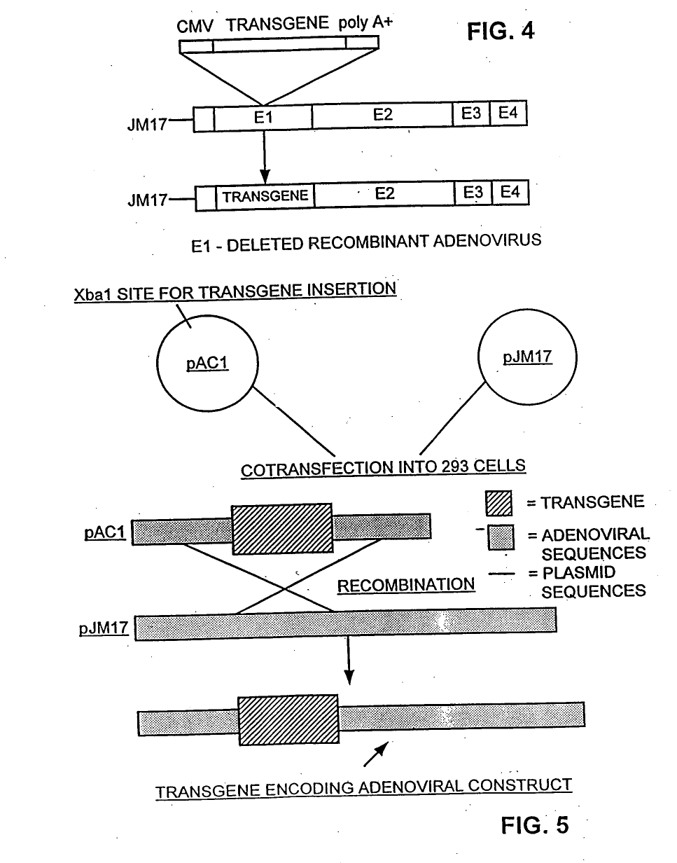

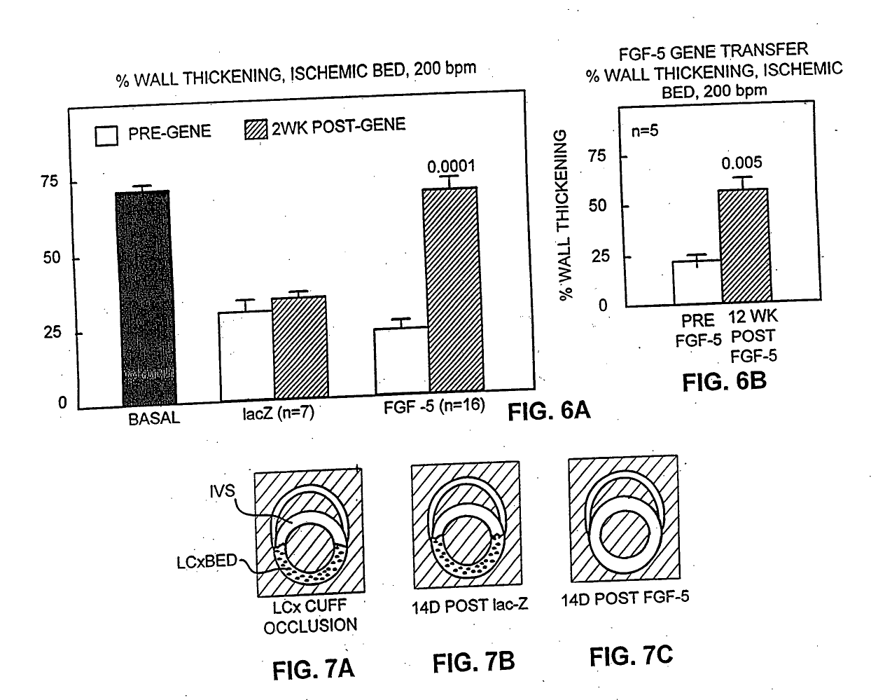

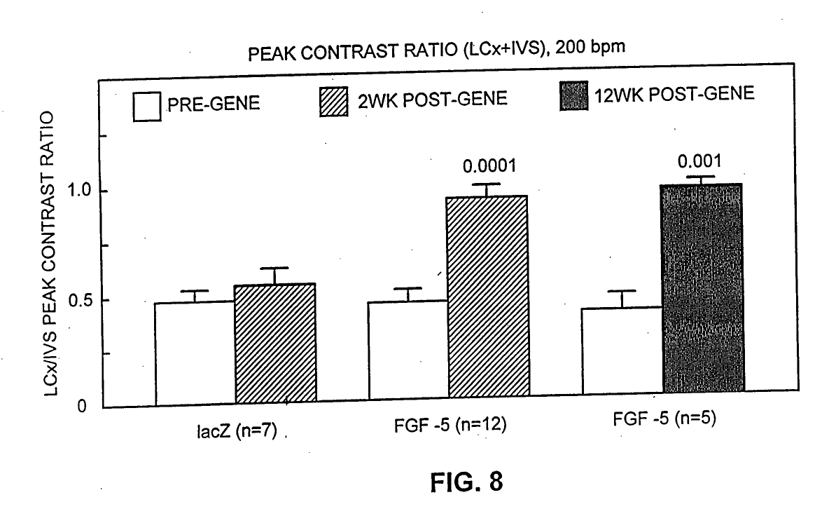

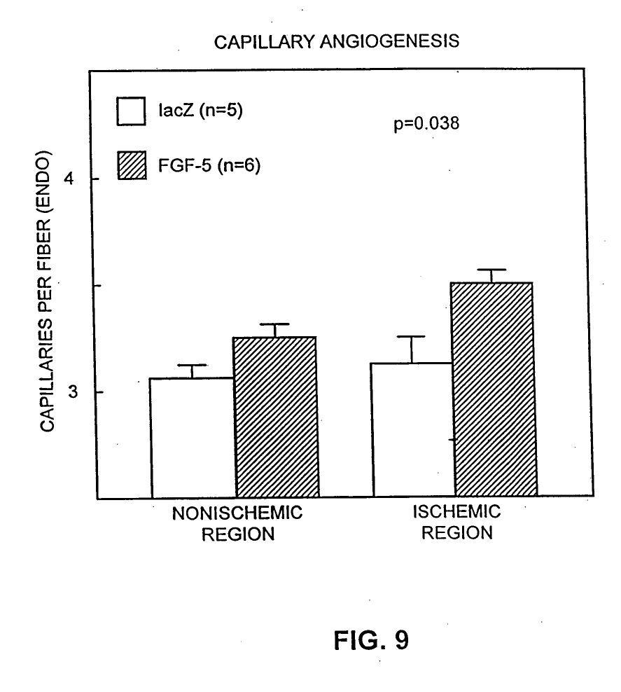

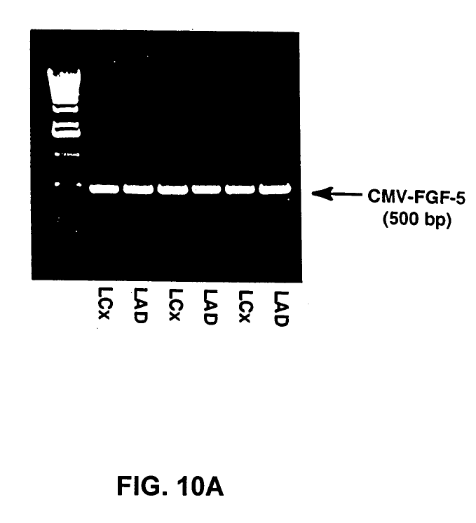

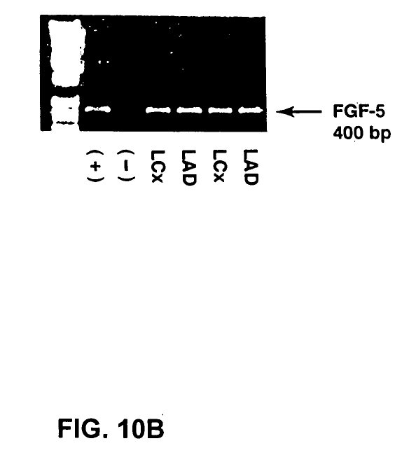

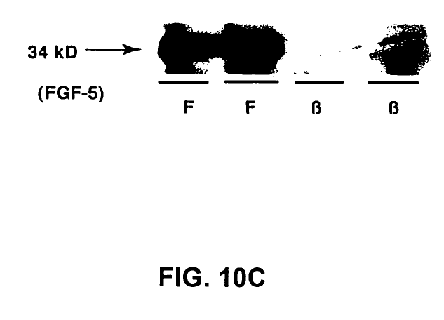

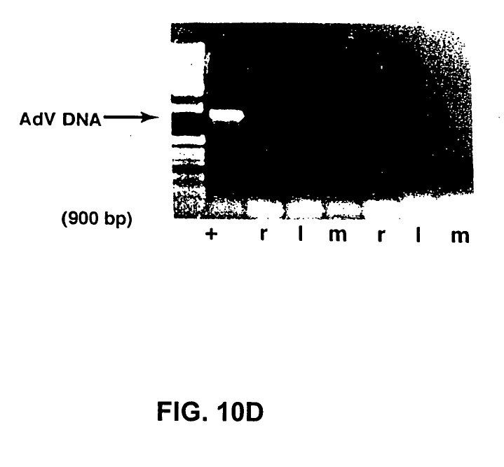

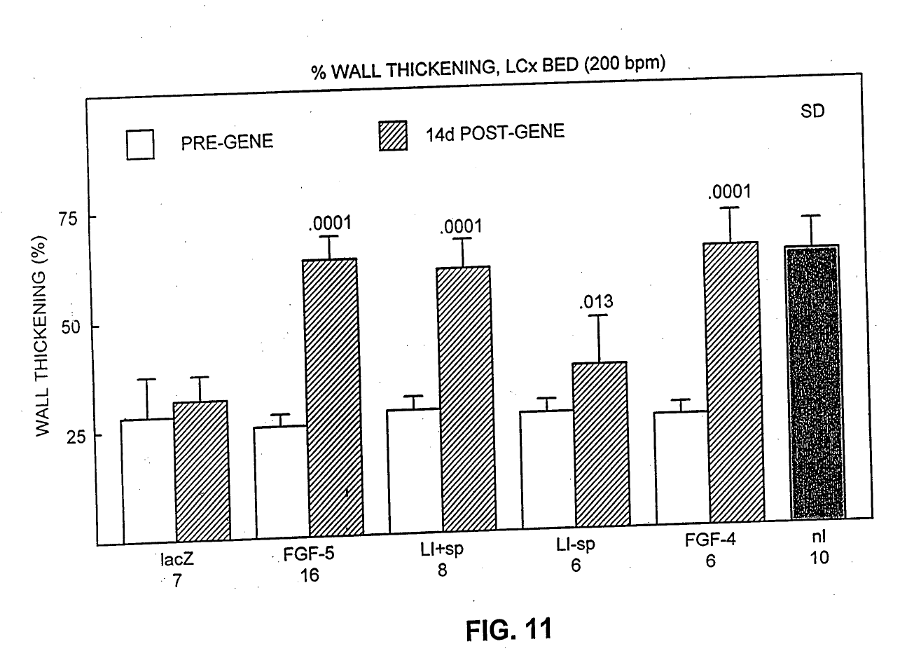

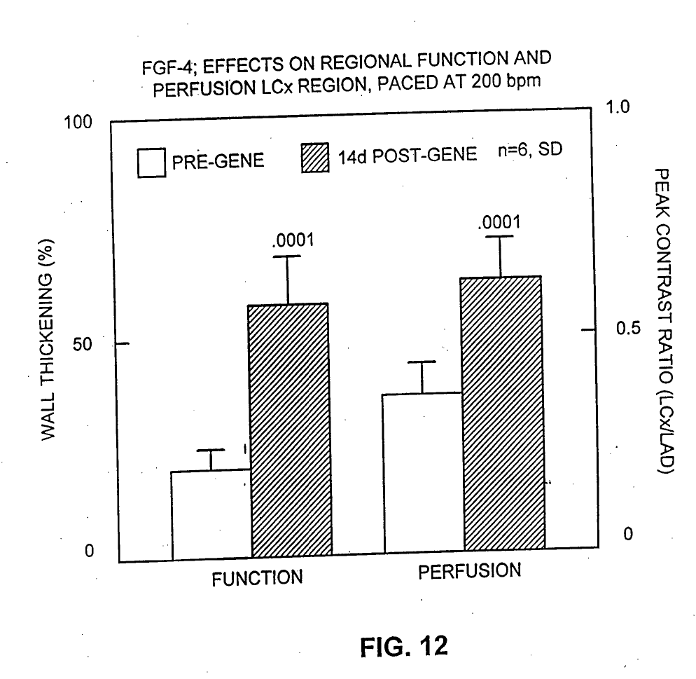

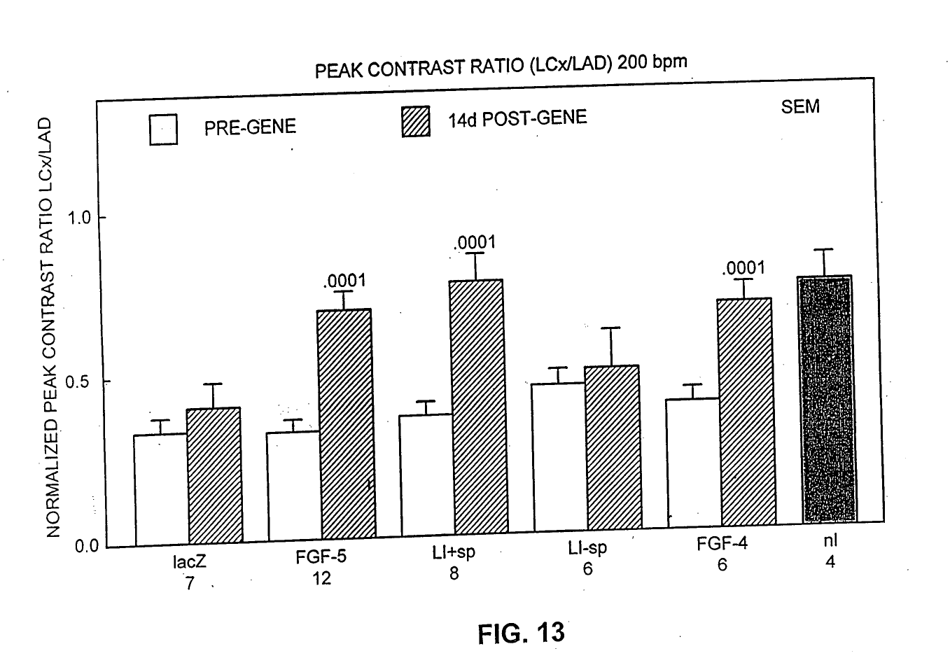

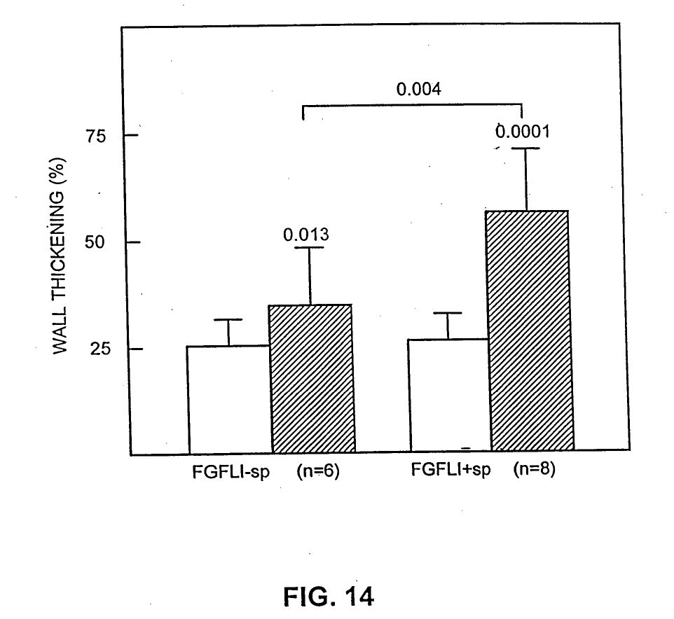

For FIG. 2A, subendocardial (endo) blood flow was assessed sequentially in the interventricular septum and lateral wall under the conditions listed along the x axis. Day refers to the day of sustained pacing that measurements were obtained (0, initiation of pacing; 14, 14 days; 21-28, 21 to 28 days). PACE refers to whether blood flow determinations were obtained with pacemaker activated (+) or inactivated (0). Pacemaker rate was 225 bpm. (See Table 3 herein for numerical values.) Symbols represent mean values; error bars denote 1 SD. Two-way ANOVA (repeated measures) showed that subendocardial blood flow was affected by duration of pacing (P=0.0001) and by region (P=0.017). Furthermore, the pattern of change in subendocardial blood flow was different between the two regions (P<0.006). Mean values for subendocardial blood flow at each time point were tested for differences between the two regions post hoc by Tukey analyses; P values for these analyses are shown beneath the error bars.

For FIG. 2B, subendocardial blood flow per beat was assessed sequentially in the interventricular septum and lateral wall under the conditions listed along the x axis. Symbols and conditions are as in FIG. 2A. (See Table 4 herein for numerical values.) Two-way ANOVA (repeated measures) showed that subendocardial blood flow per beat was affected by duration of pacing (P=0.0001) and by region (P=0.0198).

FIG. 3A graphically presents meridional end-systolic wall stress as assessed sequentially in the interventricular septum and lateral wall before pacing (day 0) and every 7 days as heart failure progressed (described in Example 1). Two-way ANOVA (repeated measures) showed that systolic wall stress was affected by duration of pacing (P<0.0001). However, the pattern of systolic wall stress was similar in both regions. Measurements were made with pacemakers inactivated.

FIG. 3B graphically presents coronary vascular resistance during pacing in a porcine model of congestive heart failure, as described in Example 1. An index of coronary vascular resistance was assessed sequentially in the interventricular septum and lateral wall under the conditions listed along the x axis. Symbols and conditions are the same as in FIG. 2. Two-way ANOVA (repeated measures) showed that the coronary vascular resistance index was affected by duration of pacing (P=0.0001) and by region (P=0.013). Furthermore, the pattern of change in coronary vascular resistance was different between the two regions (P=0.0012). Mean values for coronary vascular resistances at each time point were tested for differences between the two regions post hoc by Tukey analyses. This analysis showed that coronary vascular resistance was higher in the lateral wall than in the septum directly after the initiation of pacing (P value below error bar).

FIG. 4 shows a schematic of the construction of an exemplary replication-defective recombinant adenovirus vector useful for gene transfer, as described in the Examples below.

FIG. 5 is a schematic figure which shows rescue recombination construction of a transgene-encoding adenovirus.

FIGS. 6A and 6B graphically present the regional contractile function of the treated animals, as described in Example 5. FIG. 6A shows results of animals examined 2 weeks post gene transfer and FIG. 6B shows results 12 weeks post gene transfer.

FIGS. 7A, 7B and 7C show diagrams corresponding to myocardial contrast echocardiographs. White areas denote contrast enhancement (more blood flow) and dark areas denote decreased blood flow. FIG. 7A illustrates acute LCx occlusion in a normal pig. FIG. 7B illustrates the difference in contrast enhancement between IVS and LCx bed 14 days after gene transfer with lacZ, indicating different blood flows in two regions during atrial pacing (200 bpm). In FIG. 7C, contrast enhancement appears equivalent in IVS and LCx bed 14 days after gene transfer with FGF-5, indicating similar blood flows in the two regions during atrial pacing. These results are described in Example 5.

FIG. 8 shows the peak contrast ratio (a correlate of blood flow) expressed as the ratio of the peak video intensity in the ischemic region (LCx bed) divided by the peak video intensity in the interventricular septum (IVS), measured from the video images using a computer-based video analysis program during atrial pacing (200 bpm) before and 14±1 days after gene transfer with lacZ (control gene) and with FGF-5, and in 5 animals, 12 weeks after FGF-5 gene transfer (described in Example 5). Blood flow to the ischemic bed remained 50% of normal after gene transfer with the control gene but increased 2-fold above normal after gene transfer with FGF-5 (p=0.0018), an effect that persisted for at least 12 weeks.

FIG. 9 shows vessel number as quantitated by microscopic analysis in the ischemic and nonischemic regions after gene transfer with FGF-5 and with lacZ (described in Example 5). There was increased capillary number surrounding each fiber in the ischemic and nonischemic regions of animals that received FGF-5 gene transfer (p<0.038) compared to animals that received the lacZ gene.

FIGS. 10A, 10B and 10C are from gels documenting DNA, mRNA and protein expression after gene transfer of an angiogenic transgene to the myocardium according to the present invention (as described in Example 5). FIG. 10D is from a gel following PCR amplification demonstrating the absence of any detectable gene transfer to the retina, liver or skeletal muscle of treated animals (as described in Example 5).

FIG. 11 shows a comparison of wall thickening achieved with in vivo gene transfer using different angiogenic gene constructs, FGF-4, FGF-5 and FGF-2LI +/−sp (i.e. FGF-2LI plus or minus secretion signal peptide), as described in examples 6 and 7.

FIG. 12 shows that improved function in the ischemic region after FGF-4 gene transfer (as indicated by wall thickening) was associated with improved regional perfusion.

FIG. 13 shows a comparison of perfusion (blood flow) resulting from injection of FGF-4, FGF-5 or FGF-2LI +/−sp (=FGF-2LI plus or minus signal peptide), as described in Examples 6 and 7.

FIG. 14 shows a comparison of wall thickening as a result of gene transfer with FGF-2 plus (FGF-2LI+sp) or minus secretion signal peptide (FGF-2LI-sp), as described in Example 7.

### DETAILED DESCRIPTION OF PREFERRED EMBODIMENTS

Definitions

“Heart disease” refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure.

“Myocardial ischemia” is a condition in which the heart muscle does not receive adequate levels of oxygen and nutrients, which is typically due to inadequate blood supply to the myocardium (e.g., as a result of coronary artery disease).

“Heart failure” is clinically defined as a condition in which the heart does not provide adequate blood flow to the body to meet metabolic demands. Symptoms include breathlessness, fatigue, weakness, leg swelling, and exercise intolerance. On physical examination, patients with heart failure tend to have elevations in heart and respiratory rates, rales (an indication of fluid in the lungs), edema, jugular venous distension, and, in many cases, enlarged hearts. Patients with severe heart failure suffer a high mortality; typically 50% of the patients die within two years of developing the condition. In some cases, heart failure is associated with severe coronary artery disease (“CAD”), typically resulting in myocardial infarction and either progressive chronic heart failure or an acute low output state, as described herein and in the art. In other cases, heart failure is associated with dilated cardiomyopathy without associated severe coronary artery disease.

“Peripheral vascular disease” refers to acute or chronic dysfunction of the peripheral (i.e., non-cardiac) vasculature and/or the tissues supplied thereby. As with heart disease, peripheral vascular disease typically results from an inadequate blood flow to the tissues supplied by the vasculature, which lack of blood may result, for example, in ischemia or, in severe cases, in tissue cell death. Aspects of peripheral vascular disease include, without limitation, peripheral arterial occlusive disease (PAOD) and peripheral muscle ischemia. Frequently, symptoms of peripheral vascular disease are manifested in the extremities of the patient, especially the legs.

As used herein, the terms “having therapeutic effect” and “successful treatment” carry essentially the same meaning. In particular, a patient suffering from heart disease is successfully “treated” for the condition if the patient shows observable and/or measurable reduction in or absence of one or more of the symptoms of heart disease after receiving an angiogenic factor transgene according to the methods of the present invention. Reduction of these signs or symptoms may also be felt by the patient. Thus, indicators of successful treatment of heart disease conditions include the patient showing or feeling a reduction in any one of the symptoms of angina pectoris, fatigue, weakness, breathlessness, leg swelling, rales, heart or respiratory rates, edema or jugular venous distension. The patient may also show greater exercise tolerance, have a smaller heart with improved ventricular and cardiac function, and in general, require fewer hospital visits related to the heart condition. The improvement in cardiovascular function may be adequate to meet the metabolic needs of the patient and the patient may not exhibit symptoms under mild exertion or at rest. Many of these signs and symptoms are readily observable by eye and/or measurable by routine procedures familiar to a physician. Indicators of improved cardiovascular function include increased blood flow and/or contractile function in the treated tissues. As described below, blood flow in a patient can be measured by thallium imaging (as described by Braunwald in Heart Disease, 4^{th} ed., pp. 276-311 (Saunders, Philadelphia, 1992)) or by echocardiography (described in Examples 1 and 5 and in Sahn, D J., et al., Circulation. 58:1072-1083, 1978). Blood flow before and after angiogenic gene transfer can be compared using these methods. Improved heart function is associated with decreased signs and symptoms, as noted above. In addition to echocardiography, one can measure ejection fraction (LV) by nuclear (non-invasive) techniques as is known in the art. Blood flow and contractile function can likewise be measured in peripheral tissues treated according to the present invention.

An “angiogenic protein or peptide” refers to any protein or peptide capable of promoting angiogenesis or angiogenic activity, i.e. blood vessel development.

A “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides.

“Recombinant,” as applied to a polynucleotide, means that the polynucleotide is the product of various combinations of cloning, restriction and/or ligation steps, and other procedures that result in a construct that is distinct from a polynucleotide found in nature.

A “gene” or “transgene” refers to a polynucleotide or portion of a polynucleotide comprising a sequence that encodes a protein. For most situations, it is desirable for the gene to also comprise a promoter operably linked to the coding sequence in order to effectively promote transcription. Enhancers, repressors and other regulatory sequences may also be included in order to modulate activity of the gene, as is well known in the art. (See, e.g., the references cited below).

The terms “polypeptide,” “peptide,” and “protein” are used interchangeably to refer to polymers of amino acids of any length. These terms also include proteins that are post-translationally modified through reactions that include glycosylation, acetylation and phosphorylation.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art (and identified in databases such as GenBank) and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art (and identified in databases such as GenBank) and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition of two or more components, wherein the components so described are in a relationship permitting them to function in their intended manner. A promoter is operably linked to a gene or coding sequence if the promoter controls transcription of the gene or coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “replicon” refers to a polynucleotide comprising an origin of replication which allows for replication of the polynucleotide in an appropriate host cell. Examples include chromosomes of a target cell into which a heterologous nucleic acid might be integrated (e.g., nuclear and mitochondrial chromosomes), as well as extrachromosomal replicons (such as replicating plasmids and episomes).

“Gene delivery”, “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stable or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced into a cell of such organism in vivo.

A “vector” (sometimes referred to as a gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum. In preferred aspects of the present invention for treating heart disease, vectors comprising angiogenic transgenes are introduced directly into vascular conduits supplying blood to the myocardium. Such vascular conduits include the coronary arteries as well as vessels such as saphenous veins or internal mammary artery grafts.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

An “individual” or a “patient” refers to a mammal, preferably a large mammal, most preferably a human.

“Treatment” or “therapy” as used herein refers to administering, to an individual patient, agents that are capable of eliciting a prophylactic, curative or other beneficial effect on the individual.

“Gene therapy” as used herein refers to administering, to an individual patient, vectors comprising a therapeutic gene or genes.

A “therapeutic polynucleotide” or “therapeutic gene” refers to a nucleotide sequence that is capable, when transferred to an individual, of eliciting a prophylactic, curative or other beneficial effect in the individual.

### REFERENCES

The practice of the present invention will employ, unless otherwise indicated, conventional techniques of molecular biology and the like, which are within the skill of the art. Such techniques are explained in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al. eds., 1987 and updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology (R. Wu et al. eds., Academic Press 1989); PCR: A Practical Approach (M. McPherson et al., IRL Press at Oxford University Press 1991); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2nd Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Techniques in Immunocytochemistry, (G. Bullock & P. Petrusz eds., Academic Press 1982, 1983, 1985, 1989); Handbook of Experimental Immunology, (D. Weir & C. Blackwell, eds.); Cellular and Molecular Immunology (A. Abbas et al., W.B. Saunders Co. 1991, 1994); Current Protocols in Immunology (J. Coligan et al. eds. 1991); the series Annual Review of Immunology; the series Advances in Immunology; Oligonucleotide Synthesis (M. Gait ed., 1984); Animal Cell Culture (R. Freshney ed., IRL Press 1987); the series Arteriosclerosis, Thrombosis and Vascular Biology (Lippincott, Williams & Wilkins publishers for the American Heart Association); the series Circulation (Lippincott, Williams & Wilkins publishers for the American Heart Association); and the series Circulation Research (Lippincott, Williams & Wilkins publishers for the American Heart Association).

Additional references describing delivery and logistics of surgery which may be used in the methods of the present invention include the following: Topol, EJ (ed.), The Textbook of Interventional Cardiology, 2nd Ed. (W.B. Saunders Co. 1994); Rutherford, R B, Vascular Surgery, 3rd Ed. (W.B. Saunders Co. 1989); The Cecil Textbook of Medicine, 19th Ed. (W. B. 1992); and Sabiston, D, The Textbook of Surgery, 14th Ed. (W. B. 1991). Additional references describing cell types found in the blood vessels, and those of the vasculature which may be useful in the methods of the present invention include the following: W. Bloom & D. Fawcett, A Textbook of Histology (V.B. Saunders Co. 1975).

Various publications have postulated on the uses of gene transfer for the prevention of disease, including heart disease. See, e.g., Methods in Virology, Vol. 7: Gene Transfer and Expression Protocols, Murray, E. (ed.), Weiss, Clifton, N.J., 1991; Mazur et al., Molecular and Cellular Biology, 21:104-111, 1994; French, Herz 18:222-229, 1993; Williams, Journal of Medical Sciences 306:129-136, 1993; and Schneider, Circulation 88:1937-1942, 1993.

The references cited in the above section are hereby incorporated by reference herein to the extent that these references teach techniques that are employed in the practice of the present invention.

### INCORPORATION BY REFERENCE

All references cited within this application, including patents, patent applications and other publications, are hereby incorporated by reference.

### DETAILED DESCRIPTION OF VARIOUS PREFERRED EMBODIMENTS

Various preferred aspects of the present invention are summarized below and further described and illustrated in the subsequent detailed descriptions and figures.

The present invention relates to methods and compositions for treating cardiovascular diseases including myocardial ischemia, heart failure and peripheral vascular disease.

In the present method, for treating heart disease, a vector construct containing a gene encoding an angiogenic protein or peptide is targeted to the heart of a patient whereby the exogenous angiogenic protein is expressed in the myocardium, thus ameliorating cardiac dysfunction by improving blood flow and/or improving cardiac contractile function. Improved heart function ultimately leads to the reduction or disappearance of one or more symptoms of heart disease or heart failure and prolonged life beyond the expected mortality.

Similarly, in the treatment of peripheral vascular disease according to the present method, a vector construct comprising a transgene encoding at least one angiogenic protein or peptide is targeted to the affected tissue, for example ischemic skeletal muscle, whereby synthesis of the exogenous angiogenic protein ameliorates and/or cures symptoms of the peripheral vascular disease, for example by increasing blood flow to the affected (e.g., ischemic) region of the tissue and/or, in muscle, by improving contractile function of the affected muscle.

Thus, in a preferred aspect, the present invention provides a method for treating heart disease in a patient having myocardial ischemia, comprising delivering a transgene-inserted vector to the myocardium of the patient by intracoronary injection, preferably by injecting the vector directly into one or both coronary arteries (or grafts), whereby the transgene is expressed and blood flow and/or contractile function are improved. By way of illustration, using a vector comprising a transgene coding for an angiogenic protein or peptide, such as, for example, FGF-5, FGF-4, aFGF, bFGF and/or a VEGF, which vector is delivered to the heart where the protein or peptide is produced to a therapeutically significant degree in the myocardium continuously for sustained periods, angiogenesis can be promoted in the affected region of the myocardium. Other transgenes, such as those encoding beta-adrenergic signaling proteins or other cardiac- or muscle-enhancing proteins, can also be used, as described below, in conjunction with the use of an angiogenic transgene. The vectors employed in the invention can be a plasmid or preferably a viral vector, for example a replication-deficient adenovirus or adeno-associated virus (AAV). By injecting the viral vector stock, such as one that contains relatively few or no wild-type virus, deeply into the lumen of one or both coronary arteries (or grafts), preferably into both the right and left coronary arteries (or grafts), and preferably in an amount of about 10⁷-10¹³ viral particles as determined by optical densitometry (more preferably about 10⁹-10¹¹ viral particles), it is possible to locally transfect a desired number of cells in the affected myocardium with angiogenic protein- or peptide-encoding genes, thereby maximizing therapeutic efficacy of gene transfer, and minimizing both undesirable angiogenesis at extracardiac sites and the possibility of an inflammatory response to viral proteins.

In another preferred aspect, the present invention can also be used to treat a patient suffering from congestive heart failure, by delivering a transgene-inserted vector to the heart of said patient, the vector comprising a transgene encoding an angiogenic protein or peptide, whereby the transgene is expressed in the myocardium resulting in increased blood flow and function in the heart. Among such patients suffering from congestive heart failure are those exhibiting dilated cardiomyopathy and those who have exhibited severe myocardial infarctions, typically associated with severe or occlusive coronary artery disease. The vector is preferably introduced into a blood vessel supplying blood to the myocardium of the heart, so as to deliver the vector to the myocardium. Preferably the vector is introduced into the lumen of a coronary artery, a saphenous vein graft, or an internal mammary artery graft; most preferably, the vector is introduced into the lumen of both a left and right coronary artery. The intracoronary injection is preferably made, as a single injection, relatively deeply within each of the selected artery(s), (e.g., preferably at least about 1 cm into the lumens of the vessel(s)).

The techniques of the present invention are also useful to prevent or alleviate deleterious ventricular remodeling in a patient who has suffered (or may suffer) a myocardial infarction. Again, a vector comprising a transgene encoding an angiogenic protein or peptide, preferably operably linked to a promoter for expression of the gene, is delivered to the heart of the patient, where the transgene is expressed and the deleterious ventricular remodeling alleviated.

Transgenes Encoding Angiogenic Proteins and Peptides

In the present invention, one or more transgenes encoding an angiogenic protein or peptide factor that can enhance blood flow and/or contractile function can be used. Any protein or peptide that exhibits angiogenic activity, measurable by the methods described herein and in the art, can be potentially employed in connection with the present invention. A number of such angiogenic proteins are known in the art and new forms are routinely identified. Suitable angiogenic proteins or peptides are exemplified by members of the family of fibroblast growth factors (FGF), vascular endothelial growth factors (VEGF), platelet-derived growth factors (PDGF), insulin-like growth factors (IGF), and others. Members of the FGF family include, but are not limited to, aFGF (FGF-1), bFGF (FGF-2), FGF-4 (also known as “hst/KS3”), FGF-5, FGF-6. VEGF has been shown to be expressed by cardiac myocytes in response to ischemia in vitro and in vivo; it is a regulator of angiogenesis under physiological conditions as well as during the adaptive response to pathological states (Banai et al. Circulation 89:2183-2189, 1994). The VEGF family, includes, but is not limited to, members of the VEGF-A sub-family (e.g. VEGF-121, VEGF-145, VEGF-165, VEGF-189 and VEGF-206), as well as members of the VEGF-B sub-family (e.g. VEGF-167 and VEGF-186) and the VEGF-C sub-family. PDGF includes, e.g., PDGF A and PDGF B, and IGF includes, for example, IGF-1. Other angiogenic proteins or peptides are known in the art and new ones are regularly identified. The nucleotide sequences of genes encoding these and other proteins, and the corresponding amino acid sequences are likewise known in the art (see, e.g., the GENBANK sequence database).

Angiogenic proteins and peptides include peptide precursors that are post-translationally processed into active peptides and “derivatives” and “functional equivalents” of angiogenic proteins or peptides. Derivatives of an angiogenic protein or peptide are peptides having similar amino acid sequence and retaining, to some extent, one or more activities of the related angiogenic protein or peptide. As is well known to those of skill in the art, useful derivatives generally have substantial sequence similarity (at the amino acid level) in regions or domains of the protein associated with the angiogenic activity. Similarly, those of skill in the art will readily appreciate that by “functional equivalent” is meant a protein or peptide that has an activity that can substitute for one or more activities of a particular angiogenic protein or peptide. Preferred functional equivalents retain all of the activities of a particular angiogenic protein or peptide; however, the functional equivalent may have an activity that, when measured quantitatively, is stronger or weaker than the wild-type peptide or protein.

For details on the FGF family, see, e.g., Burgess, Ann. N.Y. Acad. Sci. 638: 89-97, 1991; Burgess et al. Annu. Rev. Biochem. 58: 575-606, 1989; Muhlhauser et al., Hum. Gene Ther. 6: 1457-1465, 1995; Zhan et al., Mol. Cell. Biol., 8: 3487, 1988; Seddon et al., Ann. N.Y. Acad. Sci. 638: 98-108, 1991. For human hst/KS3 (i.e. FGF-4), see Taira et al. Proc. Natl. Acad. Sci. USA 84: 2980-2984, 1987. For human VEGF-A protein, see e.g., Tischer et al. J. Biol. Chem. 206: 11947-11954, 1991, and references therein; Muhlhauser et al., Circ. Res. 77: 1077-1086, 1995; and Neufeld et al., WO 98/10071 (12 Mar. 1998). Other variants of known angiogenic proteins have likewise been described; for example variants of VEGF proteins and VEGF related proteins, see e.g., Baird et al., WO 99/40197, (12 Aug. 1999); and Bohlen et al., WO 98/49300, (5 Nov. 1998). Combinations of angiogenic proteins and gene delivery vectors encoding such combinations are described in Gao et al. U.S. Ser. No. 09/607,766, filed 30 Jun. 2000, entitled “Dual Recombinant Gene Therapy Compositions and Methods of Use”, hereby incorporated by reference in its entirety. As is also appreciated by those of skill in the art, angiogenic proteins can promote angiogenesis by enhancing the expression, stability or functionality of other angiogenic proteins. Examples of such angiogenic proteins or peptides include, e.g., regulatory factors that are induced in response to hypoxia (e.g. the hypoxia-inducible factors such as Hif-1, Hif-2 and the like; see, e.g., Wang et al., Proc. Natl. Acad. Sci. USA 90(9): 4304-8, 1993; Forsythe et al., Mol. Cell. Biol. 16(9): 4604-13, 1996; Semenza et al., Kidney Int., 51(2): 553-5, 1997; and O'Rourke et al., Oncol. Res., 9(6-7): 327-32, 1997; as well as other regulatory factors, such as, for example, those that are induced by physiological conditions associated with cardiovascular disease, such as inflammation (e.g., inducible nitric oxide synthase (iNOS), as well as the constitutive counterpart, cNOS; see e.g., Yoshizumi et al., Circ. Res., 73(1): 205-9, 1993; Chartrain et al., J. Biol. Chem., 269(9): 6765-72, 1994; Papapetropoulos et al., Am. J. Pathol., 150(5): 1835-44, 1997; and Palmer, et al., Am. J. Physiol., 274(2 Pt 1): L212-9, 1998). Additional examples of such angiogenic proteins include certain insulin-like growth factors (e.g., IGF-1) and angiopoietins (Angs), which have been reported to promote and/or stimulate expression and/or activity of other angiogenic proteins such as VEGF (see e.g. Goad, et al, Endocrinology, 137(6):2262-68 (1996); Warren, et al., J. Bio. Chem., 271(46):29483-88 (1996); Punglia, et al, Diabetes, 46(10):1619-26 (1997); and Asahara, et al., Circ. Res., 83(3):233-40 (1998) and Bermont et al. Int. J. Cancer 85: 117-123, 2000). Similarly, hepatocyte growth factor (also referred to as Scatter factor), which has been reported to induce blood vessel formation in vivo (see, e.g., Grant et al. Proc. Natl. Acad. Sci. USA 90: 1937-1941, 1993) has also been reported to increase expression of VEGF (see, e.g., Wojta et al., Lab Invest. 79:427-438, 1999). Additional examples of angiogenic polypeptides include natural and synthetic regulatory peptides (angiogenic polypeptide regulators) that act as promoters of endogenous angiogenic genes. Native angiogenic polypeptide regulators can be derived from inducers of endogenous angiogenic genes. Hif, as described above, is one illustrative example of such an angiogenic gene which has been reported to promote angiogenesis by inducing expression of other angiogenic genes. Synthetic angiogenic polypeptide regulators can be designed, for example, by preparing multi-finger zinc-binding proteins that specifically bind to sequences upstream of the coding regions of endogenous angiogenic genes and which can be used to induce the expression of such endogenous genes. Studies of numerous genes has led to the development of “rules” for the design of such zinc-finger DNA binding proteins (see, e.g., Rhodes and Klug, Scientific American, February 1993, pp 56-65; Choo and Klug, Proc. Natl. Acad. Sci. USA, 91(23): 11163-7, 1994; Rebar and Pabo, Science, 263(5147): 671-3, 1994; Choo et al., J. Mol. Biol., 273(3): 525-32, 1997; Pomerantz et al., Science 267: 93-96, 1995; and Liu et al., Proc. Natl. Acad. Sci. USA, 94: 5525-5530, 1997. As will be appreciated by those of skill in the art, numerous additional genes encoding proteins or peptides having the capacity to directly or indirectly promote angiogenesis are regularly identified and new genes will be identified based on similarities to known angiogenic protein or peptide encoding genes or to the discovered capability of such genes to encode proteins or peptides that promote angiogenesis. Sequence information for such genes and encoded polypeptides is readily obtainable from sequence databases such as GenBank or EMBL. Polynucleotides encoding these proteins can also be obtained from gene libraries, e.g., by using PCR or hybridization techniques routine in the art.

Preferably, the angiogenic protein-encoding transgene is operably linked to a promoter that directs transcription and expression of the gene in a mammalian cell, such as a cell in the heart or in the skeletal muscle. One presently preferred promoter is a CMV promoter. In other preferred embodiments, as discussed further below, the promoter is a tissue-specific promoter, such as a cardiac-specific promoter (e.g., a cardiomyocyte-specific promoter). Preferably, the gene encoding the angiogenic factor is also operably linked to a polyadenylation signal.

Success of the gene transfer approach requires both synthesis of the gene product and secretion from the transfected cell. Thus, preferred angiogenic proteins or peptides include those which are naturally secreted or have been modified to permit secretion, such as by operably linking to a signal peptide. From this point of view, a gene encoding a secreted angiogenic protein, such as, FGF-4, FGF-5, or FGF-6 is preferred since these proteins contain functional secretory signal sequences and are readily secreted from cells. Many if not most human VEGF proteins (including but not limited to VEGF-121 and VEGF-165) also are readily secreted and diffusible after secretion. Thus, when expressed, these angiogenic proteins can readily access the cardiac interstitium and induce angiogenesis. Blood vessels that develop in angiogenesis include capillaries which are the smallest caliber blood vessels having a diameter of about 8 microns, and larger caliber blood vessels that have a diameter of at least about 10 microns. Angiogenic activity can be determined by measuring blood flow, increase in function of the treated tissue or the presence of blood vessels, using procedures known in the art or described herein. For example, capillary number or density can be quantitated in an animal visually or by microscopic analysis of the tissue site (see Example 5).

With other angiogenic proteins such as aFGF (FGF-1) and bFGF (FGF-2) that lack a native secretory signal sequence, fusion proteins having secretory signal sequences can be recombinantly produced using standard recombinant DNA methodology familiar to one of skill in the art. It is believed that both aFGF and bFGF are naturally secreted to some degree; however, inclusion of an additional secretion signal sequence can be used to enhance secretion of the protein. The secretory signal sequence would typically be positioned at the N-terminus of the desired protein but can be placed at any position suitable to allow secretion of the angiogenic factor. For example, a polynucleotide containing a suitable signal sequence can be fused 5′ to the first codon of the selected angiogenic protein gene. Suitable secretory signal sequences include signal sequences of the FGF-4, FGF-5, FGF-6 genes or a signal sequence of a different secreted protein such as IL-1-beta. Example 7 below exemplifies one type of modification of an angiogenic protein to contain a signal sequence from another protein, the modification achieved by replacement of residues in the angiogenic protein with residues that direct secretion of the secreted second protein. A signal sequence derived from a protein that is normally secreted from cardiac myocytes can be used. Angiogenic genes can also provide additional functions that can improve, for example cardiac cell function. For example, FGFs can provide cardiac enhancing and/or “ischemic protectant effects” that may be independent of their capability to promote angiogenesis. Thus, angiogenic genes can be used to enhance cardiac function by mechanisms that are additional to or in place of the promotion of angiogenesis per se. As an additional example, IGFs, which can promote angiogenesis, can also enhance muscle cell function (see e.g. Musaro et al. Nature 400: 581-585, 1999); as well as exhibit anti-apoptotic effects (see e.g. Lee et al. Endocrinology 140: 4831-4840, 1999). Other proteins which enhance muscle cell function can also be employed in accordance with the methods of the present invention.

As noted above, genes encoding one or more angiogenic proteins or peptides can be used in conjunction with the present invention. Thus, a gene or genes encoding a combination of angiogenic proteins or peptides can be delivered using one or more vectors according to the methods described herein. The families of angiogenic genes described herein and in the art comprise numerous examples of such genes. Preferably, where such a combination is employed, the genes may be derived from different families of angiogenic factors (such as a combination selected from two or more different members of the group consisting of FGFs, VEGFs, PDGFs and IGFs). To take a single illustration of such a combination, a vector comprising an FGF gene and a VEGF gene may be used. As an illustrative example, we have used a combination of an FGF gene (FGF-4 fragment 140) (see e.g., the FGF-4 gene and variants thereof described by Basilico et al., in U.S. Pat. No. 5,459,250, issued 17 Oct. 1995, and related cases) and a variant VEGF gene (VEGF-145 mutein 2) (see, e.g., the VEGF-145 gene and variants thereof described by Neufeld et al., WO 98/10071, published 12 Mar. 1998, and related cases). Such combinations can exhibit additive and/or synergistic effects. Numerous other combinations will be apparent to those of skill in the art based on these teachings. Vectors comprising angiogenic genes or combinations of angiogenic genes, in accordance with the present invention, can also include one or more other genes that can be used to further enhance tissue blood flow and/or contractile function. In the heart, for example, genes encoding beta-ASPs (as described, by Hammond et al., in co-pending applications WO 98/10085, published 12 Mar. 1998) can be employed in combination with one or more genes encoding angiogenic proteins or peptides. Other cardiac or muscle cell enhancing proteins can similarly be incorporated into the compositions and methods of the present invention.

Combinations of genes that can be employed in accordance with the present invention can be provided within a single vector (e.g., as separate genes, each under the control of a promoter, or as a single transcriptional or translational fusion gene). Combinations of genes can also be provided as a combination of vectors (which may be derived from the same or different vectors, such as a combination of adenovirus vectors, or an adenovirus vector and an AAV vector); which can be introduced to a patient coincidentally or in series. In the case of Adenovirus (Ad) and Adeno-associated virus (AAV), the presence of Ad, which is normally a helper virus for AAV, can enhance the ability of AAV to mediate gene transfer. An Ad vector may thus be introduced coincident with or prior to introduction of an AAV vector according to the present invention. In addition to transfection efficiency, the choice of vector is also influenced by the desired longevity of transgene expression. By way of illustration, since many angiogenic genes can bring about long-term effects without requiring long-term expression (e.g., by initiating or facilitating the process of angiogenesis which results in an increase in tissue vascularization), angiogenic genes may be introduced using an adenovirus (or other vector that does not normally integrate into host DNA) which might be used prior to or in combination with the introduction of an AAV vector carrying a transgene for which longer-term expression is desired (e.g., a beta-ASP transgene). Other combinations of transgenes and/or vectors will be apparent to those of skill in the art based on the teachings and illustrations of the present invention.

For treating humans, genes encoding angiogenic proteins of human origin are preferred although angiogenic proteins of other mammalian origin that exhibit cross-species activity i.e. having angiogenic activity in humans, can also be used.

Vectors for Gene Delivery in Vivo

In general, the gene of interest is transferred to the heart or to the peripheral vasculature in vivo, and directs production of the encoded protein. Preferably such production is constitutive (although inducible expression systems can also be employed).

Vectors useful in the present invention include viral vectors, lipid-based vectors (e.g., liposomes) and other vectors that are capable of delivering DNA to non-dividing cells in vivo. Presently preferred are viral vectors, particularly replication-defective viral vectors including, for example, replication-defective adenovirus vectors and adeno-associated virus vectors. For ease of production and use in the present invention, replication-defective adenovirus vectors are presently most preferred. Adenovirus efficiently infects non-dividing cells and is therefore useful for expressing recombinant genes in the myocardium because of the nonreplicative nature of cardiac myocytes.

A variety of other vectors suitable for in vivo gene therapy can readily be employed to deliver angiogenic protein transgenes for use in the present invention. Such other vectors include other viral vectors (such as AAV), non-viral protein-based delivery platforms, as well as lipid-based vectors (such as liposomes, micelles, lipid-containing emulsions and others that have been described in the art). With respect to AAV vectors, as is known in the art, they are preferably replication-defective in humans, such as for example, having the rep and cap genes removed (which sequence must therefore be supplied in trans to replicate and package AAV vectors, typically in a packaging cell line) and the inserted transgene (including, for example, a promoter operably linked thereto) is preferably flanked by AAV inverted terminal repeats (ITRs).

Recombinant viral vectors comprise one or more heterologous genes or sequences. Since many viral vectors exhibit size-constraints associated with packaging, and since replication-deficient viral vectors are generally preferred for in vivo delivery, the heterologous genes or sequences are typically introduced by replacing one or more portions of the viral genome. Such viruses may become replication-deficient, as a result of the deletions, thereby requiring the deleted function(s) to be provided in trans during viral replication and encapsidation (by using, e.g., a helper virus or a packaging cell line carrying genes necessary for replication and/or encapsidation) (see, e.g., the references and illustrations below). As stated above, modified AAV vectors in which transgenes are inserted in place of viral rep and/or cap genes are likewise well known in the art. Similarly, modified viral vectors in which a polynucleotide to be delivered is carried on the outside of the viral particle have also been described (see, e.g., Curiel, D T, et al. PNAS 88:8850-8854, 1991). References describing a these and other gene delivery vectors are known in the art, a number of which are cited herein.

As described above and in the cited references, vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence processing and/or localization of the vector and its nucleic acid within the cell after uptake (such as agents mediating intracellular processing and/or nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A detectable marker gene allows cells carrying the gene to be specifically detected (e.g., distinguished from cells which do not carry the marker gene). One example of such a detectable marker gene is the lacZ gene, encoding beta-galactosidase, which allows cells transduced with a vector carrying the lacZ gene to be detected by staining, as described below. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., Lupton, S., WO 92/08796, published 29 May 1992; and Lupton, S., WO 94/28143, published 8 Dec. 1994). Such marker genes can provide an added measure of control that can be advantageous in gene therapy contexts. A large variety of such vectors are known in the art and are generally available (see, e.g., the various references cited above).

References describing adenovirus vectors and other viral vectors which could be used in the methods of the present invention include the following: Horwitz, M. S., Adenoviridae and Their Replication, in Fields, B., et al. (eds.) Virology, Vol. 2, Raven Press New York, pp. 1679-1721, 1990); Graham, F., et al., pp. 109128 in Methods in Molecular Biology, Vol. 7: Gene Transfer and Expression Protocols, Murray, E. (ed.), Humana Press, Clifton, N.J. (1991); Miller, N., et al., FASEB Journal 9: 190-199, 1995; Schreier, H, Pharmaceutica Acta Helvetiae 68: 145-159, 1994; Schneider and French, Circulation 88:1937-1942, 1993; Curiel D. T., et al., Human Gene Therapy 3: 147-154, 1992; Graham, F. L., et al., WO 95/00655 (5 Jan. 1995); Falck-Pedersen, E. S., WO 95/16772 (22 Jun. 1995); Denefle, P. et al., WO 95/23867 (8 Sep. 1995); Haddada, H. et al., WO 94/26914 (24 Nov. 1994); Perricaudet, M. et al., WO 95/02697 (26 Jan. 1995); Zhang, W., et al., WO 95/25071 (12 Oct. 1995). A variety of adenovirus plasmids are also available from commercial sources, including, e.g., Microbix Biosystems of Toronto, Ontario (see, e.g., Microbix Product Information Sheet: Plasmids for Adenovirus Vector Construction, 1996). Various additional adenoviral vectors and methods for their production and purification are regularly identified.

Additional references describing AAV vectors which could be used in the methods of the present invention include the following: Carter, B., Handbook of Parvoviruses, vol. 1, pp. 169-228, 1990; Berns, Virology, pp. 1743-1764 (Raven Press 1990); Carter, B., Curr. Opin. Biotechnol., 3: 533-539, 1992; Muzyczka, N., Current Topics in Microbiology and Immunology, 158: 92-129, 1992; Flotte, T. R., et al., Am. J. Respir. Cell Mol. Biol. 7:349-356, 1992; Chatterjee et al., Ann. NY Acad. Sci., 770: 79-90, 1995; Flotte, T. R., et al., WO 95/13365 (18 May 1995); Trempe, J. P., et al., WO 95/13392 (18 May 1995); Kotin, R., Human Gene Therapy, 5: 793-801, 1994; Kotin et al., WO 98/11244 (19 Mar. 1998); Kotin et al., WO 99/61601 (2 Dec. 1999); Flotte, T. R., et al., Gene Therapy 2:357-362, 1995; Allen, J. M., WO 96/17947 (13 Jun. 1996); and Du et al., Gene Therapy 3: 254261, 1996. Various additional AAV vectors and methods for their production and purification are regularly identified.

As described above and in the scientific literature, a number of retrovirus-derived systems have also been developed to be used in in vivo gene delivery. By way of illustration, the lentivirus genus of retroviruses (for example, human immunodeficiency virus, feline immunodeficiency virus and the like) can be modified so that they are able to transduce cells that are typically non-dividing (see, e.g., Poeschla et al., PNAS 96:11395-11399, 1996; Naldini et al., PNAS 96:11382-11388, 1996; Naldini et al., Science 272:263-267, 1996; Srinivasakumar et al., J. Virol. 71: 5841-5848, 1997; Zufferey et al., Nat. Biotechnol. 15: 871 -875, 1997; Kim et al., J. Virol. 72: 811-816, 1998; Miyoshi et al., J. Virol. 72:8150-8157, 1998; see also Buchschacher et al., Blood 15:2499-2504, 2000; see also The Salk Institute, WO97/12622 (10 Apr. 1997)). While HIV-based lentiviral vector systems have received some degree of focus in this regard, other lentiviral systems have recently been developed, such as feline immunodeficiency virus-based lentivirus vector systems, that offer potential advantages over the HIV-based systems (see e.g. Poeschla et al., Nat. Med. 4:354-357, 1998; Johnston et al., J. Virol. 73: 2491-2498, 1999; and Johnston et al., J. Virol. 73: 4991-5000, 1999; see also the review by Romano et al., Stem Cells 18:19-39, 2000 and references reviewed therein).

In addition to viral vectors, non-viral vectors that may be employed as a gene delivery means are likewise known and continue to be developed. For example, non-viral protein-based delivery platforms, such as macromolecular complexes comprising a DNA binding protein and a carrier or moiety capable of mediating gene delivery, as well as lipid-based vectors (such as liposomes, micelles, lipid-containing emulsions and others) have been described in the art. References describing non-viral vectors which could be used in the methods of the present invention include the following: Ledley, F D, Human Gene Therapy 6: 11 29-1144, 1995; Miller, N., et al., FASEB Journal 9: 190-199, 1995; Chonn, A., et al., Curr. Opin. in Biotech. 6: 698-708, 1995; Schofield, J P, et al., British Med. Bull. 51: 56-71, 1995; Brigham, K. L., et al., J. Liposome Res. 3: 31 49, 1993; Brigham, K. L., WO 91/06309 (16 May 1991); Felgner, P. L., et al., WO 91/17424 (14 Nov. 1991); Solodin et al., Biochemistry 34: 13537-13544, 1995; WO 93/19768 (14 Oct. 1993); Debs et al., WO 93/125673; Felgner, P. L., et al., U.S. Pat. No. 5,264,618 (Nov. 23, 1993); Epand, R. M., et al., U.S. Pat. No. 5,283,185 (Feb. 1, 1994); Gao et al., WO 96/22765 (1 Aug. 1996); Gebeyehu et al., U.S. Pat. No. 5,334,761 (Aug. 2, 1994); Felgner, P. L., et al., U.S. Pat. No. 5,459,127 (Oct. 17, 1995); Overell, R. W., et al., WO 95/28494 (26 Oct. 1995); Jessee, WO 95/02698 (26 Jan. 1995); Haces and Ciccarone, WO 95/17373 (29 Jun. 1995); Lin et al., WO 96/01840 (25 Jan. 1996). Numerous additional lipid-mediated in vivo gene delivery vectors and vector delivery co-factors have been identified (see e.g. Kollen et al., Hum. Gene Ther. 10:615-22, 1999; Roy et al., Nat. Med. 5:387-391; Fajac et al., Hum. Gene Ther. 10:395-406, 1999; Ochiya et al., Nat. Med. 5:707-710, 1999). Additionally, the development of systems which combine components of viral and non-viral mediated gene delivery systems have been described and may be employed herein (see e.g. Philip et al., Mol. Cell Biol., 14: 2411-2418, 1994; see also Di Nicola et al., Hum. Gene Ther. 10:1875-1884, 1999). Various additional non-viral gene delivery vectors and methods for their preparation and purification are regularly identified.

As described above, the efficiency of gene delivery using a vector such as a viral vector can be enhanced by delivering the vector into a blood vessel such as an artery or into a tissue that is pre-infused and/or co-infused with a vasoactive agent, for example histamine or a histamine agonist, or a vascular endothelial growth factor (VEGF) protein, as described herein and further illustrated in co-pending PCT application WO 99/40945, published 19 Aug. 1999. Another example of a vasoactive agent that can be employed to enhance the efficiency of gene delivery is a nitric oxide donor such as sodium nitroprusside. Most preferably the vasoactive agent is infused into the blood vessel or tissue coincidently with and/or within several minutes prior to introduction of the vector. Vasoactive agent, as used herein, refers to a natural or synthetic substance that induces increased vascular permeability and/or enhances transfer of macromolecules such as gene delivery vectors from blood vessels, e.g. across capillary endothelia. By augmenting vascular permeability to macromolecules or otherwise facilitating the transfer of macromolecules into the capillary bed perfused by an artery, vasoactive agents can enhance delivery of these vectors to the targeted sites and thus effectively enhance overall expression of the transgene in the target tissue. We have employed histamine as a vasoactive agent and such was found to substantially enhance delivery of a vector to an infused site such as the myocardium. Histamine derivatives and agonists, such as those that interact with histamine H receptors, which can be employed include, for example, 2-methylhistamine, 2-pyridylethylamine, betahistine, and 2 thiazolylethylamine. These and additional histamine agonists are described, for example, in Garrison J C., Goodman and Gilman's The Pharmacological Basis of Therapeutics (8th Ed: Gilman A G, Rall T W, Nies A S, Taylor P, eds) Pergamon Press, 1990, pp 575-582 and in other pharmacological treatises. In addition to histamine and histamine agonists, which can be employed as vasoactive agents, vascular endothelial growth factors (VEGFs) and VEGF agonists (as described herein and in the cited references) can also induce increased vascular permeability and can therefore be used as a vasoactive agent to enhance gene delivery in the context of the compositions and methods described herein. As with histamine, the VEGF is preferably infused into a blood vessel supplying the target site over several minutes prior to infusion of vector. Nitric oxide donors, such as sodium nitroprusside (SNP), can also be employed as vasoactive agents. Preferably the nitric oxide donor (e.g., SNP) is pre-infused into the target tissue (or blood vessel supplying a target tissue), beginning several minutes prior to and continuing up until the time of infusion of the vector composition. Administration can also be continued during infusion of the vector composition.

An Exemplary Adenoviral Vector that is Helper-Independent and Replication-Deficient in Humans

In general, the gene of interest is transferred to the heart or to the peripheral vasculature, in vivo, and directs production of the encoded protein. Several different gene transfer approaches are feasible. Presently preferred is a helper-independent replication-deficient system based on human adenovirus 5 (Ad5). Using a single intracoronary injection of such a recombinant Ad5-based system, we have demonstrated significant transfection of myocardial cells in vivo (Giordano and Hammond, Clin. Res., 42:123A, 1994). Non-replicative recombinant adenoviral vectors are particularly useful in transfecting coronary endothelium and cardiac myocytes resulting in highly efficient transfection after intracoronary injection. Adenovirus vectors can also be used to transfect tissue supplied by the peripheral vasculature, e.g., by intra-arterial or direct injection.

As demonstrated herein, the helper-independent replication-defective human adenovirus 5 system can be used to effectively transfect a large percentage of myocardial cells in vivo by a single intracoronary injection. We have also shown that such a delivery technique can be used to effectively target vectors to the myocardium of a large mammal heart. Additional means of targeting vectors to particular cells or tissue types are described below and in the art.

In various illustrations described below, the recombinant adenovirus vectors used are based on the human adenovirus 5 (as described by McGrory W J et al., Virology 163:614-617, 1988) which are missing essential early genes from the adenovirus genome (usually E1A/E1B), and are therefore unable to replicate unless grown in permissive cell lines that provide the missing gene products in trans. In place of the missing adenovirus genomic sequences, a transgene of interest can be cloned and expressed in tissue/cells infected with the replication-defective adenovirus. Generally, adenovirus-based gene transfer does not result in stable integration into the target cell genome. However, adenovirus vectors can be propagated in high titer and transfect non-replicating cells; and, although the transgene is not passed to daughter cells, this is suitable for gene transfer to adult cardiac myocytes, which do not actively divide. Retrovirus vectors provide stable gene transfer, and high titers are now obtainable via retrovirus pseudotyping (Burns, et al., Proc Natl. Acad. Sci. USA, 90: 8033-8037, 1993), but current retrovirus vectors are generally unable to efficiently transduce nonreplicating cells (e.g., cardiac myocytes) efficiently. In addition, the potential hazards of transgene incorporation into host DNA are not warranted if short-term gene transfer is sufficient.

Indeed, we have demonstrated that a limited duration in the expression of an angiogenic protein is sufficient to substantially improve blood flow and function in the ischemic tissue (see Example 5). Thus, transient gene transfer is therapeutically adequate for treating such cardiovascular conditions. Within 14 days after gene transfer of FGF-5 into the myocardium, blood flow to the ischemic bed had increased two-fold and the effect persisted for at least 12 weeks (Example 5 and FIG. 8). The increased blood flow correlated with an increase in the number of capillaries in the heart (see Example 5). Wall thickening also increased within two weeks after gene transfer and persisted for at least 12 weeks. Thus, the angiogenic factor gene does not have to be present in the infected cell for more than a few weeks to produce a therapeutic effect. Once the blood vessels have developed, continued expression of the exogenous angiogenic protein may not be required to maintain the new vascular structure and increased blood flow.

An advantage associated with non-dividing cells such as myocytes is that the viral vector is not readily “diluted out” by host cell division. However, if it is necessary or desirable to further enhance duration of transgene expression in the heart, it is also possible to employ various second generation adenovirus vectors that have both E1 and E4 deletions, which can be used in conjunction with cyclophosphamide administration (See, e.g., Dai et al., Proc. Natl. Acad. Sci. USA, 92: 1401-1405, 1995).

Human 293 cells (Accession No. ATCC CRL1573; Rockville, Md.), which are human embryonic kidney cells transformed with adenovirus E1A /E1 B genes, typify useful permissive cell lines for the production of such replication-defective vectors. However, other cell lines which allow replication-defective adenovirus vectors to propagate therein can also be used, such as HeLa cells.

Construction of Recombinant Adenoviral Vector

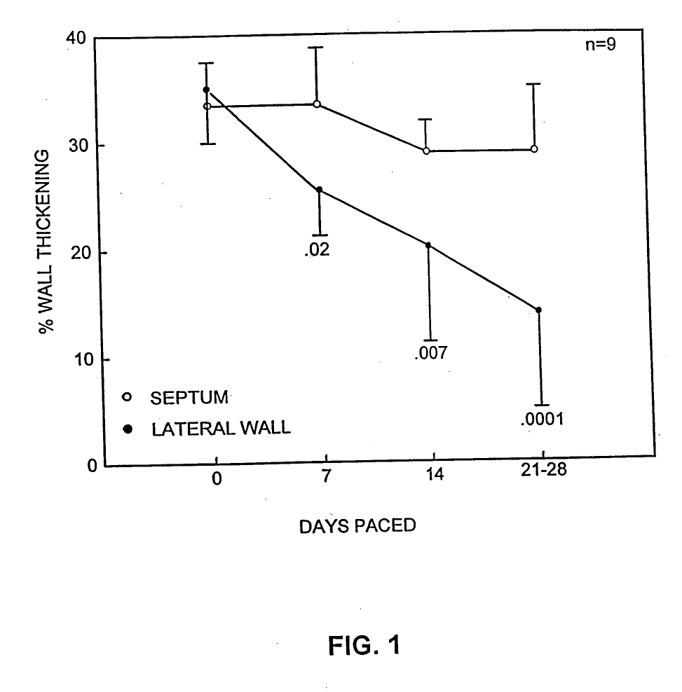

Adenoviral vectors used in the present invention can be constructed by the rescue recombination technique described in Graham, Virology 163:614-617, 1988. Briefly, the transgene of interest is cloned into a shuttle vector that contains a promoter, polylinker and partial flanking adenoviral sequences from which E1A/E1B genes have been deleted. As the shuttle vector, plasmid pAC1 (Virology 163:614-617, 1988) (or an analog) which encodes portions of the left end of the human adenovirus 5 genome (Virology 163:614-617, 1988) minus the early protein encoding E1A and E1 B sequences that are essential for viral replication, and plasmid ACCMVPLPA (Gomez-Foix et al., J. Biol. Chem. 267: 25129-25134, 1992) which contains polylinker, the CMV promoter and SV40 polyadenylation signal flanked by partial adenoviral sequences from which the E1A /E1B genes have been deleted can be exemplified. The use of plasmid pAC1 or ACCMVPLA facilitates the cloning process. The shuttle vector is then co-transfected with a plasmid which contains the entire human adenoviral 5 genome with a length too large to be encapsulated, into 293 cells. Co-transfection can be conducted by calcium phosphate precipitation or lipofection (Zhang et al., Biotechniques 15:868-872, 1993). Plasmid JM17 encodes the entire human adenovirus 5 genome plus portions of the vector pBR322 including the gene for ampicillin resistance (4.3 kb). Although JM17 encodes all of the adenoviral proteins necessary to make mature viral particles, it is too large to be encapsulated (40 kb versus 36 kb for wild type). In a small subset of co-transfected cells, rescue recombination between the transgene containing the shuttle vector such as plasmid pAC1 and the plasmid having the entire adenoviral 5 genome such as plasmid pJM17 provides a recombinant genome that is deficient in the E1A/E1B sequences, and that contains the transgene of interest but secondarily loses the additional sequence such as the pBR322 sequences during recombination, thereby being small enough to be encapsulated (see FIG. 1). With respect to the above method, we have reported successful results (Giordano, et al. Circulation 88:1-139, 1993, and Giordano. and Hammond, Clin. Res. 42:123A, 1994). The CMV driven β-galactosidase encoding adenovirus HCMVSP1lacZ (Clin Res 42:123A, 1994) can be used to evaluate efficiency of gene transfer using X-gal treatment.

Targeted Vector Constructs

Limiting expression of the angiogenic transgene to the heart, or to particular cell types within the heart (e.g. cardiac myocytes) or to other target tissues, such as those in the peripheral vasculature, can provide certain advantages as discussed below.

The present invention contemplates the use of targeting not only by delivery of the transgene into the coronary artery or other tissue-specific conduit, for example, but also by use of targeted vector constructs having features that tend to target gene delivery and/or gene expression to particular host cells or host cell types (e.g. cardiac or other myocytes). Such targeted vector constructs would thus include targeted delivery vectors and/or targeted vectors, as described in more detail below and in the published art. Restricting delivery and/or expression can be beneficial as a means of further focusing the potential effects of gene therapy. The potential usefulness of further restricting delivery/expression depends in large part on the type of vector being used and the method and place of introduction of such vector. As described herein, delivery of viral vectors via intracoronary injection to the myocardium has been observed to provide, in itself, highly targeted gene delivery (see the Examples below). In addition, using vectors that generally do not result in transgene integration into a replicon of the host cell (such as adenovirus and numerous other vectors), cardiac myocytes are expected to exhibit relatively long transgene expression since the cells do not generally replicate. In contrast, expression in rapidly dividing cells such as endothelial cells would tend to be decreased by cell division and turnover. However, other means of limiting delivery and/or expression can also be employed, in addition to or in place of the illustrated delivery methods, as described herein.

Targeted delivery vectors include, for example, vectors (such as viruses, non-viral protein-based vectors and lipid-based vectors) having surface components (such as a member of a ligand-receptor pair, the other half of which is found on a host cell to be targeted) or other features that mediate preferential binding and/or gene delivery to particular host cells or host cell types. As is known in the art, a number of vectors of both viral and non-viral origin have inherent properties facilitating such preferential binding and/or have been modified to effect preferential targeting (see, e.g., Douglas et al., Nat. Biotech. 14:1574-1578, 1996; Kasahara, N. et al. Science 266:1373-1376, 1994; Miller, N., et al., FASEB Journal 9: 190-199, 1995; Chonn, A., et al., Curr. Opin. in Biotech. 6: 698-708, 1995; Schofield, J P, et al., British Med. Bull. 51: 56-71, 1995; Schreier, H, Pharmaceutica Acta Helvetiae 68: 145-159, 1994; Ledley, F. D., Hum. Gene Ther. 6: 1129-1144, 1995; Conary, J. T., et al., WO 95/34647 (21 Dec. 1995); Overell, R. W., et al., WO 95/28494 (26 Oct. 1995); and Truong, V. L. et al., WO 96/00295 (4 Jan. 1996)).

Targeted vectors include vectors (such as viruses, non-viral protein-based vectors and lipid-based vectors) in which delivery results in transgene expression that is relatively limited to particular host cells or host cell types. By way of illustration, angiogenic transgenes to be delivered according to the present invention can be operably linked to heterologous tissue-specific promoters thereby restricting expression to cells in that particular tissue.

For example, tissue-specific transcriptional control sequences derived from a gene encoding a cardiomyocyte-specific myosin light chain (MLC) or myosin heavy chain (MHC) promoter can be fused to a transgene such as an FGF gene within a vector such as the adenovirus constructs described above. Expression of the transgene can therefore be relatively restricted to cardiac myocytes. The efficacy of gene expression and degree of specificity provided by cardiomyocyte-specific MLC and MHC promoters with lacZ have been determined (using a recombinant adenovirus system such as that exemplified herein); and cardiac-specific expression has been reported (see, e.g., Lee et al., J. Biol Chem 267:15875-15885, 1992).

Since the MLC promoter can comprise as few as about 250 bp, it easily fits within even size-restricted delivery vectors such as the adenovirus-5 packaging system exemplified herein. The myosin heavy chain promoter, known to be a vigorous promoter of transcription, provides another alternative cardiac-specific promoter, comprising less than about 300 bp. While other promoters, such as the troponin-C promoter do not provide tissue specificity, they are small and highly efficacious.

Targeted Gene Expression

An unexpected finding of the present invention is that the recombinant adenovirus is taken up very efficiently in the first vascular bed that it encounters. Indeed, in the animal model of Example 4, the efficiency of the uptake of the virus in the heart after intracoronary injection, was 98%, i.e., 98% of the virus was removed in the first pass of the virus through the myocardial vascular bed. Furthermore, serum taken from the animals during the injection was incapable of growing viral plaques (Graham, Virology, 163:614-617, 1988) until diluted 200-fold, suggesting the presence of a serum factor (or binding protein) that inhibits viral propagation. These two factors (efficient first pass attachment of virus and the possibility of a serum binding protein) may act together to limit gene expression to the first vascular bed encountered by the virus.

To further evaluate the extent to which gene transfer was limited to the heart following intracoronary gene transfer, polymerase chain reaction (PCR) was used to see whether there was evidence for extracardiac presence of viral DNA two weeks after gene transfer in two treated animals (Example 4 below). Animals showed the presence of viral DNA in their hearts but not in their retinas, skeletal muscles, or livers. The sensitivity of the PCR is such that a single DNA sequence per 5,000,000 cells would be detectable. Therefore these data demonstrated that no viral DNA was present in extracardiac tissues two weeks after gene delivery. These results were further confirmed using other angiogenic proteins and derivatives as described below. These findings are extremely important because they confirm the concept of cardiac transgene targeting (i.e. providing expression of the transgene in the heart, but not elsewhere). The localized transgene delivery and expression provide the advantage of safety, further enhancing the use of the present methods in the treatment of patients.

Propagation and Purification of Adenovirus Vectors

Recombinant viral vectors, such as adenoviral vectors, can be plaque purified according to standard methods. By way of illustration, the resulting recombinant adenoviral viral vectors can be propagated in human 293 cells (which provide E1A and E1B functions in trans) to titers in the preferred range of about 10¹⁰-10¹² viral particles/ml. Propagation and purification techniques have been described for a variety of viral vectors that can be used in conjunction with the present invention. Adenoviral vectors are exemplified herein but other viral vectors such as AAV can also be employed. For adenovirus, cells can be infected at 80% confluence and harvested 48 hours later. After 3 freeze-thaw cycles of the infected cells, the cellular debris is pelleted by centrifugation and the virus purified by CsCl gradient ultracentrifugation (double CsCl gradient ultracentrifugation is preferred). Prior to in vivo injection, the viral stocks can be desalted (e.g., by gel filtration through Sepharose columns such as Sephadex G25). The desalted viral stock can also be filtered through a 0.3 micron filter if desired. We typically concentrate and purify the viral stock by double CsCl ultracentrifugation, followed by chromatography on Sephadex G25 equilibrated with phosphate buffered saline (PBS). The resulting viral stock typically has a final viral titer that is at least about 10¹⁰-10¹² viral particles/ml.

Preferably, the recombinant adenovirus is highly purified and is substantially free of wild-type (potentially replicative) virus. For these reasons, propagation and purification can be conducted to exclude contaminants and wild-type virus by, for example, identifying successful recombinant virus with PCR using appropriate primers, conducting two rounds of plaque purification, and double CsCl gradient ultracentrifugation.

Delivery of Vectors Carrying an Angiogenic Transgene

The means and compositions which are used to deliver the vectors carrying angiogenic protein transgenes depend on the particular vector employed as is well known in the art. Typically, however, a vector can be in the form of an injectable preparation containing a pharmaceutically acceptable carrier/diluent such as phosphate buffered saline, for example. Other pharmaceutical carriers, formulations and dosages are described below.

The presently preferred means of in vivo delivery for heart disease (especially for vector constructs that are not otherwise targeted for delivery and/or expression that is restricted to the myocardium or other target tissue), is by injection of the vector into a blood vessel or other conduit directly supplying the myocardium or tissue, preferably by injection into one or both coronary arteries or other tissue-specific arteries (or by a bolus injection into peripheral tissue). By way of illustration, for delivery to the myocardium, such injection is preferably achieved by catheter introduced substantially (typically at least about 1 cm) within the lumen of one or both coronary arteries or one or more saphenous veins or internal mammary artery grafts or other conduits delivering blood to the myocardium. Preferably the injection is made in both left and right coronary arteries to provide general distribution to all areas of the heart (e.g., LAD, LCx and Right). By injecting an adenoviral vector preparation in accordance herewith, optionally in combination with a vasoactive agent to enhance gene delivery as described herein, it is possible to perform effective adenovirus-mediated angiogenic gene transfer for the treatment of cardiovascular disease, for example clinical myocardial ischemia, or peripheral vascular disease without any undesirable effects.

The vectors are delivered in an amount sufficient for the transgene to be expressed and to provide a therapeutic benefit. For viral vectors (such as adenovirus), the final titer of the virus in the injectable preparation is preferably in the range of about 10⁷-10¹³ viral particles which allows for effective gene transfer. An adenovirus vector stock preferably free of wild-type virus can be injected deeply into the lumen of one or both coronary arteries (or grafts), preferably into both right and left coronary arteries (or grafts), and preferably in an amount of about 10⁹-10¹¹ viral particles as determined by optical densitometry. Preferably the vector is delivered in a single injection into each conduit (e.g. into each coronary artery).

To further augment the localized delivery of the gene therapy vector, and to enhance gene delivery efficiency, in accordance with the present invention, one can infuse a vasoactive agent, preferably histamine or a histamine agonist or a vascular endothelial growth factor (VEGF) protein or a nitric oxide donor (e.g. sodium nitroprusside), into the tissue to be treated, either coincidently with or, preferably, within several minutes before, introduction of the angiogenic gene therapy vector.

By injecting the vector composition directly into the lumen of the coronary artery by coronary catheters, it is possible to target the gene rather effectively, and to minimize loss of the recombinant vectors to the proximal aorta during injection. This type of injection enables local transfection of a desired number of cells, especially cardiac myocytes, in the affected myocardium with angiogenic protein- or peptide-encoding genes, thereby maximizing therapeutic efficacy of gene transfer, and minimizing undesirable angiogenesis at extracardiac sites. For delivery to diseased tissues supplied by peripheral vasculature, the vector can be introduced into one or more arteries supplying such tissue, or as a bolus injection into the tissue.

Vector constructs that are specifically targeted to the myocardium, such as vectors incorporating myocardial-specific binding or uptake components, and/or which incorporate angiogenic protein transgenes that are under the control of myocardial-specific transcriptional regulatory sequences (e.g., cardiomyocyte-specific promoters) can be used in place of or, preferably, in conjunction with such directed injection techniques as a means of further restricting expression to the myocardium, (e.g. the ventricular myocytes). For vectors that can elicit an immune response, it is preferable to inject the vector directly into a blood vessel supplying the myocardium as described above, although the additional techniques for restricting the extracardiac delivery or otherwise reducing the potential for an immune response can also be employed. Vectors targeted to tissues supplied by the peripheral vasculature, such as vectors targeted to skeletal muscle or promoters specifically expressed in skeletal muscle, can likewise be employed.

As described in detail below, it was demonstrated that using techniques of the present invention for in vivo delivery of a viral vector containing an angiogenic transgene, transgene expression did not occur in hepatocytes and viral RNA could not be found in the urine at any time after intracoronary injection. In addition, no evidence of extracardiac gene expression in the eye, liver, or skeletal muscle could be detected by PCR two weeks after intracoronary delivery of transgenes in this manner.

A variety of catheters and delivery routes can be used to achieve intracoronary delivery, as is known in the art (see, e.g., the references cited above, including: Topol, E J (ed.), The Textbook of Interventional Cardiology, 2nd Ed. (W.B. Saunders Co. 1994); Rutherford, R B, Vascular Surgery, 3rd Ed. (W.B. Saunders Co. 1989); Wyngaarden J B et al. (eds.), The Cecil Textbook of Medicine, 19th Ed. (W. B. Saunders, 1992); and Sabiston, D, The Textbook of Surgery, 14th Ed. (W.B. Saunders Co. 1991)). Direct intracoronary (or graft vessel) injection can be performed using standard percutaneous catheter based methods under fluoroscopic guidance. Any variety of coronary catheter, or a Stack perfusion catheter, for example, can be used in the present invention. For example, a variety of general purpose catheters, as well as modified catheters, suitable for use in the present invention are available from commercial suppliers such as Advanced Cardiovascular Systems (ACS), Target Therapeutics, Boston Scientific and Cordis. Also, where delivery to the myocardium is achieved by injection directly into a coronary artery (which is presently most preferred), a number of approaches can be used to introduce a catheter into the coronary artery, as is known in the art. By way of illustration, a catheter can be conveniently introduced into a femoral artery and threaded retrograde through the iliac artery and abdominal aorta and into a coronary artery. Alternatively, a catheter can be first introduced into a brachial or carotid artery and threaded retrograde to a coronary artery. The capillary bed of the myocardium can also be reached by retrograde perfusion, e.g., from a catheter placed in the coronary sinus. Such a catheter may also employ a proximal balloon to prevent or reduce anterograde flow as a means of facilitating retrograde perfusion. For delivery to tissues supplied by the peripheral vasculature, catheters can be introduced into arteries supplying such tissues (e.g., femoral arteries in the case of the leg) or may be introduced, by example, as a bolus injection or infusion into the affected tissue.

Various combinations of vectors comprising angiogenic genes and catheters or other in vivo delivery devices (e.g., other devices capable of introducing a pharmaceutical composition, generally in buffered solution, into a blood vessel or into muscle) can be incorporated into kits for use in accordance with the present invention. Such kits may also incorporate one or more vasoactive agents to enhance gene delivery, and may further include instructions describing their use in accordance with any of the methods described herein.

Animal Models

Important prerequisites for successful studies of cardiovascular gene therapy are (1) constitution of an animal model that is applicable to clinical cardiovascular disease that can provide useful data regarding mechanisms for increased blood flow and/or contractile function, and (2) accurate evaluation of the effects of gene transfer. From this point of view, none of the earlier techniques are satisfactory. Thus, we have made use of porcine models that fulfill these prerequisites. The pig is a particularly suitable model for studying heart diseases of humans because of its relevance to human physiology. The pig heart closely resembles the human heart in the following ways. The pig has a native coronary circulation very similar to that of humans, including the relative lack of native coronary collateral vessels. Secondly, the size of the pig heart, as a percentage of total body weight, is similar to that of the human heart. Additionally, the pig is a large animal model, therefore allowing more accurate extrapolation of various parameters such as effective vector dosages, toxicity, etc. In contrast, the hearts of animals such as dogs and members of the murine family have a lot of endogenous collateral vessels. Additionally, relative to total body weight, the size of the dog heart is twice that of the human heart.

An animal model described herein in Example 5 is exemplary of myocardial ischemia. (Since, myocardial ischemia can also result in and/or occur in connection with congestive heart failure, this particular model is further relevant to that situation.) Using this model, it was demonstrated that vector-mediated delivery of a gene encoding an angiogenic protein alleviated myocardial ischemia and enhanced blood flow in the ischemic region. Collateral vessel development was likewise increased. By way of illustration, we have successfully demonstrated these gene transfer techniques with several different angiogenic proteins, including both native forms and muteins (as described in detail in the Examples below).

In this model, which mimics clinical coronary artery disease, placement of an ameroid constrictor around the left circumflex (LCx) coronary artery results in gradually complete closure (within 7 days of placement) with minimal infarction (1% of the left ventricle, 4±1% of the LCx bed) (Roth, et al., Circulation 82:1778, 1990; Roth, et al., Am. J. Physiol., 235:1-11279, 1987; White, et al., Circ. Res., 71:1490, 1992; Hammond, et al., Cardiol., 23:475, 1994; and Hammond, et al., J. Clin. Invest., 92:2644, 1993). Myocardial function and blood flow are normal at rest in the region previously perfused by the occluded artery (referred to as the ischemic region), but blood flow reserve is insufficient to prevent ischemia when myocardial oxygen demands increase, due to limited endogenous collateral vessel development. Thus, the LCx bed is subject to episodic ischemia, analogous to clinical angina pectoris. Collateral vessel development and flow-function relationships are stable within 21 days of ameroid placement, and remain stable for four months (Roth, et al., Circulation, 82:1778, 1990; Roth, et al., Am. J. Physiol., 235:H1279, 1987; White, et al., Circ. Res., 71:1490, 1992). It has been shown by telemetry that animals have periodic ischemic dysfunction in the bed at risk, throughout the day, related to abrupt increases in heart rate during feeding, interruptions by personnel, etc. Thus, the model has a bed with stable but inadequate collateral vessels, and is subject to periodic ischemia. Another distinct advantage of the model is that there is a normally perfused and functioning region (the LAD bed) adjacent to an abnormally perfused and dysfunctioning region (the LCx bed), thereby offering a control bed within each animal.

Myocardial contrast echocardiography was used to estimate regional myocardial perfusion. The contrast material is composed of microaggregates of galactose and increases the echogenicity (whiteness) of the image. The microaggregates distribute into the coronary arteries and myocardial walls in a manner that is proportional to blood flow (Skyba, et al., Circulation 90:1513-1521, 1994). It has been shown that peak intensity of contrast is closely correlated with myocardial blood flow as measured by microspheres (Skyba, et al., Circulation, 90:1513-1521, 1994). To document that the echocardiographic images employed in the present invention were accurately identifying the LCx bed, and that myocardial contrast echocardiography could be used to evaluate myocardial blood flow, a hydraulic cuff occluder was placed around the proximal LCx adjacent to the ameroid.

In certain aspects of the present study, when animals were sacrificed, the hearts were perfusion-fixed (glutaraldehyde, physiological pressures, in situ) in order to quantitate capillary growth by microscopy. PCR was used to detect angiogenic protein DNA and mRNA in myocardium from animals that had received gene transfer. As described below, two weeks after gene transfer, myocardial samples from lacZ-transduced animals showed substantial beta-galactosidase activity on histological inspection. Finally, using a polyclonal antibody to an angiogenic protein, angiogenic protein expression in cells and myocardium from animals that had received gene transfer was demonstrated.

With respect to demonstrating improved blood flow, various techniques are known to those of skill in the art. For example, myocardial blood flow can be determined by the radioactive microsphere technique as described in Roth, D M, et al., Am. J. Physiol. 253:H1279-H1288, 1987 or Roth, D M, et al., Circulation 82:1778-1789, 1990. Myocardial blood flow can also be quantitated, e.g., by thallium imaging, which involves perfusing the heart with the radionuclide thallium as described by Braunwald in Heart Disease, 4^{th} ed., pp. 276-311 (Saunders, Philadelphia, 1992). The cells in the heart have an avidity for thallium. Uptake of thallium is positively correlated with blood flow. Thus, reduced uptake indicates reduced blood flow as occurs in ischemic conditions in which there is a perfusion deficit. In a conscious individual, angiogenic activity can be readily evaluated by contrast echocardiography such as described in Examples 1 and 5 and in Sahn, D J, et al., Circulation. 58:1072-1083, 1978. Improved myocardial function can be determined by measuring wall thickening such as by transthoracic echocardiography.

The strategy for therapeutic studies included the timing of transgene delivery, the means and route of administration of the transgene, and choice of the angiogenic gene. In the ameroid model of myocardial ischemia, gene transfer was performed after stable but insufficient collateral vessels had developed. Previous studies using the ameroid model had involved delivery of angiogenic peptides during the closure of the ameroid, prior to the development of ischemia and collateral vessels. However, that approach was not employed for several reasons. First, such studies are not suitable for closely duplicating the conditions that would be present in the treatment of clinical myocardial ischemia in which gene transfer would be given in the setting of ongoing myocardial ischemia; previous studies are analogous to providing the peptide in anticipation of ischemia, and are therefore less relevant. Second, it was presumed, based upon previous studies in cell culture, that an ischemic stimulus in conjunction with the angiogenic peptide would be the optimal milieu for the stimulation of angiogenesis. This could optimally be achieved by delivery of the transgene at a time when heart disease was already present. Linked to these decisions was the selection of the method to achieve transgene delivery. The constraint that the technique should be applicable for the subsequent treatment of patients with coronary disease made several approaches untenable (continuous infusion of a peptide into the coronary artery, direct plasmid injection into the heart, coating the heart with a resin containing the peptide to provide long-term slow release). Finally, the pig model provided an excellent means to follow regional blood flow and function before and after gene delivery. The use of control animals that received the same vector (e.g., a recombinant adenovirus), but with a reporter gene, provide a control for these studies. The pig has a native coronary circulation very similar of that of humans, including the relative lack of native coronary collateral vessels. The pig model also provided an excellent means to follow regional blood flow and function before and after gene delivery. The use of control animals that received the same recombinant adenovirus construct but with a reporter gene provided a control for these studies. Based on the foregoing, and previous published studies, those skilled in the art will appreciate that the results described below in pigs are expected to be predictive of results in humans.

With respect to peripheral vascular disease, delivery of angiogenic genes into the peripheral vasculature using gene therapy vectors of the present invention can be examined using, for example, a hind limb ligation model of peripheral ischemia. See, e.g., the femoral artery ligation model described by R. L. Terjung and colleagues (see, for example, Yang, et al., Circ. Res., 79(1)):62-9, 1996). As with delivery of angiogenic genes to ischemic myocardium, the delivery of angiogenic genes according to the present invention to the peripheral vasculature and/or associated muscle can be used to overcome effects of peripheral vascular disease.

Another animal model, described herein in Example 1, induces dilated cardiomyopathy such as that observed in clinical congestive heart failure. In this model, continuous rapid ventricular pacing over a period of 3 to 4 weeks induces heart failure which shows similarities with many features of clinical heart failure, including left ventricular dilation with impaired systolic function analogous to regional functional abnormalities seen in heart failure (including those associated with severe coronary artery disease and with non-CAD DCM, such as IDCM). Other animal models of congestive heart failure include the induction of chronic ventricular dysfunction via intracoronary delivery of microspheres (see e.g. Lavine et al., J Am Coll. Cardiol. 18: 1794-1803 (1991); Blaustein et al., Am. J. Cardio. Path. 5: 32-48 (1994); Sabbah et al. Am. J. Physiol. 260: H1379-H1384 (1991)). As an additional example of ventricular dysfunction, occlusion of the left coronary artery in a rat model can induce infarcts and the animals can then be studied and treated over subsequent days or weeks (see e.g. Pfeffer et al., Circ. Res. 44: 503-512, 1979; Pfeffer et al., Am. J. Physiol. 260:H1406-1414, 1991).

Thus, these models can be used to determine whether delivery of a vector construct coding for an angiogenic peptide or protein is effective to alleviate the cardiac dysfunctions associated with these conditions. These models are particularly useful in providing some of the parameters by which to assess the effectiveness of in vivo gene therapy for the treatment of congestive heart failure and ventricular remodeling.

Therapeutic Applications

The vectors of the present invention (such as the replication-deficient adenovirus) allow for highly efficient gene transfer in vivo without significant necrosis or inflammation. Based on these results, some of which are described in detail in the Examples below, it is seen that a sufficient degree of in vivo gene transfer to effect in vivo functional changes is achieved. The gene transfer of an angiogenic protein, either alone or in combination with another muscle enhancing protein or peptide, will improve blood flow and enhance muscle function in the treated muscle. Furthermore, if desired, a vasoactive agent can be employed in conjunction with these methods and compositions, as described herein, in order to further enhance gene delivery at the target site. Since a vasoactive agent, (such as histamine, a histamine agonist, a nitric oxide donor, or a VEGF protein) can be used to increase the efficiency of gene transfer at a gene vector dose, the inclusion of such an agent can be employed to limit the amount of vector required to be administered in order to achieve a given therapeutic effect.

In one aspect, the vectors and methods of the present invention can be employed to treat dilated cardiomyopathy (DCM), a type of heart failure that is typically diagnosed by the finding of a dilated, hypocontractile left and/or right ventricle. As discussed above, DCM can occur in the absence of other characteristic forms of cardiac disease such as coronary occlusion or a history of myocardial infarction. DCM is associated with poor ventricular function and symptoms of heart failure. In these patients, chamber dilation and wall thinning generally results in a high left ventricular wall tension. Many patients exhibit symptoms even under mild exertion or at rest, and are thus characterized as exhibiting severe, i.e. “Type-III” or “Type-IV”, heart failure, respectively (see, e.g., NYHA classification of heart failure). As noted above, many patients with coronary artery disease may progress to exhibiting dilated cardiomyopathy, often as a result of one or more heart attacks (myocardial infarctions).

A further application of the present invention is to prevent, or at least lessen deleterious left ventricular remodeling (a.k.a., deleterious remodeling, for short), which refers to chamber dilation after myocardial infarction that can progress to severe heart failure. Even if ventricular remodeling has already initiated, it is still desirable to promote an increase in blood flow, as this can still be effective to offset ventricular dysfunction. Similarly, promotion of angiogenesis can be useful, since the development of a microvascular bed can also be effective to offset ventricular dysfunction. Further, such angiogenic proteins or peptides can also have other enhancing effects. In a patient who has suffered a myocardial infarction, deleterious ventricular remodeling is prevented if the patient lacks chamber dilation and if symptoms of heart failure do not develop. Deleterious ventricular remodeling is alleviated if there is any observable or measurable reduction in an existing symptom of the heart failure. For example, the patient may show less breathlessness and improved exercise tolerance. Methods of assessing improvement in heart function and reduction of symptoms are essentially analogous to those described above for DCM. Prevention or alleviation of deleterious ventricular remodeling as a result of improved collateral blood flow and ventricular function and/or other mechanisms is expected to be achieved within weeks after in vivo angiogenic gene transfer in the patient using methods as described herein.

In one example, the present method of in vivo transfer of a transgene encoding an angiogenic protein is used to demonstrate that gene transfer of a recombinant adenovirus expressing an angiogenic protein or peptide is effective in substantially reducing myocardial ischemia. In another example, the present method of in vivo transfer of a transgene encoding an angiogenic protein is used to treat conditions associated with congestive heart failure.

As the data below shows, expression of an exogenously-provided angiogenic transgene results in increased blood flow and/or function in the heart (or other target tissue). This increased blood flow and/or function will lessen one or more symptoms of the cardiovascular disease affecting the target tissues.

As described herein, a number of different vectors can be employed to deliver the angiogenic protein transgenes in vivo according to the methods of the present invention. By way of illustration, the replication-deficient recombinant adenovirus vectors exemplified herein achieved highly efficient gene transfer in vivo without cytopathic effect or inflammation in the areas of gene expression.

In treating angina, as may be associated with CAD, gene transfer of an angiogenic protein encoding a transgene can be conducted at any time, but preferably is performed relatively soon after the onset of severe angina. In treating most congestive heart failure, gene transfer of an angiogenic protein encoding transgene can be conducted, for example, when development of heart failure is likely or heart failure has been diagnosed. For treating ventricular remodeling, gene transfer can be performed any time after the patient has suffered an infarct, preferably within 30 days and even more preferably within 7-20 days after an infarct.

As noted above, beta-adrenergic signaling proteins (beta-ASPs) (including beta-adrenergic receptors (beta-ARs), G-protein receptor kinase inhibitors (GRK inhibitors) and adenylylcyclases (ACs)) can also be employed to enhance cardiac function as described and illustrated in detail in U.S. patent application Ser. No. 08/924,757, filed 05 Sep. 1997 (based on U.S. 60/048,933 filed 16 Jun. 1997 and U.S. Ser. No. 08/708,661 filed 05 Sep. 1996), as well as PCT/US97/15610 filed 05 Sep. 1997, and U.S. continuing case Ser. No. 09/008,097, filed 16 Jan. 1998, and U.S. continuing case Ser. No. 09/472,667, filed 27 Dec. 1999, each of which is incorporated by reference herein.

Compositions or products of the invention may conveniently be provided in the form of formulations suitable for administration to a patient, into the blood stream (e.g. by intra-arterial injection or as a bolus infusion into tissue such as the skeletal muscle). A suitable administration format may best be determined by a medical practitioner. Suitable pharmaceutically acceptable carriers and their formulation are described in standard formulations treatises, e.g., Remington's Pharmaceuticals Sciences by E. W. Martin. See also Wang, Y. J. and Hanson, M. A., “Parental Formulations of Proteins and Peptides: Stability and Stabilizers”, Journals of Parental Sciences and Technology, Technical Report No. 10, Supp. 42:2S (1988). Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions also can be prepared to enhance shelf life and stability. The therapeutically useful compositions of the invention are prepared by mixing the ingredients following generally accepted procedures. For example, the selected components may be mixed to produce a concentrated mixture which may then be adjusted to the final concentration and viscosity by the addition of water and/or a buffer to control pH or an additional solute to control tonicity.

For use by the physician, the compositions will be provided in dosage form containing an amount of a vector of the invention which will be effective in one or multiple doses in order to provide a therapeutic effect. As will be recognized by those in the field, an effective amount of therapeutic agent will vary with many factors including the age and weight of the patient, the patient's physical condition, and the level of angiogenesis and/or other effect to be obtained, and other factors.

The effective dose of the viral vectors of this invention will typically be in the range of about 10⁷-10¹³ viral particles, preferably about 10⁹-10¹¹ viral particles. As noted, the exact dose to be administered is determined by the attending clinician, but is preferably in 5 ml or less of physiologically buffered solution (such as phosphate buffered saline), more preferably in 1-3 ml.

The preferred mode of administration is by injection into one or more localized sites (e.g., one or both coronary arteries, in the case of heart diseases) using a suitable catheter or other in vivo delivery device.

The following Examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### EXAMPLES

### Example 1

### Porcine Model of Congestive Heart Failure and Associated Myocardial Ischemia

1-A. Animals and Surgical Procedure

Nine Yorkshire pigs (Sus scrofa) weighing 40±6 kg were anesthetized with ketamine (50 mg/kg IM) and atropine sulfate (0.1 mg/kg IM) followed by sodium amytal (100 mg/kg IV). After endotracheal intubation, halothane (0.5% to 1.5%) was delivered by a pressure-cycled ventilator throughout the procedure. At left thoracotomy, catheters were placed in the aorta, pulmonary artery, and left atrium. A Konigsberg micromanometer was placed into the left ventricular apex, and an epicardial unipolar lead was placed 1.0 cm below the atrioventricular groove in the lateral wall of the left ventricle. The power generator (Spectrax 5985; Medtronic, Inc.) was inserted into a subcutaneous pocket in the abdomen. Four animals were instrumented with a flow probe (Transonic, Inc.) around the main pulmonary artery. The pericardium was loosely approximated and the chest closed. Seven to 10 days after thoracotomy, baseline measures of hemodynamics, left ventricular function, and myocardial blood flow were made. Ventricular pacing then was initiated (220±9 bpm (beats per minute) for 26±4 days). The stimulus amplitude was 2.5 V, the pulse duration 0.5 ms. Nine additional pigs (40±7 kg) were used as controls; five underwent thoracotomy and instrumentation without pacing and were killed 30±7 days after initial thoracotomy. Data regarding right and left ventricular mass were similar in the control animals whether they had undergone thoracotomy or not, so their data were pooled into a single control group.

1-B. Hemodynamic Studies

Hemodynamic data were obtained from conscious, unsedated animals after the pacemaker had been inactivated for at least 1 hour and animals were in a basal state. All data were obtained in each animal at 7-day intervals. Pressures were obtained from the left atrium, pulmonary artery, and aorta. Left ventricular dP/dt was obtained from the high-fidelity left ventricular pressure. Pulmonary artery flow was recorded. Aortic and pulmonary blood samples were obtained for calculation of arteriovenous oxygen content difference.

1-C. Echocardiographic Studies

Echocardiography is a method of measuring regional myocardial blood flow which involves injection of a contrast material into the individual or animal. Contrast material (microaggregates of galactose) increase the echogenicity (“whiteness”) of the image after left atrial injection. The microaggregates distribute into the coronary arteries and myocardial walls in a manner that is proportional to blood flow (Skyba, et al., Circulation. 90:1513-1521, 1994). The peak intensity of contrast enhancement is correlated with myocardial blood flow as measured by microspheres (Skyba, et al., Circulation, 90:1513-1521, 1994).

Two-dimensional and M-mode images were obtained with a Hewlett Packard Sonos 1500 imaging system. Images were obtained from a right parasternal approach at the mid-papillary muscle level and recorded on VHS tape. Measurements were made according to criteria of the American Society of Echocardiography (Sahn, D J, et al., Circulation. 58:1072-1083, 1978). Because of the midline orientation of the porcine interventricular septum (IVS) and use of the right parasternal view, short-axis M-mode measures were made through the IVS and the anatomic lateral wall. All parameters, including end-diastolic dimension (EDD), end-systolic dimension (ESD), and wall thickness, were measured on at least five random end-expiratory beats and averaged. End-diastolic dimension was obtained at the onset of the QRS complex. End-systolic dimension was taken at the instant of maximum lateral position of the IVS or at the end of the T wave. Left ventricular systolic function was assessed by use of fractional shortening, FS=[(EDD-ESD)/EDD]×100. Percent wall thickening (% WTh) was calculated as % WTh=[(ESWTh-EDWTh)/EDWTh]×100. To demonstrate reproducibility of echocardiographic measurements, animals were imaged on 2 consecutive days before the pacing protocol was initiated. The data from the separate determinations were highly reproducible (fractional shortening, R²=0.94, P=0.006; lateral wall thickening, R²=0.90, P=0.005). All of these measurements were obtained with pacemakers inactivated.

1-D. Myocardial Blood Flow

Myocardial blood flow was determined by the radioactive microsphere technique as described in detail in previously (Roth, D M, et al., Am. J. Physiol. 253:H1279-H1288, 1987; Roth, D M, et al., Circulation 82:1778-1789, 1990). Transmural samples from the left ventricular lateral wall and IVS were divided into endocardial, midwall, and epicardial thirds, and blood flow to each third and transmural flow were determined. Transmural sections were taken from regions in which echocardiographic measures had been made so that blood flow and functional measurements corresponded within each bed. Microspheres were injected in the control state (unpaced), at the initiation of ventricular pacing (225 bpm), and then at 7-day intervals during ventricular pacing at 225 bpm; microspheres were also injected with the pacemakers inactivated at 14 days (n=4) and 21 to 28 days (n=3). Myocardial blood flow per beat was calculated by dividing myocardial blood flow by the heart rate (recorded during microsphere injection) (Indolfi, C., et al., Circulation 80:933-993, 1989). Mean left atrial and mean arterial pressures were recorded during microsphere injection so that an estimate of coronary vascular resistance could be calculated; coronary vascular resistance index equals mean arterial pressure minus mean left atrial pressure divided by transmural coronary blood flow.

1-E. Systolic Wall Stress Circumferential systolic wall stress could not be determined because we could not obtain a suitable view to estimate the long axis of the left ventricle. Therefore, we calculated meridional end-systolic wall stress (Riechek, N., et al., Circulation 65:99-108, 1982) using the equation meridional end-systolic wall stress (dynes)=(0.334×P×D)÷[h(I−h/D)], where P is left ventricular end-systolic pressure in dynes, D is left ventricular end-systolic diameter in cm, and h is end-systolic wall thickness. Meridional end-systolic wall stress was calculated for both lateral wall and IVS before the initiation of pacing and subsequently at weekly intervals (pacemaker off).

1-F. Terminal Surgery

After 26±2 days of continuous pacing, animals were anesthetized and intubated, and midline sternotomies were made. The still-beating hearts were submerged in saline (4° C.), the coronary arteries were rapidly perfused with saline (4° C.), the right ventricle and left ventricle (including IVS) were weighed, and transmural samples from each region were rapidly frozen in liquid nitrogen and stored at a temperature of −70° C.

1-G. Adenine Nucleotides

ATP and ADP were measured in transmural samples of the IVS and lateral wall in four animals with heart failure (paced 28 days) and four control animals. The samples from the animals with heart failure were obtained with the pacemakers off (60 minutes) on the day the animals were killed. Samples were obtained identically in all animals. ATP and ADP were measured in a Waters high-performance liquid chromatograph as previously described (Pilz, R. B., et al., J. Biol. Chem. 259:2927-2935, 1984).

1-H. Statistical Analysis

Data are expressed as mean±standard deviation (SD). Specific measurements obtained in the control (prepaced) state and at 1-week intervals during pacing were compared by repeated measures ANOVA (Crunch4, Crunch Software Corp.). In some comparisons (lateral wall versus IVS, for example), two-way ANOVA was used. Post hoc comparisons were performed with the “Tukey method” as described in the art. Nine animals survived 21 days of pacing; six of these survived 28 days of pacing. Data from animals surviving 28 days were statistically indistinguishable from those who survived only 21 days. ANOVA was conducted, therefore, on nine animals at four time points: control (prepacing), 7 days, 14 days, and 21 to 28 days. The null hypothesis was rejected when P<0.05 (two-tailed).

### Results

1-I. Hemodynamic Studies

Rapid ventricular pacing resulted in changes in hemodynamics that were significant after 7 to 14 days of pacing. At 7 days, animals had increased mean left atrial and pulmonary arterial pressures. These pressures became increasingly abnormal with additional weeks of pacing (Table 1). Signs of circulatory congestion (tachypnea, ascites, and tachycardia) were evident by 14 to 21 days. Pulmonary arterial flow (cardiac output) had decreased by 21 days of pacing (control, 3.3±0.1 L/min; 21 days, 1.9±0.4 L/min; P<0.05).

TABLE 1
HEMODYNAMICS AND LEFT VENTRICULAR FUNCTION

| | n | Control | 7 d | 14 d | 21-28 d | p |
|---|---|---|---|---|---|---|
| HR (bpm) | 9 | 122 ± 16 | 136 ± 15 | 149 ± 13^{b} | 157 ± 15^{c,g} | .0004 |
| MAP (mm Hg) | 9 | 103 ± 8 | 99 ± 6 | 98 ± 7 | 102 ± 14 | .52 |
| PA (mm Hg) | 7 | 24 ± 7 | 37 ± 4^{b} | 42 ± 9^{c} | 48 ± 8^{c,g,i} | .0001 |
| LA (mm Hg) | 8 | 13 ± 3 | 25 ± 5^{c} | 30 ± 7^{c} | 36 ± 6^{c,e} | .0001 |
| AVO₂D(ml/dl) | 7 | 3.6 ± 1.1 | 3.5 ± 0.9 | 5.2 ± 1.5 | 6.2 ± 1.5^{b,h} | .0005 |
| EDD (cm) | 9 | 3.9 ± 0.4 | 4.4 ± 0.5 | 4.9 ± 0.6^{c} | 5.8 ± 0.6^{c,f,i} | .0001 |
| FS (%) | 9 | 39 ± 3 | 26 ± 5^{c} | 18 ± 6^{c,e} | 13 ± 4^{c,i} | .0001 |
| LV dP/dt (mm Hg/s) | 4 | 2849 ± 278 | 2408 ± 460 | 1847 ± 381^{c,d} | 1072 ± 123^{c,e,i} | .0001 |

Analysis of variance (repeated measures) was used to determine whether duration of pacing affected variable; p-values from ANOVA are listed in the rightward column.
Post hoc testing was performed by the Tukey method:
^{a}p < 0.05;
^{b}p < 0.01;
^{c}p < 0.001; (versus control value for the same variable);
^{d}p < 0.05;
^{e}p < 0.01;
^{f}p < 0.001 (vs. previous week);
^{g}p < 0.05;
^{h}p < 0.01;
ⁱp < 0.001 (vs. previous week);
post-hoc testing by Tukey method.
Measurements were made with pacemakers inactivited and represent mean ±SD.
7 d: 7 days of pacing;
14 d: 14 days of pacing;
21-28 d: 21-28 days of pacing.

1-J. Global Left Ventricular Function

Left ventricular function was assessed by echocardiography and hemodynamic variables after pacemakers had been inactivated. Fractional shortening was progressively reduced with duration of pacing (P=0.0001; Table 1), reaching its lowest value at 21 to 28 days (control, 39±3%; 21 to 28 days 13±4%; P<0.0002). Left ventricular end-diastolic dimension progressively increased during pacing (P<0.0001; Table 1), reaching its maximal value at 21 to 28 days (control, 3.9±0.4 cm; 21 to 28 days, 5.8±0.6 cm; P=0.0002). Left ventricular peak positive dP/dt also decreased throughout the study (P=0.0001; Table 1). The progressive fall in peak dP/dt was accompanied by increasing left ventricular end-diastolic pressure, documenting decreased left ventricular contractility, since increased preload normally augments left ventricular peak dP/dt. (Mahler, F., et al., Am. J. Cardiol. 35:626-634, 1975)

1-K. Left Ventricular Regional Function

With the pacemaker inactivated, regional left ventricular function was assessed by measurement of percent wall thickening of the left ventricular lateral wall and IVS. Ventricular pacing from the lateral wall caused significant deterioration in function of the lateral wall compared with the IVS (P=0.001; FIG. 1 and Table 2). This difference was significant at 7 days and increased further at 21 to 28 days as lateral wall function deteriorated. The IVS showed an insignificant decrease in wall thickening over the course of the study. End-diastolic wall thickness showed progressive thinning during the study that was more severe in the lateral wall (Table 2).

TABLE 2
SEQUENTIAL LEFT VENTRICULAR WALL THICKENING

| | CONTROL | 7 d | 14 d | 21-28 d | p(ANOVA) |
|---|---|---|---|---|---|
| IVS | .8 ± .1 | .7 ± .1 | .7 ± .1 | .6 ± .1 | time: .0001 |
| EDTh (cm) | | | | | |
| LAT | .8 ± .1 | .7 ± .1 | .6 ± .1 | .5 ± .1 | region: .039 |
| EDTh (cm) | | | | | |
| p (IVS | ns | ns | ns | ns | inter: .027 |
| vs. LAT) | | | | | |
| IVS | 33 ± 4 | 33 ± 5 | 28 ± 3 | 28 ± 6 | time: .0001 |
| WTh (%) | | | | | |
| LAT | 35 ± 5 | 25 ± 4 | 19 ± 8 | 14 ± 6 | region: .001 |
| WTh (%) | | | | | |
| p (IVS | ns | .02 | .007 | .0001 | inter: .0001 |
| vs. LAT) | | | | | |

Two-way analysis of variance (repeated measures) was used to determine whether end-diastolic wall thickness (EDTh) or % wall thickening (WTh) was affected by duration of pacing (time), or region (lateral wall, LAT; or interventricular septum, IVS), or whether the change in EDTh or WTh % was different between the two regions (inter). Mean values for EDTh and WTh % at each time point were tested for differences between the two regions post-hoc by Tukey analyses. Values represent mean±SD. 7 d: Seven days of pacing; 14 d: 14 days of pacing; 21-28 d: 21-28 days of pacing. n=9.

1-L. Left Ventricular Regional Blood Flow

Subendocardial blood flow per minute increased more in the IVS than in the lateral wall when pacing was initiated (FIG. 2 and Table 3). This difference in regional blood flow during pacing persisted for the duration of the study, and the pattern of change in blood flow was different between the two regions (P=0.006). The pattern of change in blood flow per minute between the two regions during pacing was consistent whether measured in endocardial (P=0.006), midwall (P=0.002), epicardial (P=0.016), or transmural (P=0.003) sections (Table 3). In contrast, when the pacemaker was inactivated, subendocardial blood flow showed no regional differences whether measured in the control state, at 14 days, or at 21 to 28 days (FIG. 2 and Table 3).

TABLE 3
SEQUENTIAL MYOCARDIAL BLOOD FLOW

| | DAY 0 | | DAY 14 | | DAY 21-28 | | |
|---|---|---|---|---|---|---|---|
| | OFF | ON | OFF | ON | OFF | ON | P(ANOVA) |
| IVS ENDO (ml/min/g) | 1.41 ± .26 | 1.96 ± .38 | 1.68 ± .22 | 2.35 ± .46 | 1.88 ± .18 | 2.67 ± .39 | time: .0001 |
| LAT ENDO (ml/min/g) | 1.40 ± .33 | 1.11 ± .14 | 1.50 ± .35 | 1.65 ± .25 | 1.73 ± .05 | 2.05 ± .16 | region: .017 |
| p (IVS vs. LAT) | Ns | .001 | ns | .002 | ns | .006 | inter: .006 |
| IVS MID (ml/min/g) | 1.56 ± .20 | 2.11 ± .33 | 1.84 ± .29 | 2.48 ± .31 | 2.04 ± .09 | 2.98 ± .46 | time: .0001 |
| LAT MID (ml/min/g) | 1.66 ± .28 | 1.53 ± .17 | 1.50 ± .43 | 1.77 ± .29 | 1.76 ± .39 | 2.12 ± .06 | region: .019 |
| p (IVS vs. LAT) | Ns | .01 | ns | ns | ns | .001 | inter: .002 |
| IVS EPI (ml/min/g) | 1.13 ± .27 | 1.50 ± .24 | 1.54 ± .38 | 1.91 ± .48 | 1.79 ± .14 | 2.53 ± .38 | time: .0001 |
| LAT EPI (ml/min/g) | 1.37 ± .22 | 1.48 ± .31 | 1.24 ± .24 | 1.55 ± .25 | 1.50 ± .04 | 1.92 ± .08 | region: .17 |
| p (IVS vs. LAT) | Ns | ns | ns | ns | .049 | ns | inter: .0016 |
| IVS TRANS (ml/min/g) | 1.36 ± .21 | 1.85 ± .27 | 1.69 ± .30 | 2.24 ± .46 | 1.90 ± .09 | 2.73 ± .40 | time: .0001 |
| LAT TRANS (ml/min/g) | 1.47 ± .27 | 1.38 ± .22 | 1.41 ± .33 | 1.65 ± .25 | 1.66 ± .02 | 2.03 ± .07 | region: .019 |
| p (IVS vs. LAT) | Ns | .001 | ns | ns | ns | .001 | inter: .003 |
| IVS ENDO/EPI | 1.30 ± .3 | 1.32 ± .23 | 1.13 ± .20 | 1.27 ± .26 | 1.05 ± .20 | 1.06 ± .10 | time: .058 |
| LAT ENDO/EPI | 1.01 ± .07 | 0.77 ± .10 | 1.21 ± .06 | 1.07 ± .11 | 1.15 ± .02 | 1.07 ± .10 | region: .054 |
| p (IVS vs. LAT) | Ns | .0002 | ns | ns | ns | ns | inter: .0008 |

Two-way analysis of variance (repeated measures) was used to determine whether subendocardial (ENDO) or transmural (TRANS) blood flow was affected by duration of pacing (time), or region (lateral wall, LAT; or interventricular septum, IVS), or whether the pattern of change in blood flow was different between the two regions (inter).
Mean values for blood flows at each time point were tested for differences between the two regions post-hoc by Tukey analyses.
Values represent mean ± SD from 5 animals.
ON: microspheres injected during ventricular pacing (225 bpm).
OFF: Pacemaker inactivated.
Day O = Control;
Day 14: 14 days of pacing;
Day 21-28: 21-28 days of pacing.

Endocardial-to-epicardial blood flow ratios did not change significantly as heart failure progressed (P=0.058). However, with the initiation of pacing, the endocardial-to-epicardial ratio was substantially lower in the lateral wall than in the IVS (IVS, 1.32±0.23; lateral wall, 0.77±0.10; P=0.0002; Table 3). Ratios in both regions were >1.0 throughout the rest of the study.

Endocardial blood flow per beat (FIG. 2 and Table 4) was similar in both regions before the initiation of pacing (IVS, 0.013±0.003 mL·min⁻¹·g⁻¹·beat⁻¹; lateral wall, 0.012±0.004mL·min⁻¹·g⁻¹·beat⁻¹; P=NS). On initiation of ventricular pacing (225 bpm), there was a regional deficit in endocardial blood flow per beat in the lateral wall but not in the IVS (IVS, 0.009±0.002 mL·min⁻¹g⁻¹beat⁻¹; lateral wall, 0.005±0.001 mL·min⁻¹·g⁻¹·beat⁻¹; P=0.001). At 14 days and 21 to 28 days, endocardial flow per beat was less in the lateral wall than in the IVS during pacing (FIG. 2 and Table 4). These data indicate that myocardial hypoperfusion in the lateral wall began with the onset of pacing, and this relative ischemia persisted. However, endocardial blood flows per beat remained normal in both regions with the pacemaker off (FIG. 2 and Table 4).

Blood flow in both regions tended to increase during the final week of pacing (FIG. 2 and Table 3). This pattern was associated with a progressive fall in the coronary vascular resistance index (FIG. 3), suggesting that alterations in coronary vascular structure and function may accompany left ventricular remodeling as heart failure progresses. The coronary vascular resistance index was significantly greater in the lateral wall than in the IVS at the initiation of pacing, and the pattern of change in coronary vascular resistance was different between the two regions (P=0.0012) (FIG. 3). These findings may indicate an effect of altered electrical activation on myocardial perfusion.

TABLE 4
ENDOCARDIAL BLOOD FLOW PER BEAT

| | |
|---|---|
| | ENDOCARDIAL FLOW PER BEAT |
| MODEL | (ml/mm/gram/beat) |

PORCINE AMAROID ISCHEMIA (HR 220 bpm; n = 6)

| | |
|---|---|
| NONISCHEMIC BED | 0.012 ± 0.004 |
| ISCHEMIC BED | 0.006 ± 0.002 |
| | p < 0.001* |

PORCINE LV PACING-INDUCED CHF (HR 225 bpm; n = 5)

| | | PACER ON | PACER OFF |
|---|---|---|---|
| DAY 0 | IVS | 0.009 ± 0.002 | 0.013 ± 0.003 |
| | | | (HR 122 bpm) |
| | LATERAL WALL | 0.005 ± 0.001 | 0.012 ± 0.004 |
| | | p = 0.001 | ns |
| DAY 14 | IVS | 0.010 ± 0.003 | 0.011 ± 0.002 |
| | | | (HR 149 bpm) |
| | LATERAL WALL | 0.007 ± 0.001 | 0.010 ± 0.003 |
| | | p = 0.008 | ns |
| DAY 21-28 | IVS | 0.012 ± 0.002 | 0.013 ± 0.003 |
| | | | (HR 157 bpm) |
| | LATERAL WALL | 0.009 ± 0.001 | 0.012 ± 0.002 |
| | | p < 0.024 | ns |

Data from ameroid ischemia model have been previously published from our laboratory (Hammond, H. K. and McKirnan, M. D., J. Am. Coll. Cardiol., 23: 475-82, 1994).
Values represent mean ± 1 SD.
These data show that the collateral-dependent (ischemic region) of the ameroid model and the lateral wall of the left ventricular pacing-induced heart failure model have similar deficits in endocardial blood flow per beat compared with myocardial regions that are normally perfused.

1-M. Left Ventricular End-Systolic Wall Stress

There was a significant increase in estimated meridional end-systolic wall stress with respect to duration of pacing (P<0.0001), but the pattern of change in wall stress was similar for the lateral wall and IVS (P=33), and post hoc testing failed to show any regional differences in systolic wall stress at any specific time point (FIG. 3). The increase in end-systolic wall stress was roughly threefold in the lateral wall (control, 168±40×10³ dynes; 28 days, 412±143×10³ dynes; P=0.0001) and in the IVS (control, 159±35×10³ dynes; 28 days, 480±225×10³ dynes; P=0.0001).

1-N. Necropsy

At necropsy, animals with heart failure had ascites (mean amount, 1809 mL; range, 300 to 3500 mL) and dilated, thin-walled hearts, with all four chambers appearing grossly enlarged. Ratios of ventricular weight to body weight suggested hypertrophy of the right ventricle only, confirming data from a previous study using this model. (Roth, D. A., et al., J. Clin. Invest 91:939-949, 1993) Compared with weight-matched control animals, there was no change in left ventricular mass associated with heart failure (control, 112±10 g; heart failure, 114±17 g); ratios of left ventricular weight to body weight were also similar in both groups (control, 2.8±0.3 g/kg; heart failure, 2.9±0.3 g/kg). In contrast, heart failure was associated with increased right ventricular weight (control, 38±3 g; heart failure, 52±11 g; P=0.003) and ratios of right ventricular weight to body weight (control, 0.09±0.1 g/kg; heart failure, 1.3±0.3 g/kg; P<0.003). Paced animals gained 4 kg during the course of the study, an amount accounted for in part by ascites accumulation. If the initial body weight is used to calculate the ratio of left ventricular weight to body weight, the ratio still is not significantly higher than that from weight-matched control animals. These data confirm that there was no substantive increase in left ventricular mass during the course of the study.

1-O. Adenine Nucleotides

Control animals showed normal ATP/ADP ratios, similar to those reported in pig heart collected by drill biopsies followed by immediate submersion in liquid nitrogen, (White, F. C., and Boss, G., J. Cardiovasc. Pathol. 3:225-236, 1990) documenting that the sampling techniques used were suitable. Animals with heart failure showed a marked reduction in ATP/ADP ratio in samples taken from the IVS (control, 14.8±1.1; heart failure, 2.4±0.3; P<0.0001, n=4 both groups) and from the lateral wall (control, 14.3±4.0; heart failure, 2.4±0.9; P=0.0012, n=4 both groups). This confirms an imbalance between myocardial oxygen supply and demand.

1-P. Myocardial Blood Flow

Regional variations in myocardial blood flow, an immediate consequence of rapid ventricular pacing, may play a role in the pathogenesis of regional and global dysfunction in pacing-induced heart failure. During pacing, a difference was found in myocardial blood flow per minute between the left ventricular lateral wall (adjacent to the site of stimulation) and the IVS. Reduced blood flow was present in the lateral wall immediately on the initiation of pacing and remained for 21 to 28 days. The left ventricular lateral wall, receiving less blood flow than the IVS during pacing, showed progressive reduction in wall thickening (pacer off) during 21 to 28 days of pacing. In contrast, the IVS, receiving greater blood flow during pacing, maintained relatively normal wall thickening through 21 to 28 days of pacing.

Since myocardial blood flow per minute does not readily permit assessment of relative myocardial ischemia, we also expressed coronary flow as endocardial blood flow per beat. The physiological basis for such an analysis lies in previous experiments showing that regional subendocardial blood flow per minute (rather than outer wall of transmural flow) is the primary determinant of regional myocardial contraction under conditions of progressive coronary artery stenosis (Gallagher, K. P., et al., Am. J. Physiol. 16:H727-H738, 1984) and that increases in heart rate shift this flow-function relation downward, with lower regional function at any level of subendocardial blood flow. (Delbaas, T., et al., J. Physiol. 477:481-496, 1990) However, if the flow-function relation is plotted as regional function versus endocardial blood flow per beat, to correct for heart rate effects, there is a single relation at different heart rates, indicating that endocardial blood flow per beat primarily determines the level of wall function when coronary blood flow is reduced. (Indolfi, C., et al., Circulation 80:933-993, 1989; Ross, J., Circulation 83:1076-1083, 1991) With the initiation of pacing, there was a >50% reduction in endocardial blood flow per beat in the lateral wall compared with the IVS (P<0.001; Table 4)

In prior studies in the conscious pig, we have documented that a 50% reduction in endocardial blood flow caused a 50% reduction of regional function and was associated with a subendocardial flow per beat similar to that observed in the lateral wall in the present studies (Table 4). The reduction in blood flow in the lateral wall during pacing persisted throughout the study. These data provide evidence for myocardial ischemia in the lateral wall on initiation of ventricular pacing. In contrast, IVS function and endocardial flow per beat remained relatively normal. With the pacemaker off, subendocardial blood flow per beat remained normal in both regions throughout the study, while regional dysfunction persisted in the lateral wall, consistent with the occurrence of myocardial stunning in that region. Thus, we postulate that sustained ischemia of the lateral wall has a significant effect on global function during and after pacing.

### Example 2

### Preparation of Illustrative Gene Delivery Constructs

2-A. Preparation of Illustrative Adenoviral Constructs

As an initial gene delivery vector, a helper independent replication deficient human adenovirus-5 system was used. As an initial illustration of vector constructs, we used the genes encoding β-galactosidase and FGF-5. Recombinant adenoviruses encoding β-galactosidase or FGF-5 were constructed using full length cDNAs. The system used to generate recombinant adenoviruses imposed a packing limit of about 5 kb for transgene inserts. Each of the β-gal and FGF-5 genes operably linked to the CMV promoter and with the SV40 polyadenylation sequences were less than 4 kb, well within the packaging constraints.

The full length cDNA for human FGF-5 was released from plasmid pLTR122E (Zhan et al., Mol. Cell. Biol., 8:3487, 1988) as a 1.1 kb ECOR1 fragment which includes 981 bp of the open reading frame of the gene and cloned into the polylinker of shuttle vector plasmid ACCMVpLpA. The nucleotide and amino acid sequence of FGF-5 is disclosed in FIG. 1 of Zhan et al., Mol. Cell. Biol., 8:3487, 1988. pACCMVpLpA is described in Gomez-Foix et al. J. Biol. Chem., 267:25129-25134, 1992. pACCMVpLpA contains the 5′ end of the adenovirus serotype 5 genome (map units 0 to 17) where the E1 region has been substituted with the human cytomegalovirus enhancer-promoter (CMV promoter) followed by the multiple cloning site (polylinker) from pUC 19 (plasmid well known in the art), followed by the SV40 polyadenylation signal. The lacZ-encoding control adenovirus is based on a E1A /E1B deletion from map unit 1 to 9.8. The FGF-5-encoding adenovirus (Ad.FGF-5) is based on a E1A/E1B deletion from map unit 1.3 to 9.3. Both of these vectors eliminate the entire E1A coding sequences and most of the E1B coding sequences. Both of the vectors have the transgene inserts cloned in an inverted orientation relative to the adenovirus sequences. Therefore, in the unlikely event of read-through transcription, the adenovirus transcript would be antisense and would not express viral proteins.

The FGF-5 gene-containing plasmid was co-transfected (using calcium phosphate precipitation) into 293 cells with plasmid JM17 (pJM17) which contains the entire human adenovirus 5 genome with an additional 4.3 kb insert, making pJM17 too large to be encapsidated into mature adenovirus virions. The cells were then overlaid with nutrient agarose. Infectious viral particles containing the angiogenic gene were generated by homologous rescue recombination in the 293 cells and were isolated as single plaques 10-12 days later. (Identification of successful recombinant virus also can be done by co-transfection by lipofection and directly looking for cytopathic effect microscopically as described in Zhang et al. Biotechniques 15(5):868-872, 1993). The resultant adenoviral vectors contain the transgene but are devoid of E1A/E1B sequences and are therefore replication-deficient. Adenovirus vector carrying the FGF-5 gene is also referred to herein as Ad.FGF-5.

Although these recombinant adenovirus were nonreplicative in mammalian cells, they could propagate in 293 cells which had been transformed with E1A/E1B and provided these essential gene products in trans. Recombinant virus from individual plaques was propagated in 293 cells and viral DNA was characterized by restriction analysis.

Successful recombinant virus then underwent two rounds of plaque purification using standard procedures. Viral stocks were propagated in 293 cells to titers in the range of 10¹⁰ to 10¹² per milliliter (ml) as determined by optical densitometry. Human 293 cells were infected at 80% confluence and culture supernatant was harvested at 36-48 hours. After subjecting the virus-containing supernatant to freeze-thaw cycles, the cellular debris was pelleted by standard centrifugation and the virus further purified by two cesium chloride (CsCl) gradient ultracentrifugations (discontinuous 1.33/1.45 CsCl gradient; CsCl prepared in 5 mM Tris, 1 mM EDTA (pH 7.8); 90,000×g (2 hr), 105,000×g (18 hr)). Prior to in vivo injection, the viral stocks were desalted by gel filtration through Sepharose columns (e.g. G25 Sephadex equilibrated with PBS). Final viral concentrations were about 10¹¹ viral particles per milliliter (ml), as determined by optical densitometry. Viral stocks can be conveniently stored in cells in media at minus 70 degrees C. For injections, purified virus is preferably resuspended in saline. The adenoviral vector preparation was highly purified and substantially free of wild-type (potentially replicative) virus (i.e., preferably containing less than about one (1) replication competent adenovirus (RCA) particle per million, more preferably less than 1 per 10⁹ and most preferably less than 1 per 10¹²). Thus, adenoviral infection and inflammatory infiltration in the heart were minimized.

Additional illustrations of adenoviral vector constructs are provided below and, in combination with the other teachings provided herein, other adenoviral vector constructs suitable for use in the present invention, including constructs based on modified adenoviral vectors, can be employed.

2-B. Additional Illustrative Vector and Transgene Constructs

As described above, various viral and non-viral vectors can be used to deliver genes in accordance with the present invention. As an illustrative example of another vector, adeno-associated viral (AAV) vectors have been generated for in vivo delivery according to the methods of the present invention as described above. As an illustrative example of another angiogenic gene that can be used in the context of the present invention, we have prepared constructs comprising an IGF gene as described above, in both adenoviral (Ad) and AAV vector constructs.

The exemplary constructs contain the IGF-1 gene under the control of a heterologous promoter (the CMV promoter was used for purposes of illustration), and are designated as rAd/IGF or rAAV/IGF. In addition to these, constructs comprising a marker gene, e.g., enhanced green fluorescent protein (EGFP), have been constructed. The rAd/IGF or rAAV/IGF constructs can also be constructed to include a marker gene (such as, EGFP). Constructs comprising EGFP are commercially available (for example, from Clontech, Palo Alto, Calif.).

To generate rAd/IGF, the IGF-1 gene (available from the ATCC) is subcloned into an adenovirus shuttle vector, such as pAdshuttle-CMV, pAd5CI, and/or pAdtrack-CMV. The resulting IGF-1 shuttle plasmid undergoes a recombination process with a helper plasmid, pJM17, in either bacteria or 293 cells depending on the shuttle vector used. The resulting rAd/IGF virus is verified for the expression of IGF-1 protein by RT-PCR and/or western blotting.

By way of illustration, we have prepared exemplary rAd/IGF constructs using the shuttle vector and pJM17 helper plasmid in 293 cells essentially as described and illustrated above for the generation of adenoviral vectors comprising the FGF-5 angiogenic gene. Adenoviral vectors comprising EGFP were prepared as controls using analogous techniques.

We have prepared exemplary rAAV/IGF constructs using techniques for the production of recombinant AAV vectors essentially as described in the art; see, e.g., the references related to AAV production as cited above. Although AAV vectors can be generated using a variety of different techniques, as described in the art, we used a basic double transfection procedure, essentially as described by Samulski et al., J. Virol. 63: 3822-3828, 1989. Briefly, to generate rAAV/IGF, the IGF-1 gene was subcloned into an rAAV plasmid DNA (such that the IGF gene would be flanked by the AAV inverted terminal repeats or ITRs) and this rAAV plasmid was then co-transfected into 293 cells with an AAV helper plasmid (to provide the AAV rep and cap genes in trans). AAV production was subsequently initiated by infecting with a helper adenovirus (we used an E1-deleted adenovirus known as dl312). Viral lysates are generally heat treated to inactivate adenovirus and treated with DNase and Pronase following standard techniques (see, e.g., Samulski et al., supra). rAAV/EGFP vectors were constructed using similar techniques.A variety of techniques can be employed for the purification of rAAV vectors. For purposes of this illustration, we used a standard cesium chloride (CsCl) ultracentrifugation procedure (using two CsCl purifications) for initially separating the rAAV particles from contaminants, essentially as has been described in the art. After dialysis, we further purified the material by HPLC. In this example, we used an affinity chromatography column which is coated with heparin (POROS HE, which is available from PE Biosystems, Foster City, Calif.), and eluted with salt (1 to 2M NaCl). In this example, most of the AAV eluted at approximately 0.7M NaCl. Following dialysis against PBS (pH 7.4), the vector was heat-treated at 56 degrees Celsius for 60 minutes to destroy residual adenoviruses. As with adenovirus, the resulting vector stocks are generally titered for DNase resistant particles (DRP); and are tested for absence of cytopathic effect.

Expression of IGF-1 in the rAd/IGF and rAAV/IGF was verified by western blot analysis. They were further tested for the production of functional IGF-1 protein using a proliferation assay on cultured MCF-7 cells. Briefly, HEK (human embryonic kidney carcinoma) 293 cells are transduced with rAd/IGF or rAAV/IGF on Day 1 and cultured in serum-free medium. After a 48 hour incubation, the serum free medium is harvested and put onto MCF-7 cells that have been cultured in serum-free medium. The proliferation of MCF-7 cells is monitored for the next 72 hours with a standard proliferation assay method (e.g. MTT assay), essentially as described by Mosmann (see e.g. Mosmann, J. Immunol. Meth. 16: 55-63, 1983). An adenovirus or AAV vector carrying enhanced green fluorescence protein gene, rAd/EGFP or rAAV/EGFP, was used as negative control and recombinant human IGF-1 protein was used as a positive control. Results from this MTT assay indicated that both the rAd/IGE and rAAV/IGF vector constructs were capable of delivering the IGF-1 transgene to the human target cells (HEK 293) and that the medium of such targeted cells was then capable of inducing proliferation of the MCF-7 cells in a manner analogous to purified IGF-1 protein. No significant proliferation was observed using medium from cells transfected with the negative controls (i.e. rAd/EGFP or rAAV/EGFP). We have also tested the vector constructs by directly transfecting MCF-7 cells and have demonstrated that the IGF constructs (in both AAV and adenovirus) can be used to directly induce proliferation in the transfected cells in a manner comparable to the administration of IGF-1 protein to the cells (at a concentration of about 3 micrograms/ml).

Additional tests to confirm the functionality of the IGF vector constructs can be performed using myocytes, in which the effects of IGF on muscle cell size and/or function can be observed. By way of illustration, the effect of IGF on primary neonatal cardiomyocytes (NCM), or adult cardiomyocytes, can be examined by various assays. For example, IGF-1 can be delivered by adenovirus or AAV vectors to induce hypertrophy and cellular DNA synthesis in NCM. After transduction of NCM at an appropriate multiplicity of infection (MOI), typically in the range of about 100 to 1000, the cardiomyocytes are stained with crystal violet or neutral red. The cells are imaged under a microscope, and the size of individual cells, including area, length, and width, can be measured automatically (e.g. using Image Plus software). The effect of IGF-1 on cellular DNA synthesis can be quantified by cellular incorporation of ³H-thymidine whereby the cellular DNA synthesis is monitored by ³H count after TCA precipitation of cellular DNA.

Vectors comprising angiogenic transgenes can be delivered to a heart by intracoronary delivery as described and illustrated herein. As an initial test of candidate vectors, prior to delivery in a large animal model such as pig, we have also employed a rat model in which we use indirect intracoronary delivery of vector to the myocardium. In that model, delivery is achieved by introduction of a solution comprising the vector (e.g. in phosphate buffered saline (PBS) or HEPES buffered saline) into the chamber of the left ventricle (i.e. by introduction into the lumen of the chamber as opposed to the ventricular wall) after constricting both the pulmonary artery and the distal aorta. Flow from the chamber of the ventricle thus carries the material to be delivered into the coronary arteries since alternative pathways are temporarily blocked. We have employed a cross-clamping procedure to constrict the pulmonary artery and aorta (see, e.g., Hajjar, et al., Proc. Natl. Acad. Sci. USA, 95: 5251-5256, 1998). We have also employed pretreatment with a vasoactive agent, as described herein and in the corresponding co-pending application referred to above, in order to enhance gene transfer via intracoronary delivery. By way of illustration, in administering vasoactive agent to pigs, as described herein, we typically use either histamine or sodium nitroprusside (SNP). These can be employed at ranges of about 1 to 75 micrograms/ml. Typically, we use about 25 micrograms/ml of histamine infused prior to delivery of vector. In the case of SNP, we typically use about 50 micrograms/ml of the vasoactive agent with infusion beginning up to several minutes prior to introduction of the vector and continuing until vector has been completely injected. In each case, the vasoactive agent was administered at a rate of about 1 ml/min, typically providing about 3 mL to each vessel receiving vector. Using these procedures, we have demonstrated very high levels of gene transfer to the myocardium via intracoronary delivery of both adenoviral and AAV vectors. Using rAAV/EGFP as described above, delivered at a dose of about 1×10e11 DNase-resistant particles, for example, we can achieve transduction of the left ventricle (LV) at levels of about 30% of cells (as measured by fluorescent microscopy, after fixing LV sections in paraformaldehyde and cutting with a cryostat into 8-10 micron sections, and quantifying the percentage of green area using ImagePro Plus software). Gene expression within the myocardium was greatest within the epicardium but significant expression was observed even in the endocardium. Additionally, we have demonstrated relatively long-lived gene expression (with little if any reduction in expression levels between 30 days and one year post-injection) following delivery of an AAV vector to the myocardium as described. Further, histological and pathological analyses revealed little or no inflammatory response in the heart and no detectable gene expression in either the liver or the lung.

### Example 3

### In Vitro and in Vivo Gene Transfer in Rats

3-A Ad.β-gal Gene Transfer and Expression

Adult rat cardiomyocytes were prepared by Langendorf perfusion with a collagenase containing perfusate according to standard methods. Rod shaped cells were cultured on laminin coated plates and at 24 hours, were infected with the β-galactosidase-encoding adenovirus obtained in the above Example 2, at a multiplicity of infection of 1:1. After a further 36 hour period, the cells were fixed with glutaraldehyde and incubated with X-gal. Consistently 70-90% of adult myocytes expressed the β-galactosidase transgene after infection with the recombinant adenovirus. At a multiplicity of infection of 1-2:1 there was no cytotoxicity observed.

3-B. rAAV/IGF-1 Gene Transfer and Expression

To assess the effects of IGF-1 expression in rat neonatal cardiac myocytes, 2×10e6 cells were plated on 10 cm cell culture dishes and infected with 1×10e10 DNase resistant particles of rAAV/IGF-1 or rAAV/EGFP. Cells were cultured without serum in minimal media and normal oxygen levels at 37 degrees Celsius. Recombinant IGF-1 protein (50 ng/ml) or phenylephrine (50 uM) were added to the culture as positive controls. Cells were visually assessed 48 hours after treatment. Cells treated with rAAV-IGF-1 displayed significant hypertrophy (comparable to that obtained using phenylephrine), based on morphological appearance, as compared to untreated cells. Exogenously-added IGF-1 protein appeared to induce only slight hypertrophy as compared to rAAV/IGF-1. To quantitate the level of hypertrophy, a stereological program, Image Pro Plus 5 (Media Cybernetics, Carlsbad, Calif.), was utilized. Briefly, the Image Pro Plus 5 program allows individual cells to be traced and measurements obtained. Cells were outlined within the program and area counts per cell were calculated. Approximately 50-100 cells were counted per condition and graphed in the statistical program Prizm. It was found that phenylephrine-treated cells and rAAV/IGF-1-infected cells demonstrated significant hypertrophy compared to untreated cells.

In addition to examining hypertrophy, the level of IGF-1 secretion into the media following rAAV/IGF-1 expression was determined using an ELISA assay for IGF-1 protein. Briefly, protein expression was found in the media of rAAV/IGF-1-infected cultures, collected at 48 hours, at levels of approximately 0.1-1.0 ng/ml, representing a significant increase over IGF-1 levels in control populations (untreated or infected with rAAV/EGFP).

3-C. In Vivo rAAV/EGFP Gene Transfer and Long Term Expression

Using EGFP-encoding AAV vectors in the rat model of indirect intracoronary delivery as described in Example 2, long-term gene expression was demonstrated. Briefly, following left thoracotomy, the aorta and pulmonary artery were cross-clamped and histamine (3 μg in 100 μL) or sodium nitroprusside (SNP) (2.5 μg in 100 μL) was delivered into the left ventricular chamber, through a vascular catheter, over about 10 seconds. The aorta and pulmonary artery were then unclamped for about 2 minutes to permit circulation of the vasoactive agent within the coronary vasculature. The arteries were reclamped and 2.5×10e11 particles of rAAV/EGFP, in about 340 μL buffer, were introduced into the left ventricular chamber over about 30 seconds. About 30 seconds later the arteries were unclamped, incisions sutured and animals permitted to recover.

Gene expression was measured 30 days (n=4), 60 days (n=4), 180 days (n=1) and 1 year (n=4) following gene delivery (accompanied by histamine) and measured 30 days (n=2) following gene delivery (accompanied by SNP). Substantial, sustained transmural gene transfer was detected in the left ventricle (LV), with an average of more than 20% of the LV transduced at each time point. Additionally, transgene expression was not detected in liver or lung samples.

### Example 4

### IN Vivo Gene Transfer Into Porcine Myocardium

4-A. Ad.β-gal Gene Transfer and Expression

The β-galactosidase-encoding adenoviral vector obtained in Example 2 was propagated in permissive 293 cells and purified by CsCl gradient ultracentrifugation with a final viral titer of 1.5×10¹⁰ viral particles, based on the procedures of Example 2. An anesthetized, ventilated 40 kg pig underwent thoracotomy. A 26 gauge butterfly needle was inserted into the mid left anterior descending (LAD) coronary artery and the vector (1.5×10¹⁰ viral particles) was injected in a 2 ml volume in phosphate buffered saline. The chest was closed and the animal allowed to recover. On the fourth post-injection day the animal was killed. The heart was fixed with glutaraldehyde, sectioned and incubated with X-gal for 16.5 hours. After imbedding and sectioning, the tissue was counterstained with eosin.

Microscopic analysis of tissue sections (transmural sections of LAD bed 96 hours after intracoronary injection of adenovirus containing lacZ) revealed a significant magnitude of gene transfer observed in the LAD coronary bed with many tissue sections demonstrating a substantial proportion of the cells staining positively for β-galactosidase. Areas of the myocardium remote from the LAD circulatory bed did not demonstrate X-gal staining and served as a negative control, while diffuse expression of a gene was observed in myocytes and in endothelial cells. A substantial proportion of myocytes showed β-galactosidase activity (blue stain), and, in subsequent studies using closed chest intracoronary injection, similar activity was present 14 days after gene transfer (n=8). There was no evidence of inflammation or necrosis in areas of gene expression.

4-B. rAAV/EGFP Gene Transfer and Expression

An EGFP-encoding adeno-associated viral vector was produced, propagated and purified as described above in Example 2. Four farm pigs (˜30 kg each) were anesthetized, ventilated and underwent a midline neck cutdown. The carotid artery was isolated and a 5 French Introducer sheath inserted. A 5 French multipurpose angiocatheter was placed in the left circumflex artery (LCX) with the tip of the catheter positioned about 1 cm within the coronary artery lumen. The syringe to be used for gene injection was first flushed with PBS and then the gene solution was drawn into the syringe. Intracoronary histamine, 25 μg/min, was infused for 3 min into the LCX prior to virus administration, followed by either 2.36×10¹³ viral particles of rAAV/EGFP (n=3) or 4.72×10¹³ viral particles of rAAV/EGFP (n=1). A total volume of 1.5 ml of gene solution was injected into each pig at an infusion rate of 1 ml/30 seconds. The angiocatheter and introducer sheath were then removed and the neck incision closed. The animals were allowed to recover from anesthesia and placed in their holding cage until completion of the study.

At 6-8 weeks post gene injection, pigs were sacrificed and tissues collected. Hearts were excised and placed in iced saline. Coronary arteries were cold perfused and the tissue collected and flash frozen in liquid nitrogen. Other tissues were likewise collected as quickly as possible and flash frozen in liquid nitrogen. Both fluorescence microscopy and RT-PCR of tissue sections demonstrated successful delivery and expression of the EGFP gene by rAAV vector using this direct intracoronary injection method of delivery in closed-chest pigs. In particular, as shown below, results from RT-PCR confirmed the gene was successfully delivered to and expressed in the bed supplied by the injected artery (i.e., the LCX bed) as compared to the left anterior descending coronary artery (LAD) bed:

| | Pig #1 | Pig #2 | Pig #3 | Pig #4 |
|---|---|---|---|---|
| LCX section 1 | + | + | + | + |
| LCX section 2 | + | − | + | + |
| LAD | − | − | − | − |

### Example 5

### Porcine Model of Angiogenesis-Mediated Gene Therapy (Using an FGF-5 Transgene)

In this pig model for myocardial ischemia and heart failure, animals were subjected to stress by atrial electrical stimulation (pacing). The degree of stress-induced myocardial dysfunction and inadequate regional blood flow was quantified and then gene transfer was performed by intracoronary injection of an illustrative recombinant adenovirus expressing FGF-5. Gene transfer was performed after stable but limited endogenous angiogenesis had developed, and inducible ischemia, analogous to angina pectoris in patients, was present. The animals had no ischemia at rest but developed ischemia during activity or atrial pacing. Control animals received a recombinant adenovirus expressing lacZ (β-gal) to exclude the possibility that the adenovirus itself, independent of FGF-5, was stimulating new blood vessel formation. This also controlled for possible continued collateral vessel development unrelated to gene transfer. Two weeks after gene transfer, stress-induced cardiac dysfunction and regional blood flow were again measured.

Pigs receiving lacZ showed a similar degree of pacing-induced dysfunction in the ischemic region before and two weeks after gene transfer. In contrast, two weeks after receiving the FGF-5 gene, the animals showed increase in wall thickening and improved blood flow in the ischemic region during pacing. The results demonstrated that gene transfer of an angiogenic transgene (FGF-5) was effective to ameliorate regional myocardial contractile dysfunction by improving regional blood flow through newly-formed blood vessels.

Methods

Animals and Model.

Yorkshire domestic pigs (Sus scrofa, n=27) weight 47±9 kg were used. Two animals underwent intracoronary injection of a recombinant adenovirus expressing lacZ (10¹¹ viral particles in 2.0 ml saline) and were killed 3 or 5 days after injection. The remaining 25 animals had catheters placed in the left atrium, pulmonary artery and aorta, providing a means to measure regional blood flow, and to monitor pressures. Wires were sutured on the left atrium to permit ECG recording and atrial pacing. An ameroid constrictor placed around the proximal left circumflex coronary artery. The ameroid material is hygroscopic and slowly swells, leading gradually to complete closure of the artery 10 days after placement, with minimal infarction (<1% of the left ventricle) because of the development of collateral blood vessels. Myocardial function and blood flow are normal at rest in the region previously perfused by the occluded artery (the ischemic region), but blood flow is insufficient to prevent ischemia when myocardial oxygen demands increase. Collateral vessel development is complete within 21 days of ameroid placement and remains unchanged for at least 4 months (Roth et al., Am. J. Physiol. 253: H1279-H1288, 1987). A hydraulic cuff was also placed around the artery, adjacent but distal to the ameroid. These procedures have been described in detail elsewhere (Hammond et al., J. Am. Coll. Cardiol. 23: 475-482, 1994 and Hammond et al., J. Clin. Invest. 92: 2644-2652, 1993). Two animals died 5 and 7 days after ameroid placement. Thirty-eight (±2) days after ameroid placement, when limited collateral circulation had developed and stabilized, animals underwent studies to define pacing-induced regional function and blood flow and then received recombinant adenovirus expressing either lacZ (n=7, control animals) or FGF-5 (n=16, treatment group) delivered by intracoronary injection. Then, 14±1 days later, studies to define pacing-induced regional function and blood flow were repeated. The following day, AdlacZ (n=7) and AdFGF-5 (n=11) animals were killed and tissues collected. Five AdFGF-5 animals were studied 12 weeks after gene transfer and then killed.

Recombinant Adenovirus and Transgene Delivery.

A helper-independent replication-deficient human adenovirus-5 system was prepared as described in Example 2 above.

For intracoronary delivery of the transgene, animals were anesthetized, and a 5F arterial sheath placed into the carotid artery. A 5F multipurpose (A2) coronary catheter was inserted through the sheath and into the coronary arteries. Closure of the ameroid was confirmed in all animals by contrast injection into the left main coronary artery. The catheter tip was then placed deeply within the arterial lumen so that minimal material would be lost to the proximal aorta during injection. Four milliliters containing 2×10¹¹ viral particles of recombinant adenovirus was delivered by slowly injecting 2.0 ml into both the left and right coronary arteries.

Assays:

(i) Regional Contractile Function and Perfusion.

Two-dimensional and M-mode images were obtained from a right parasternal approach at the papillary muscle level using a Hewlett Packard ultrasound imaging system (Hewlett-Packard Sonos 1000). Conscious animals were studied suspended in a comfortable sling to minimize body movement. Images were recorded on VHS tape with animals in a basal state and again during left atrial pacing (heart rate=200 beats per min). These studies were performed 1 day before gene transfer and repeated 14±1 days later. Five animals were examined again 12 weeks after gene transfer with FGF-5 to determine whether the effect on improved function was persistent. Rate-pressure products and left atrial pressures were similar in both groups before and after gene transfer, indicating similar myocardial oxygen demands and loading conditions. Echocardiographic measurements were made using standardized criteria (Sahn et al., Circulation 58: 1072-1083, 1978). To demonstrate reproducibility of echocardiographic measurements, animals (n=5) were imaged on two consecutive days. The data from the separate determinations were highly reproducible (lateral wall thickening: r²=0.90; P=0.005). The percent decrease in function measured by transthoracic echocardiography and sonomicrometry in this model are similar (Hammond et al., J., Am. Coll. Cardiol. 23: 475-482, 1994 and Hammond et al. J. Clin. Invest. 92: 2644-2652, 1993), documenting the accuracy of echocardiography for evaluation of ischemic dysfunction. Analysis was performed without knowledge of treatment group.

Contrast material (Levovist; microaggregates of galactose) increases the echogenicity (whiteness) of the image after left atrial injection. The microaggregates distribute into the coronary arteries and myocardial walls in a manner that is proportional to blood flow. The peak intensity of contrast enhancement is correlated with myocardial blood flow as measured by microspheres (Skyba et al., Circulation 58: 1072-1083, 1978). Thirty-eight (±2) days after ameroid placement, well after ameroid closure, but before gene transfer, contrast echocardiographic studies were performed during atrial pacing (200 bpm). Studies were repeated 14±1 days after gene transfer, and, in five animals, 12 weeks after gene transfer with FGF-5. Peak contrast intensity was measured from the video images with a computer-based video analysis program (Color Vue II, Nova Microsonics, Indianapolis, Ind.), that provided an objective measure of video intensity. Data were expressed as the ratio of the peak video intensity in the ischemic region (LCx bed) divided by the peak video intensity in the interventricular septum (IVS, a region receiving normal blood flow through the unoccluded left anterior descending coronary artery). The differences in regional blood flow during atrial pacing measured by contrast echocardiography were similar to the differences measured by microspheres in this same model in our laboratory, documenting the accuracy of echocardiography for the evaluation of regional myocardial blood flow. The contrast studies were analyzed without knowledge of which gene the animals had received.

(ii) Assessment of Angiogenesis.

The brachiocephalic artery was cannulated and other great vessels ligated. After intravenous injection of heparin (10,000 IU), papaverine (60 mg), and then potassium chloride (to induce diastolic cardiac arrest), the aorta was cross-clamped and the coronary vasculature perfused. Glutaraldehyde solution (6.25%, 0.1 M cacodylate buffer) was perfused at 120 mm Hg pressure; the heart was removed; the beds were identified using color-coded dyes injected anterograde through the left anterior descending, left circumflex and right coronary arteries; and the ameroid was examined to confirm closure. Samples taken from the normally perfused and ischemic regions (endocardial and epicardial thirds) were plastic-embedded and prepared for microscopic analysis of capillary number. Four 1-μm-thick transverse sections were taken from each subsample (endocardium and epicardium of each region) as previously described (Mathieu-Costello, Microvasc. Res. 33: 98-117, 1987 and Poole & Mathieu-Costello, Am. J. Physiol. 259: H204-H210, 1990). The number of capillaries around each fiber and fiber cross-sectional area in each of eight fields in each subsample (randomly selected by systematic sampling) were measured with an image analyzer (Videometric 150, American Innovision) at X1400. The number of capillaries around a total of 325±18 fibers was measured. Capillary density (number per fiber cross-sectional area) was estimated by point counting 15±1 fields per subsample. The relative standard errors of capillary number around a fiber, fiber cross-sectional area and capillary density were 1.4, 4.1 and 4.2% respectively. Capillary-to-fiber ratio was calculated as the product of capillary density and fiber cross-sectional area. There was no significant difference in fiber cross-sectional area in myocardial samples from either group. Bromodeoxyuridine (50 mg/kg) was injected into the peritoneal space of five animals: a control animal (no ameroid); two animals with ameroid occluders that received lacZ gene transfer 2 weeks before; and two animals with ameroid occluders that received FGF-5 gene transfer 2 weeks before. Thirty-six hours after BRDU injection the animals were killed, and the tissue was prepared for analysis using methods previously described (Kajstura et al., Circ. Res. 74: 383-400, 1994). Sections of duodenum were used as positive controls.

(iii) DNA, mRNA and Protein Expression.

Following gene transfer, left ventricular homogenates underwent studies to document transgene presence and expression. The polymerase chain reaction (PCR), using a sense primer to the CMV promoter (GCAGAGCTCGTTTAGTGAAC; SEQ ID. NO. 1) and an antisense to the internal FGF-5 sequence (GAAAATGGGTAGAGATATGCT; SEQ ID NO. 2) amplified the expected 500-bp fragment. Using a sense primer to the beginning of the FGF-5 sequence (ATGAGCTTGTCCTTCCTCCTC; SEQ ID NO. 3) and an antisense primer to the internal FGF-5 sequence (i.e., SEQ ID NO. 2), RT-PCR amplified the expected 400-bp fragment. Primers directed against the adenovirus DNA E2 region were used to detect wild-type or recombinant viral DNA in tissues (TCGTTTCTCAGCAGCTGTTG; SEQ ID NO. 4) and (CATCTGAACTCAAAGCGTGG; SEQ ID NO. 5). The expected 900-bp fragment was amplified from the recombinant virus. These studies were conducted on 200-mg tissue samples from myocardium and other tissues. PCR detection sensitivity was 1 viral sequence per 5 million cells. A polyclonal antibody directed against FGF-5 (Kitaoka et al., Invest. Ophthalmol. Vis. Sci. 35:3189-3198, 1994) was used in immunoblots of protein from the medium of cultured rat cardiac fibroblasts 48 h after the gene transfer of FGF-5 or lacZ. FGF-5 protein was found in conditioned media after gene transfer of FGF-5, but not after gene transfer of lacZ. Methods for PCR and western blotting have been described in detail elsewhere (Hammond et al., J. Clin. Invest. 92: 2644-2652, 1993, Roth et al. J. Clin. Invest. 91: 939-949, 1993, and Tsai et al. Am. J. Physiol. 267:H2079-H2085, 1994). To examine the transgene for mitogenic activity in vitro, adult rat cardiac fibroblasts were infected with adenovirus-encoding FGF-5 or with adenovirus-encoding lacZ, or were not infected. Media from these cell cultures were incubated with NIH 3T3 mouse fibroblasts, and tritiated thymidine incorporation was measured (Tsai et al., Endocrinology 136: 3831-3838, 1995).

(iv) Adenovirus Release During Intracoronary Delivery.

Pulmonary arterial blood was withdrawn continuously during intracoronary injection of recombinant adenovirus in three animals. Serum from each sample was used in a standard plaque assay. Undiluted serum (0.5 ml) was added to subconfluent H293 cells; 10 days later, no plaques had formed. However, when 0.5 ml serum was diluted 200- to 8000-fold with DMEM (2% FBS), viral plaques formed by day 9. A single vascular bed (myocardial) separates the coronary and pulmonary arteries. If no virus attaches in this bed after injection into the coronary artery, then the pulmonary artery concentration of virus should reflect the dilution of coronary sinus blood by systemic venous blood over the time of the injection. Measurements from our laboratory indicate that coronary flow represents 5% of pulmonary artery flow. Using this dilution factor (20-fold), the duration of coronary injection, and the amount of adenovirus injected, we calculated the amount of adenovirus delivered to the pulmonary artery, assuming no adenovirus escape or attachment. This estimate was compared to the measured amount and the difference used as an estimate of the amount of virus cleared by the myocardial vascular bed.

(v) Assessment of Inflammation.

Hematoxylin/eosin and Masson's trichrome stains were used to detect inflammatory cell infiltrates, cell necrosis and fibrosis. Mouse ascites, porcine anti-CD4 and anti-CD8 monoclonal antibodies (1.0 mg/ml; VMRD, Inc., Pullman, Washington) were used to detect CD4 and CD8 markers on T lymphocytes in frozen sections (6 μm) of spleen (positive control) and heart. These studies were performed on transmural samples of hearts of six animals that had received ameroid occluders 50 days before being killed: two animals received no gene transfer, two received FGF-5 gene transfer 2 weeks before, and two received lacZ gene transfer 2 weeks before. Analysis was conducted without knowledge of treatment group.

(vi) Statistical Analysis.

Data are expressed as means±1 s.e.m. Measurements made before and after gene transfer with FGF-5 and lacZ were compared using two-way analysis of variance (Crunch4, Crunch Software Corporation, Oakland, Calif.). Data from angiogenesis studies also underwent two-way analysis of variance. The null hypothesis was rejected when P<0.05.

Results Using an FGF-5 Transgene

Three measurements were used to assess whether gene transfer of FGF-5 was effective in treating myocardial ischemia: regional contractile function and perfusion (assessed before and after gene transfer) and capillary number. All measurements were conducted without knowledge of which gene the animals had received (FGF-5 versus lacZ).

Regional contractile function and blood flow. Thirty-eight days after ameroid placement, animals showed impaired wall thickening during atrial electrical stimulation (pacing). Pigs receiving lacZ showed a similar degree of pacing-induced dysfunction in the ischemic region before and two weeks after gene transfer. In contrast, two weeks after FGF-5 gene transfer there was a 2.7-fold increase in wall thickening in the ischemic region during pacing (P<0.0001; FIG. 6). Wall thickening in the normally perfused region (the interventricular septum) was normal during pacing and unaffected by gene transfer (% wall thickening: lacZ: pre gene transfer 53±8%, post gene transfer 51±6%; FGF-5: pre gene transfer 59±4%, post gene transfer 59±6%).

Associated with improved function in the ischemic region was improved regional blood flow. Two weeks after lacZ gene transfer there was a persistent flow deficit in the ischemic region during pacing (FIG. 8). Animals receiving FGF-5 gene transfer, however, showed homogeneous contrast enhancement in the two regions two weeks later, indicating improved blood flow in the ischemic region (P=0.0001). To determine whether improved function and perfusion in the ischemic bed were long lasting, five animals were examined again 12 weeks after FGF-5 gene transfer. Each animal showed persistent improvements in function (P=0.005; FIG. 6) and perfusion (P=0.001; FIG. 8).

Angiogenesis. Uninfected ameroid-constricted animals (no gene transfer performed) had identical physiological responses to animals receiving lacZ-encoding adenovirus, indicating that the lacZ vector did not adversely affect native angiogenesis. To assess angiogenesis, myocardial capillary number was quantified using microscopic analysis of perfusion-fixed hearts (FIG. 9). The number of capillaries surrounding each myocardial fiber was greater in the endocardium of the ischemic and nonischemic regions in animals that received gene transfer with FGF-5 when compared with the same regions of the hearts of animals that had received gene transfer with lacZ (P=0.038). Thus, improved regional function and perfusion were associated with capillary angiogenesis two weeks after FGF-5 gene transfer. Increased capillary number around each fiber tended to increase in the epicardial portion of the wall after FGF-5 gene transfer (P=0.13). Other measures of capillarity such as capillary number per fiber cross-sectional area and capillary number per fiber number were not changed in endocardium or epicardium.

DNA, mRNA and protein expression. Having established favorable effects of FGF-5 gene transfer on function, perfusion and capillary number around each fiber, it was imperative to demonstrate presence and expression of the transgene in the heart. Polymerase chain reaction (PCR) and reverse transcriptase coupled with PCR (RT-PCR).were used to detect transgenic FGF-5 DNA and mRNA in myocardium from animals that had received FGF-5 gene transfer.

Following gene transfer, left ventricular samples were examined to document transgene incorporation and expression. Briefly, 3 days after intracoronary gene transfer of lacZ, myocardium was treated with X-gal, and then counterstained with Eosin X120. Examination using standard histological techniques revealed that the majority of myocytes showed β-galactosidase activity (blue stain). Activity was also seen 14±1 days after gene transfer in all animals that had received lacZ gene transfer. Higher magnification demonstrated cross striations in cells containing β-galactosidase activity, confirming gene expression in myocytes. PCR analysis using a sense primer directed against the CMV promoter and an antisense primer directed against an internal FGF-5 sequence, was performed to confirm the presence of recombinant adenovirus DNA encoding FGF-5 in the ischemic (LCx) and nonischemic (LAD) regions of three animals that received FGF-5 gene transfer. The results, shown in FIG. 10A confirmed the presence of the expected 500-bp fragments. FGF-5 mRNA expression was then examined 14 days after gene transfer. As shown in FIG. 10B, the RT-PCR-amplified 400-bp fragment was present in both regions from two animals, whereas control animals showed no signal. A polyclonal antibody directed against FGF-5 was used in immunoblots of protein from the medium of cultured rat cardiac fibroblasts 48 hours after gene transfer of FGF-5 or lacZ. As shown in FIG. 10C, FGF-5 protein was found after gene transfer of FGF-5 (F), but not after gene transfer of lacZ (β), demonstrating protein expression and extracellular secretion after FGF-5 gene transfer. Finally, PCR, using a set of primers directed against adenovirus DNA (E2 region), was performed to determine whether adenovirus DNA was present in retina, liver, or skeletal muscle of two animals that received intracoronary injection of adenovirus 14 days before. As shown in FIG. 10D, the expected 900-bp amplified fragment was only found in a control lane (+) containing recombinant adenovirus (as a positive control), and not in the lanes derived from the retina (r), liver (l), or skeletal muscle (m) of the treated animals.

Successful gene transfer was documented in both the ischemic and nonischemic regions. Immunoblotting showed FGF-5 protein in myocardium from animals that received FGF-5 gene transfer. In additional experiments using cultured fibroblasts, we documented that gene transfer of FGF-5 conferred the ability of these cells to synthesize and secrete FGF-5 extracellularly. Media from cultured cells infected with recombinant adenovirus expressing FGF-5 showed a mitogenic response (14-fold increase versus control; P=0.005). Finally, two weeks after gene transfer, myocardial samples (but not liver samples) from lacZ-infected animals showed P-galactosidase activity on histological inspection. These studies confirm successful in vivo gene transfer and expression, and demonstrate the biological activity of the transgene product.

Two weeks after intracoronary injection of recombinant adenovirus, we were unable to detect viral DNA in liver, retina or skeletal muscle using PCR despite the presence of viral DNA in myocardium. Furthermore, viral DNA was undetectable in urine 2-24 hours after intracoronary injection. These experiments indicated that intracoronary delivery of the adenoviral vector minimized systemic arterial distribution of the virus to a level below the detection limits of the PCR methods. This technique might be difficult to achieve in animals with smaller coronary artery size such as rabbits.

To assess the efficiency of myocardial uptake of adenovirus, we measured the amount of adenovirus released from the heart by sampling pulmonary arterial blood during intracoronary injection. A surprising 98.7% of the virus was cleared by the heart on the first pass. Undiluted serum obtained from the pulmonary artery during intracoronary delivery of virus was incapable of forming viral plaques in appropriate conditions. Thus the present invention effectively provides a cardiac-specific gene delivery system.

Assessment of inflammation. Microscopic inspection of transmural sections of hearts of animals that had received recombinant adenovirus did not show inflammatory cell infiltrates, cell necrosis or increased fibrosis. As an additional evaluation for an adenovirus-induced cytopathic effect, we conducted immuno-histological studies to detect CD4 and CD8 antigens that would indicate the presence of cytotoxic T cells. These studies showed rare positive cells on transmural sections of heart from uninfected animals (n=2) or animals that had received recombinant adenovirus (n=4). The liver also was free from inflammation.

### Example 6

### Gene-Mediated Angiogenesis Using an FGF-4 Transgene

This experimental example demonstrated successful gene therapy using a different angiogenic protein-encoding gene, FGF-4. The protocol for FGF-4 gene therapy was essentially as described in Example 5 above for FGF-5.

The human FGF-4 gene was isolated from a cDNA library which was constructed from mRNA of Kaposi's Sarcoma DNA transformed-NIH3T3 cells. The FGF-4 cDNA is about 1.2 kb in length and encodes a polypeptide of 206 amino acids including a 30 amino acid signal peptide at the N-terminal (Dell Bovi et al. Cell 50:729-737, 1987; Bellosta et al. J. Cell Biol. 121:705-713, 1993). We subcloned the FGF-4 cDNA as an essentially full-length 1.2 kb EcoR1 fragment, into the EcoR1 site in adenovirus vector pACCMVpLpASR (pACSR for simplicity). The 5′ start site was at 243 basepairs and the 3′ end at 863 basepairs. Recombinant adenovirus encoding FGF-4 (also referred to herein as Ad.FGF-4) was made as described in Example 2 for making the FGF-5 adenovirus.

Expression of FGF-4 in cardiac tissue (and a lack of expression in other tissues including the liver, skeletal muscle and eye) was confirmed by Western-blot analysis using anti-FGF-4 antibody for detection. The mitogenic effect of FGF-4 on proliferation of endothelial cells in vitro was also tested.

Forty-five days after ameroid placement, animals underwent studies to define stress-induced regional function and blood flow and then received recombinant adenovirus expressing FGF-4 (n=6 animals) delivered by intracoronary injection. Thirteen days later, studies to define stress-induced regional function and blood flow were repeated. The following day, animals were killed and tissues collected.

Transgene Delivery

As with FGF-5, gene transfer was performed after endogenous angiogenesis was quiescent and inducible myocardial ischemia, analogous to angina pectoris in patients, was present. For intracoronary delivery of the transgene, animals were anesthetized, and a 5F arterial sheath placed into the carotid artery. A 5F multipurpose coronary catheter was inserted through the sheath and into the coronary arteries. Closure of the ameroid was confirmed in all animals by contrast injection into the left main coronary artery. The catheter tip was then placed 1 cm within the arterial lumen so that minimal material would be lost to the proximal aorta during injection. Five ml containing 1.5×10¹² viral particles of recombinant adenovirus expressing FGF-4 were delivered by slowly injecting 3.0 ml into the left and 2.0 ml into the right coronary arteries.

Results Using an FGF-4 Transgene

Regional Function and Perfusion

Forty-five days after ameroid placement, animals showed impaired wall thickening during atrial pacing. In contrast, two weeks after FGF-4 gene transfer there was a 2.7 fold increase in wall thickening in the ischemic region during pacing (p<0.0001; FIGS. 11 and 12). Wall thickening in the normally perfused region (the interventricular septum) was normal during pacing and unaffected by gene transfer. The improvement in function after FGF-4 gene transfer was statistically indistinguishable from the improvement obtained following gene transfer with FGF-5 or FGF-2LI+signal peptide (“sp”(FIG. 11). Improved function in the ischemic region was associated with improved regional perfusion (FIG. 12). As shown in FIG. 12, prior to FGF-4 gene transfer there was a flow deficit in the ischemic region during pacing. Two weeks after gene transfer with FGF-4, homogeneous contrast enhancement was seen in the two regions, indicating improved flow in the ischemic region (p=0.0001). Results with FGF-4 were statistically indistinguishable from results obtained with FGF-5 and FGF-2LI+sp (FIG. 13). FGF-2LI+sp is described in Example 7 and refers to FGF-2 containing a signal sequence.

Transgene Expression

Exclusive expression of FGF-4 in the heart was confirmed by performing PCR and RT-PCR using primers specific for sequences encoding the transgene. FGF-4 DNA and mRNA were found in the heart, but absent in the eye, liver, and skeletal muscle. These data confirm data derived from the use of Ad.FGF-5 (n=2) and Ad.FGF-2LI+sp (n=1). Thus exclusive expression of the transgene in the heart was confirmed in all four animals which had received adenoviral vectors containing different angiogenic protein-encoding genes.

Absence of Myocardial Inflammation

Transmural myocardial biopsies from three consecutive animals that received Ad.FGF-4 have been examined. The animals were killed 2 weeks after gene transfer. There was no evidence of inflammatory cell infiltrates, necrosis, or increased fibrosis in these sections compared to control ameroid animals that received no adenovirus. This was true in both the LAD and LCx beds. These slides were reviewed by a pathologist who made a blind-sample assessment and commented that there was no evidence for myocarditis in any section.

### Example 7

### Gene-Mediated Angiogenesis Using an FGF-2 Mutein

This experimental example demonstrated successful gene therapy using a third angiogenic protein-encoding gene, FGF-2. This experiment also demonstrates how an angiogenic protein can be modified to increase secretion and potentially improve efficacy of angiogenic gene therapy in enhancing blood flow and cardiac function within the heart. The protocol used for human FGF-2 gene therapy was virtually identical to that employed for FGF-5 and FGF-4 above.

Acidic FGF (aFGF, FGF-1) and basic FGF (FGF-2) lack a native secretory signal sequence; although some protein secretion may occur. An alternate secretary pathway, not involving the Golgi apparatus, has been described for acidic FGF. Two FGF-2 constructs (FGF-2LI+sp and FGF-2LI−sp) were made, one with a sequence encoding a signal peptide (FGF-2LI+sp) for the classic protein secretary pathway and one without the signal peptide encoding sequence (FGF-2LI−sp) to test for improved efficacy of FGF-2 having an added signal peptide over the same protein without the added signal peptide.

As shown below, FGF-2 has a five-residue loop structure which extends from amino acid residue 118 to residue 122. This loop structure was replaced by cassette directed mutagenesis, with the corresponding five-residue loop from interleukin-1βto produce FGF-2LI loop replacement mutants. Briefly, the gene encoding human Glu^{3,5}FGF-2 (Seddon et al. Ann. N.Y. Acad. Sci. 638:98-108, 1991) was cloned into T7 expression vector pET-3a (M13), a derivative of pET-3a (Rosenberg et al. Gene 56:125-135, 1987), between restriction sites Ndel and BamH1. The unique restriction endonuclease sites, BstB1 and Spl1, were introduced into the gene in such a way as to produce no change in the encoded amino acids (i.e. silent mutations) at positions that flank the codons encoding the segment Ser117-Trp123 of FGF-2.

Structured alignment¹ of the β9-β10 loops in FGF-1, FGF-2, and IL-1β.
¹ Numbering for FGF-1 and FGF-2 is from amino acid residue 1 deduced from the cDNA sequence encoding the 155-residue form (as described in Seddon et al. Ann. N.Y Acad. Sci. 638:98-108, 1991), and that for IL-1β is from residue 1 of the mature 153-residue polypeptide (id.).

| | |
|---|---|
| 110 115 120 125 130 | |
| FGF-1 ENHYNTYISKKHAEKHWFVGLKKNG | (SEQ ID NO.6) |
| 110 115 120 125 130 | |
| FGF-2 SNNYNTYRSRKY..TSWYVALKRTG | (SEQ ID NO.7) |
| 110 115 120 125 | |
| IL-1β NNKLEFESAQF..PNWYISTSQAE | (SEQ ID NO.8) |

Replacement of residues Arg118-Lys119-Tyr120-Thr121-Ser122 of FGF-2 with the human sequence Ala-Gln-Phe-Pro-Asn from the corresponding loop of the structural analogue IL-1β(115-119) was essentially performed as follows:

The plasmid DNA, pET-3a (M13), was subjected to BstB1 and Spl1 digestion, and the resulting larger DNA fragment was isolated using agarose gel electrophoresis. The DNA fragment was ligated, using T₄ DNA ligase, to a double-stranded DNA obtained by annealing two synthetic oligonucleotides: 5″-CGAACGATTG GAATCTAATA ACTACAATAC GTACCGGTCT GCGCAGTTTC CTAACTGGTA TGTGGCACTT AAGC-3′ (SEQ ID NO. 9) and 5′ GTACGCTTAA GTGCCACATA CCAGTTAGGA AACTGCGCAG ACCGGTACGT ATTGTAGTTA TTAGATTCCA ATCGTT-3′ (SEQ ID NO. 10), that contain termini compatible with those generated by BstB1 and Spl1 digestion. The ligation product was used to transfer Escherichia coli (strain DH5α) cells. The desired mutant plasmid (FGF-2LI) was selected for on the basis of susceptibility to cleavage at the newly introduced Afl2 restriction site (underlined above).

FGF-2LI with and without signal peptide were constructed by using a polymerase chain reaction (PCR)-based method. In order to add the FGF-4 signal peptide sequences to the 5′ of FGF-2LI and to ensure that the signal peptide will be cleaved from FGF-2LI protein, the gene cassette used by Forough R. et al for getting the secreted FGF-1 was employed. Using a primer (pF1B: 5′-CGGGATCCGC CCATGGCGGG GCCCGGGACG GC-3′ (SEQ ID NO. 11) matching the 5′ portion of the FGF-4 signal peptide and a second primer (pF2R: 5′-CGGAATTCTG TGAAGGTGGT GATTTCCC-3′) (SEQ ID NO. 12) to the 5′ portion of FGF-1, we synthesized, by PCR, a DNA fragment containing a Bam HI site at the 5′ end of the FGF-4 signal peptide sequences followed by the first ten amino acids of FGF-1 and an EcoRI site at the 3′ end. Using another pair of primers (pF3R: 5′-CGGAATTCAT GGCTGAAGGG GAAATCACC-3′ (SEQ ID NO. 13) and pF4HA: 5′-GCTCTAGATT AGGCGTAGTC TGGGACGTCG TATGGGTAGC TCTTAGCAGA CATTGGAAGA AAAAG-3′ (SEQ ID NO. 14)) matching the sequences of 5′- and 3′- of FGF-2LI, respectively, we obtained a second DNA fragment which has an EcoRI site at the 5′ end and an influenzae hemoagglutinin (HA) tag plus an XbaI site at the 3′ end of the FGF-2LI. These two fragments were then subcloned into pcDNA3 vector at a BamHI and XbaI site by three molecule ligation. The plasmid pFGF-2LI/cDNA3 which was similar to pSPFGF-2LI/cDNA3 except that it has no signal peptide was subcloned in a similar manner. Both plasmids were then sequenced to confirm the correction of the inserts. Both FGF-2LI fragments were then released from pcDNA3 by digestion with BamHI and XbaI and subcloned into pACCMVpLpASR(+) (pACSR for simplicity) which is a shuttle vector for making recombinant virus. Recombinant virus-and injectable vector were prepared essentially as described in Example 2. Gene transfer was performed as described in Example 5 (using 8 animals for FGF-2LI sp+and 6 animals for FGF-2LI sp−, with the lacZ vector serving as a control, all with 10¹¹ to 10¹² viral particles).

Results Using FGF-2 Muteins

Two weeks after gene transfer with FGF-2LI+sp, the peak contrast ratio (LCx/LV) during pacing stress at 200 bpm was significantly improved compared to pre-gene transfer. FIG. 13 shows results using intracoronary gene transfer of recombinant adenovirus expressing lacZ, FGF-5, FGF-2LI+sp, FGF-2LI −sp, and FGF-4 for comparison. The black bar on the right side in FIG. 13 shows the normal flow ratio using this method. FGF-2LI+sp normalized peak contrast flow ratio in these animals.

Percent wall thickening was also improved two weeks after intracoronary delivery of a recombinant adenovirus expressing FGF-2LI+sp. FIG. 11 shows results using intracoronary gene transfer of recombinant adenovirus expressing lacZ, FGF-5, FGF-2LI+sp, FGF-2LI−sp, and FGF-4 for comparison. The black bar on the right side in FIG. 11 shows the normal percent wall thickening before pacing-induced stress. FGF-2LI+sp improved regional function to a degree that was statistically indistinguishable from FGF-5. Although there was some improvement noted after gene transfer with FGF-2LI−sp, the improvement with the signal peptide containing transgene was superior (FIG. 13).

1. A method for increasing contractile function in the heart of a patient, comprising delivering a transgene encoding an angiogenic protein or peptide to the myocardium of the patient by introducing a vector comprising the transgene into at least one coronary artery, wherein the transgene is delivered to the myocardium and expressed, and contractile function in the heart is increased.

2. The method of claim 1, wherein the vector is introduced from a catheter conducted into the lumen of one or more coronary arteries.

3. The method of claim 2, wherein the vector is injected from the tip of said catheter.

4. The method of claim 1, wherein the introduction of vector comprises injecting the vector into the lumen of at least two coronary arteries supplying blood to the myocardium.

5. The method of claim 4, wherein the vector is introduced into at least one right coronary artery and at least one left coronary artery.

6. The method of claim 3, wherein the vector is introduced by injection from a catheter conducted at least about 1 cm into the lumen of said arteries.

7. The method of claim 6, wherein the vector is introduced into at least one right coronary artery and at least one left coronary artery.

8-56. (canceled)

57. A method for increasing blood flow in an ischemic tissue of a patient, comprising delivering a transgene encoding an angiogenic protein or peptide to an ischemic region of said tissue by introducing a vector comprising the transgene to said tissue, whereby the transgene is expressed in the tissue, and blood flow in the tissue is increased.

58. The method of claim 57, wherein the vector is introduced into a tissue by anterograde perfusion from a catheter placed into a conduit delivering blood to the tissue.

59. The method of claim 57, wherein the vector is introduced into a tissue by retrograde perfusion from a catheter placed into a conduit receiving blood from the tissue.

60. The method of claim 57, wherein the ischemic tissue comprises muscle cells and wherein increasing blood flow within the ischemic tissue results in increased contractile function.

61. The method of claim 60, wherein the muscle cells are cardiac myocytes.

62. The method of claim 62, wherein the blood vessel is selected from the group consisting of a coronary artery and a femoral artery.

63. The method of claim 57, wherein the vector is introduced by injecting a solution comprising the vector into skeletal muscle, wherein the angiogenic protein or peptide causes an increase in blood flow and a decrease in ischemia in the tissue.

64-120. (canceled)

121. A gene therapy composition comprising a vector containing a transgene encoding an angiogenic protein or peptide.

122. The composition of claim 121, wherein said vector is a viral vector.

123. The composition of claim 122, wherein said vector is a replication-deficient viral vector.

124. The composition of claim 122, wherein said vector is an adenovirus vector.

125. The composition of claim 124, wherein said vector is a replication-deficient adenovirus vector.

126. The composition of claim 124, comprising about 10⁷ to about 10¹³ adenovirus vector particles.

127-156. (canceled)
